(19) United States
(12) Patent Application Publication
(10) Pub. No.: US 2010/0242139 A1
(43) Pub. Date: Sep. 23, 2010

(54) XENOHORMESIS BASED COMPOSITIONS AND METHODS

(75) Inventors: David A. Sinclair, Chestnut Hill, MA (US); Joseph A. Baur, Merion Station, PA (US); Sean M. Armour, Roxbury Crossing, MA (US)

Correspondence Address: WOLF GREENFIELD & SACKS, P.C., 600 ATLANTIC AVENUE, BOSTON, MA, 02210-2206, US

(73) Assignee: President and Fellows of Harvard College, Cambridge, MA (US)

(21) Appl. No.: 12/087,641

(22) PCT Filed: Jan. 12, 2007
(86) PCT No.: PCT/US2007/060513

§ 371 Date: Mar. 12, 2009

Related U.S. Application Data
(60) Provisional application No. 60/758,703, filed on Jan. 13, 2006.

(51) Int. Cl. A01H 5/00; C12Q 1/02; C12Q 1/48; G01N 33/68

(52) U.S. Cl. 800/295; 435/29; 435/15; 436/86

(57) ABSTRACT

The present invention provides methods and compositions for improving the health of subjects, such as their resistance to stress.

### RELATED APPLICATIONS

This application claims the benefit of priority to U.S. provisional application 60/758,703 filed Jan. 13, 2006, which is herein incorporated by reference in its entirety.

### GOVERNMENT SUPPORT

This invention was made with government support under Grant number R01 AG19972 awarded by the National Institutes of Health. The government has certain rights in this invention.

### BACKGROUND

There is now good evidence from model organisms that the pace of aging can be regulated (Kenyon, C. Cell 105, 165-168 (2001)). Longevity regulatory genes have been identified in many eukaryotes, including rodents, flies, nematode worms and even single-celled organisms such as baker's yeast (reviewed in Sinclair, D. A. Mech Ageing Dev 123, 857-67 (2002); Hekimi, S. & Guarente Science 299, 1351-4 (2003)). These genes appear to be part of an evolutionarily conserved longevity pathway that evolved to promote survival in response to deteriorating environmental conditions or stress (Kenyon, C. Cell 105, 165-168 (2001); Guarente, L. & Kenyon, C. Nature 408, 255-62. (2000)). Stress may include starvation, irradiation, heat or toxin exposure. In these organisms, lifespan extension is dependent on Sir2, a conserved deacetylase proposed to underlie the beneficial effects of caloric restriction. Resveratrol (3,5,4′-trihydroxystilbene), a constituent of red wine, has been identified to activate sirtuin deacetylase and to extend the life spans of lower organisms, however, the mechanism by which resveratrol exerts its range of beneficial effects is not yet clear.

Understanding how organisms deal with stress and enter a state of stress-resistance is an area of active investigation. There remains an unmet need to identify molecules that induce stress resistance and promote longevity as well as the cellular targets that may be modulated in response to stress.

### SUMMARY

Provided herein are methods and compositions for improving the health of subjects, such as their resistance to stress. Methods for determining their health condition are also provided herein, as well as methods for identifying additional health beneficial compositions.

### BRIEF DESCRIPTION OF THE FIGURES

FIG. 1 shows graphs of the activity (as a percentage of the control) of human JAK2 in the presence of 10 and 100 μM ATP (panels A and B, respectively) and various concentrations of resveratrol.

FIG. 2 shows graphs of the activity (as a percentage of the control) of human pim 1 in the presence of 10 or 100 μM ATP (panels A and B, respectively) and various concentrations of resveratrol.

FIG. 3 shows graphs of the activity (as a percentage of the control) of human pim 2 (panel A) and a control enzyme (Aurora A) (panel B) in the presence of 10 μM ATP and various concentrations of 4-glucoronide, a metabolite of resveratrol.

FIG. 4 shows graphs of the activity (as a percentage of the control) of human p70S6K in the presence of 10 or 100 μM ATP (panels A and B, respectively) and various concentrations of resveratrol.

FIG. 5 shows graphs of the activity (as a percentage of the control) of human JAK3 (panel A) and NLK (panel B) in the presence of 10 μM ATP and various concentrations of resveratrol.

FIG. 6 is a graph showing dose dependent inhibition of the growth of hematopoietic cell line FL5.12 by resveratrol at 0.1, 1, 10, and 100 μM.

FIGS. 7(a-h) depicts the name or structures of exemplary xenohormetic compounds.

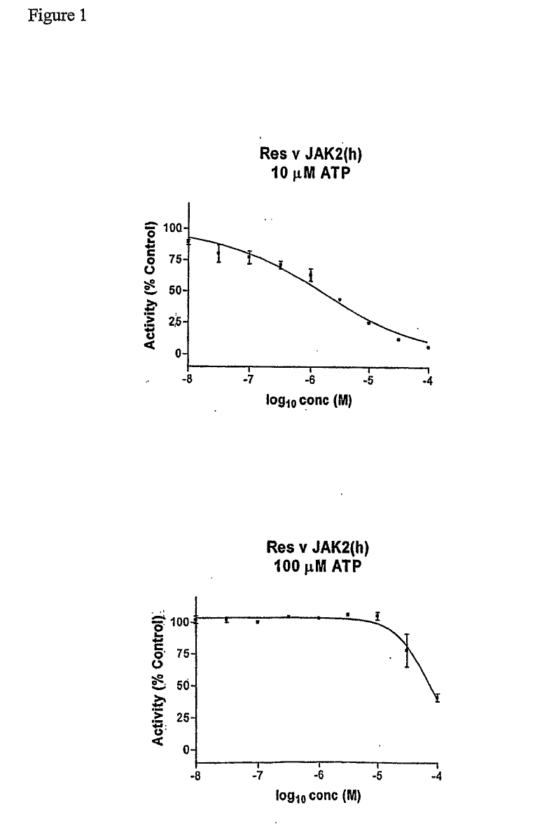

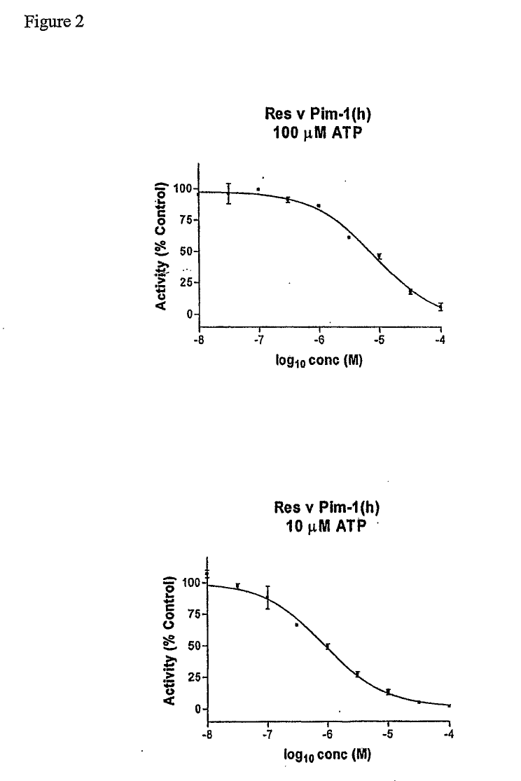

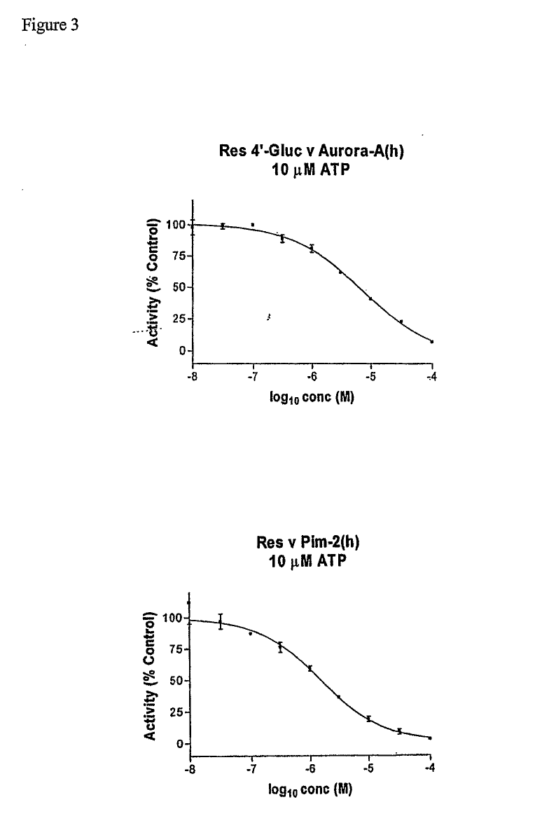

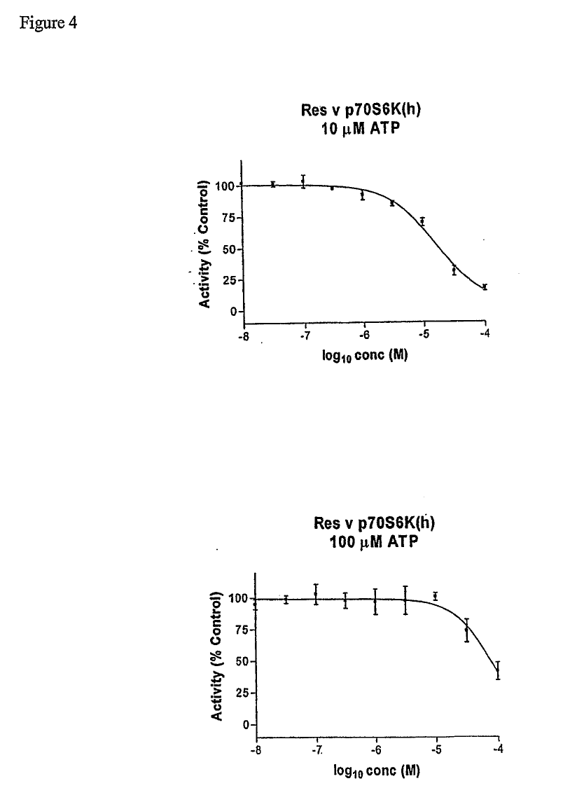

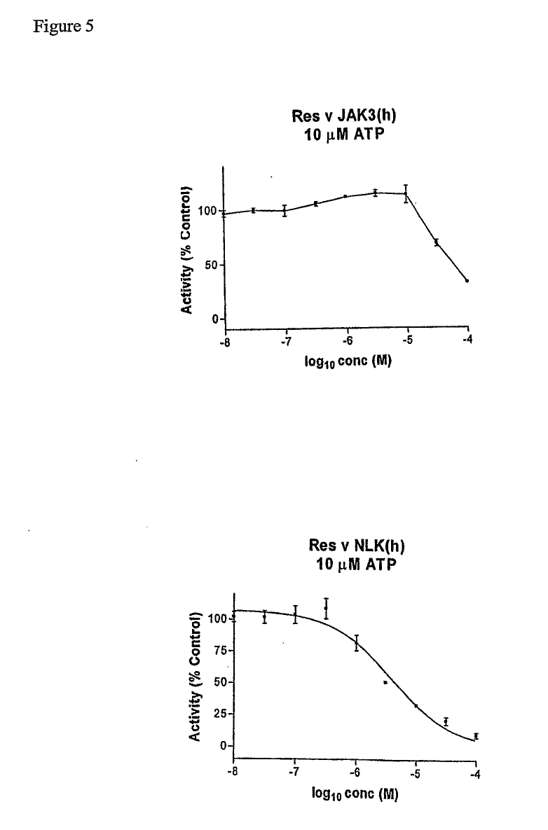

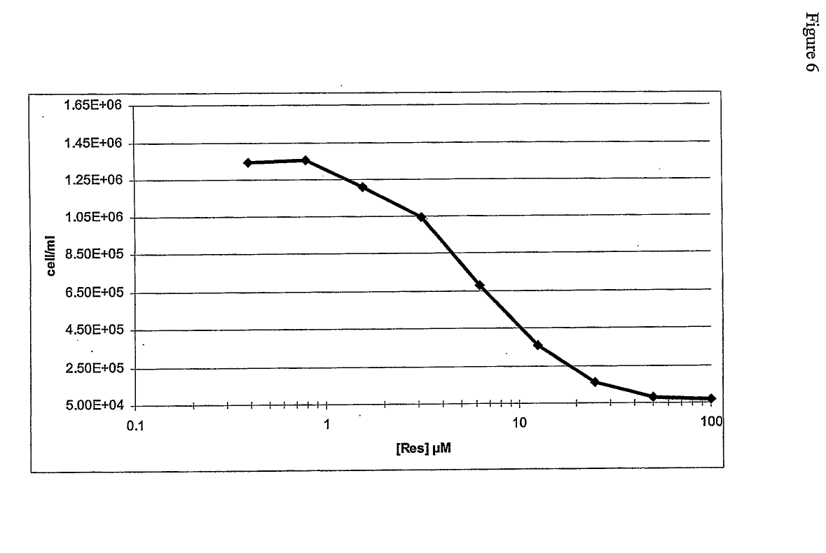

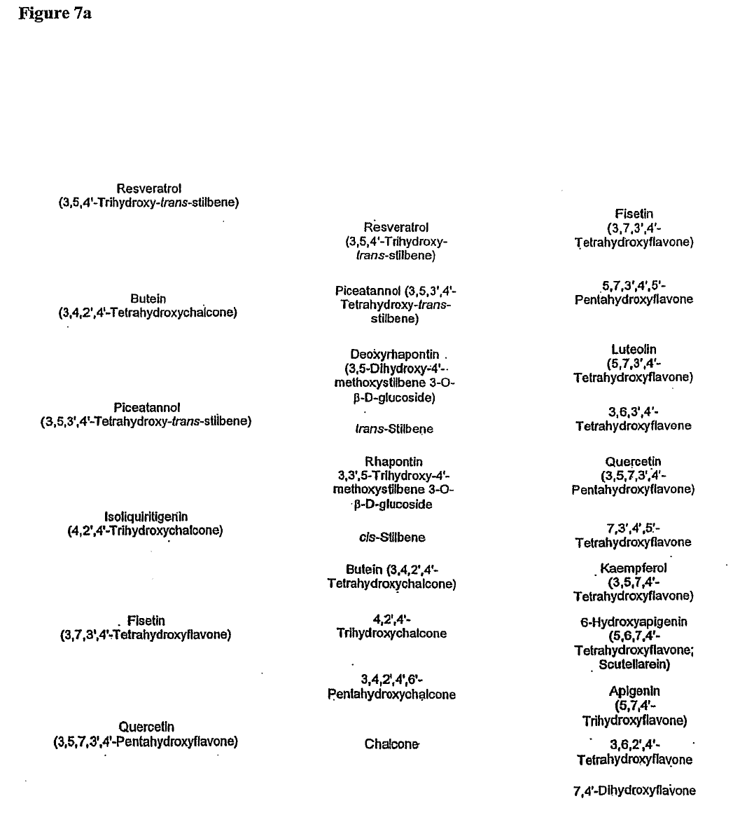

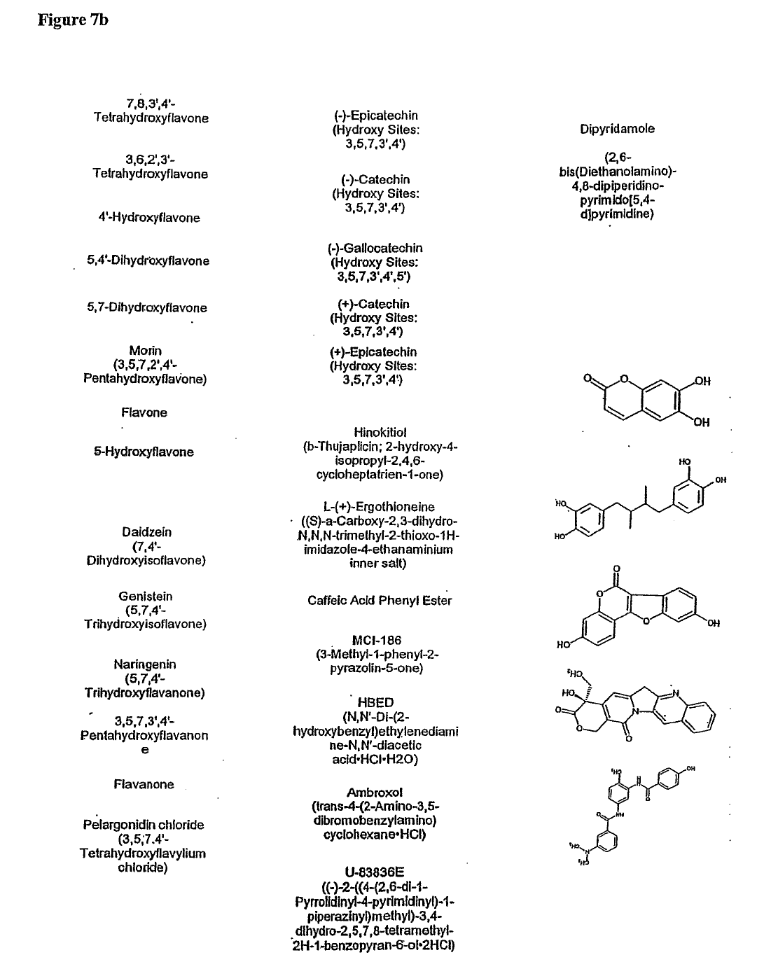

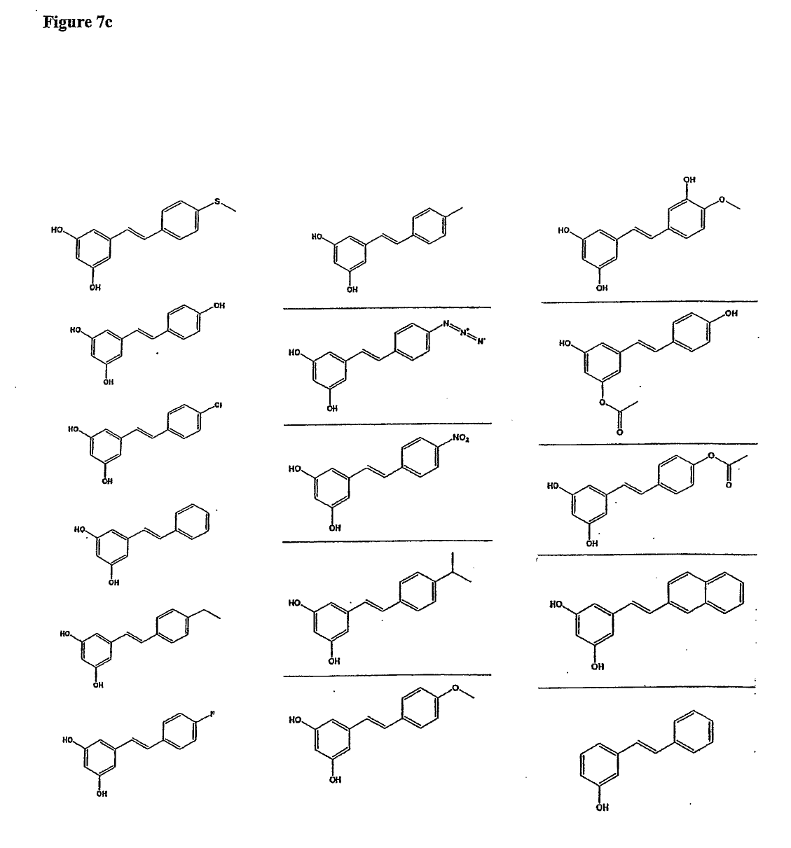

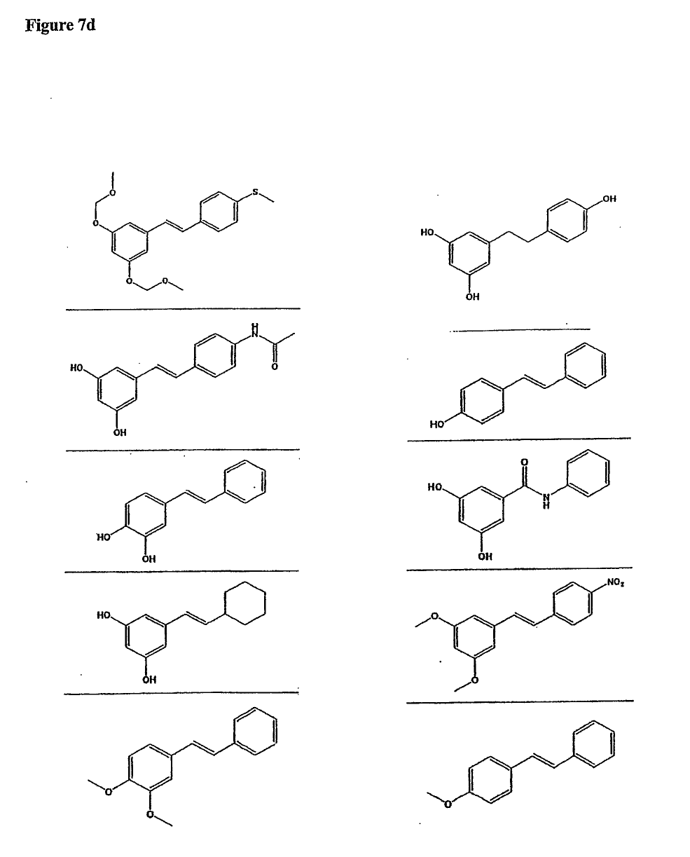

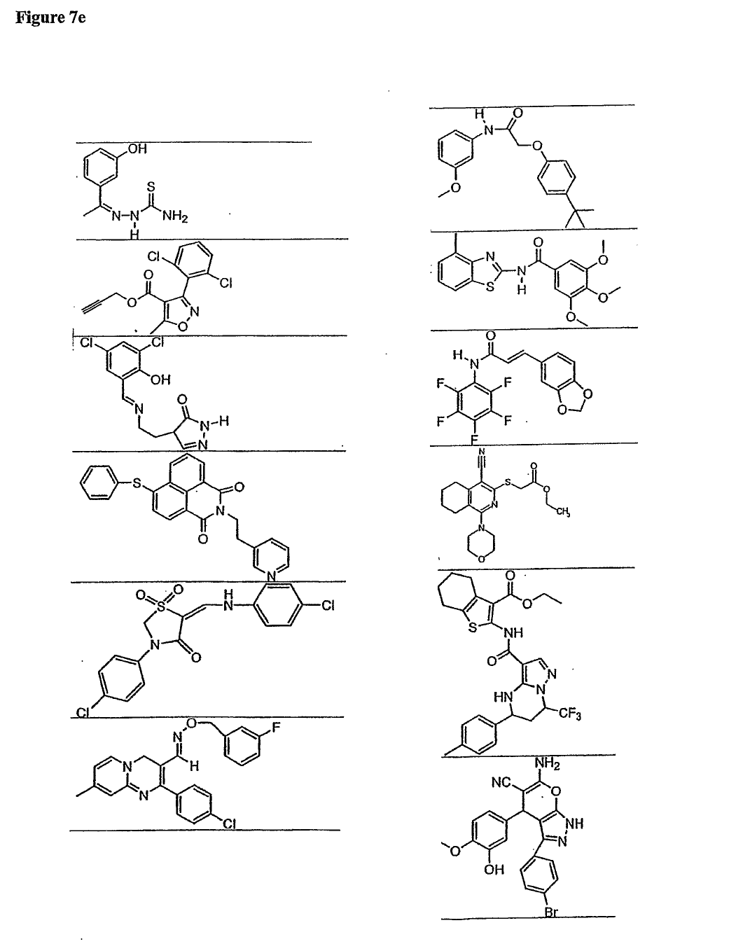

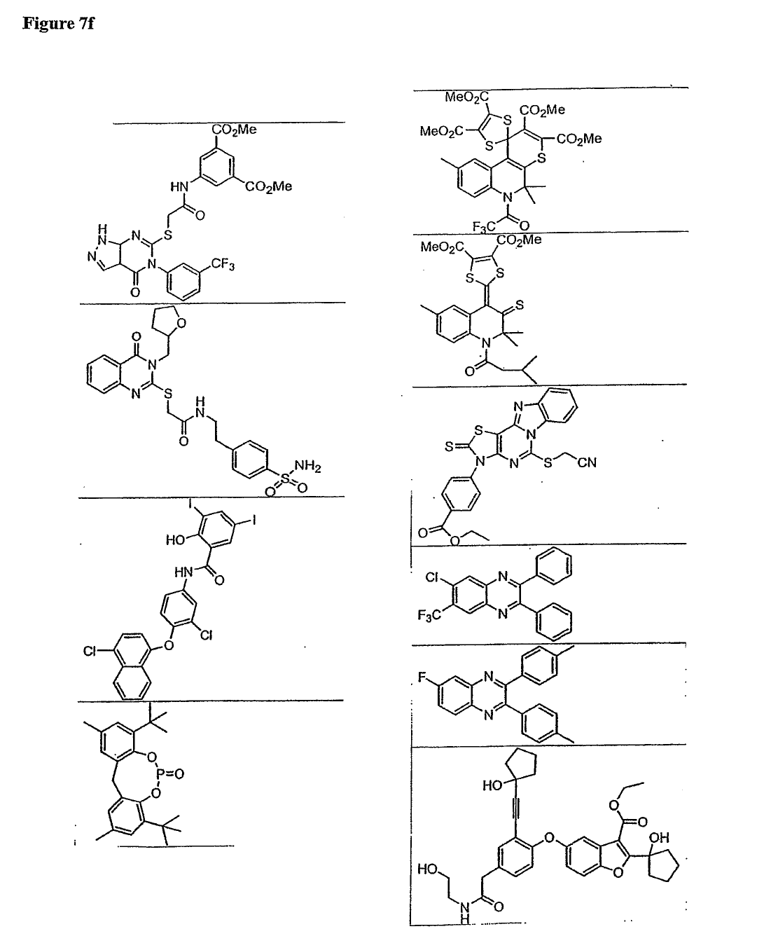

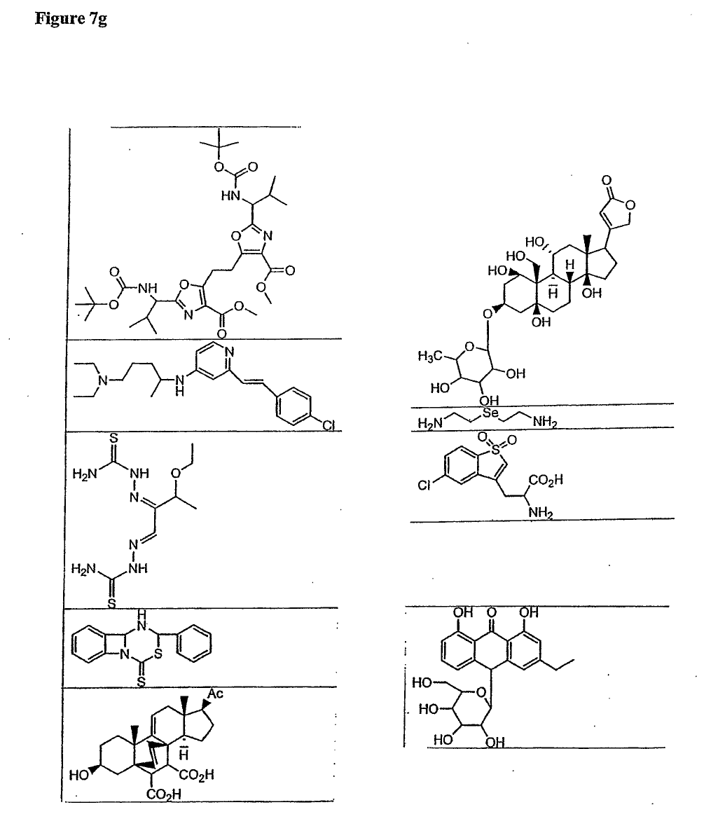

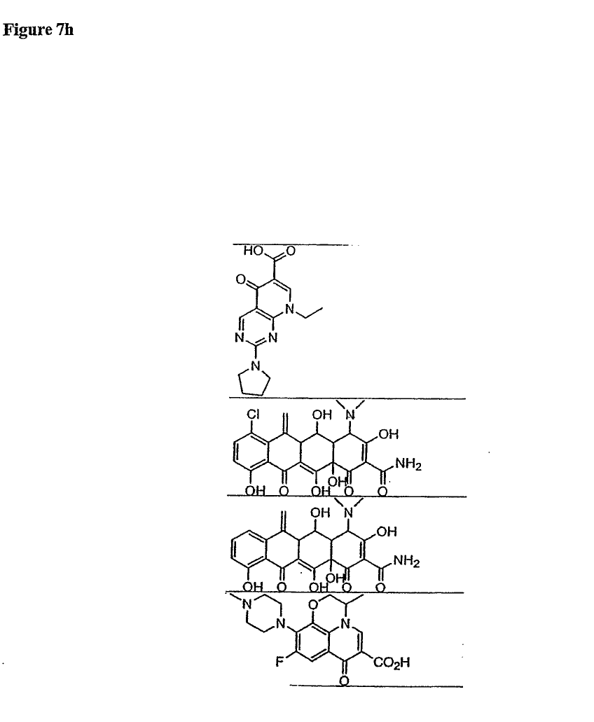

### DETAILED DESCRIPTION OF THE INVENTION

Studies on caloric restriction have thoroughly established that organisms, including mammals, are capable of entering a state of increased stress-resistance that improves health and survival when energy intake is low (1,2). From an evolutionary standpoint, it has been suggested that this might represent an adaptive response to changing environmental conditions. A central concept in many theories of aging is the idea that evolution selects only for the ability to reproduce, and consequently traits that are deleterious late in life will never be eliminated. However, the exception to this rule may be cases where resources become severely limiting, such that the energy for reproduction is not readily available and offspring are less likely to survive. Under such circumstances, the best overall strategy for reproduction may be to focus on the protection and maintenance of somatic tissues, effectively slowing the aging process, until a more favorable environment for reproduction exists (3).

Xenohormesis is a hypothesis which essentially states that organisms have evolved to respond to chemical cues in their diet to gain advance warning of a deteriorating environment (4). Phytoalexins such as resveratrol are produced in plants in response to various stresses including injury, infection, and UV light (5-8). Since these molecules increase when the food source is threatened, it is likely that animals eating these plants experience an increased exposure to these molecules before famine or other stresses, and that responding to their presence in the diet, rather than waiting for a direct stress, might confer an evolutionary advantage.

According to xenohormesis, stressed plants will form an abundant reservoir for medicinal compounds that trigger part or all of the caloric restriction response. Indeed, the beneficial effects of a variety of phytochemicals, many of them structurally related to resveratrol, are currently gaining the attention of the scientific community. Although none have yet been studied in as much detail, molecules such as catchetin, quercetin, and pterostilbene are beneficial alone and may have additive or even synergistic effects in combination with resveratrol.

Since xenohormesis predicts that animals have adapted to respond in a particular way to the presence of phytochemicals from stressed plants, rather than having random effects occur with each phytochemical, we can predict that a variety of different molecules (even when structurally unrelated) should produce similar effects. By screening a large number of these molecules against panels of enzyme activities or gene expression patterns, we should be able to determine a consensus pattern that corresponds to the healthier state induced by phytochemicals from stressed plants. Our invention would involve the identification of this pattern and its use to evaluate potential xenohormetic molecules, and design new drugs that might tap into pathways related to xenohormesis.

By having a known target profile, it should be possible to identify xenohormetic molecules and develop an idealized formulation for obtaining the benefits of such molecules. This could involve identifying a single molecule or a mixture that most closely approximates the consensus profile.

By discovering what these molecules hit in the proteome, it may be possible to design new, more potent drugs, foodstuffs, neutraceuticals or cosmeceuticals (or cosmecuticals) that hit the same targets.

By comparing side effects of new drugs to our xenohormetic effects, it may be possible to predict in advance whether they will be harmful, neutral, or even beneficial. Our data will be useful for this even if the consensus profile is not entirely clear since many of the individual molecules we would test, including resveratrol, are known to be non-toxic.

Our invention predicts that stressed plants will harbor a bounty of xenohormetic molecules that can be isolated for example using HPLC, FPLC, and other fractionization methods and identified by comparison to pre-defined xenohormetic profiles of molecular interactions and modulations of protein activity.

### DEFINITIONS

The term “cis” is art-recognized and refers to the arrangement of two atoms or groups around a double bond such that the atoms or groups are on the same side of the double bond. Cis configurations are often labeled as (Z) configurations.

The term “trans” is art-recognized and refers to the arrangement of two atoms or groups around a double bond such that the atoms or groups are on the opposite sides of a double bond. Trans configurations are often labeled as (E) configurations.

The term “covalent bond” is art-recognized and refers to a bond between two atoms where electrons are attracted electrostatically to both nuclei of the two atoms, and the net effect of increased electron density between the nuclei counterbalances the internuclear repulsion. The term covalent bond includes coordinate bonds when the bond is with a metal ion.

The term “therapeutic agent” is art-recognized and refers to any chemical moiety that is a biologically, physiologically, or pharmacologically active substance that acts locally or systemically in a subject. The term thus means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a at a reasonable benefit/risk ratio applicable to such treatment.

The term “synthetic” is art-recognized and refers to production by in vitro chemical or enzymatic synthesis.

The term “meso compound” is art-recognized and refers to a chemical compound which has at least two chiral centers but is achiral due to a plane or point of symmetry.

The term “chiral” is art-recognized and refers to molecules which have the property of non-superimposability of the mirror image partner, while the term “achiral” refers to molecules which are superimposable on their mirror image partner. A “prochiral molecule” is a molecule which has the potential to be converted to a chiral molecule in a particular process.

The term “stereoisomers” is art-recognized and refers to compounds which have identical chemical constitution, but differ with regard to the arrangement of the atoms or groups in space. In particular, “enantiomers” refer to two stereoisomers of a compound which are non-superimposable mirror images of one another. “Diastereomers”, on the other hand, refers to stereoisomers with two or more centers of dissymmetry and whose molecules are not mirror images of one another.

Furthermore, a “stereoselective process” is one which produces a particular stereoisomer of a reaction product in preference to other possible stereoisomers of that product. An “enantioselective process” is one which favors production of one of the two possible enantiomers of a reaction product.

The term “regioisomers” is art-recognized and refers to compounds which have the same molecular formula but differ in the connectivity of the atoms. Accordingly, a “regioselective process” is one which favors the production of a particular regioisomer over others, e.g., the reaction produces a statistically significant increase in the yield of a certain regioisomer.

The term “epimers” is art-recognized and refers to molecules with identical chemical constitution and containing more than one stereocenter, but which differ in configuration at only one of these stereocenters.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “structure-activity relationship” or “(SAR)” is art-recognized and refers to the way in which altering the molecular structure of a drug or other compound alters its biological activity, e.g., its interaction with a receptor, enzyme, nucleic acid or other target and the like.

The term “aliphatic” is art-recognized and refers to a linear, branched, cyclic alkane, alkene, or alkyne. In certain embodiments, aliphatic groups in the present compounds are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” is also defined to include halosubstituted alkyls.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “heteroatom” is art-recognized and refers to an atom of any element other than carbon or hydrogen. Illustrative heteroatoms include boron, nitrogen, oxygen, phosphorus, sulfur and selenium.

The term “aryl” is art-recognized and refers to 5-, 6- and 7-membered single-ring aromatic groups that may include from zero to four heteroatoms, for example, benzene, naphtalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carbolise, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloallcyl, hydroxyl, amino, nitro, sulthydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas:

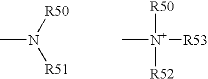

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula:

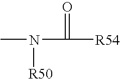

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

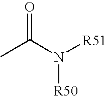

wherein R50 and R51 are as defined above. Certain embodiments of amides may not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas:

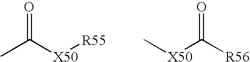

wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula:

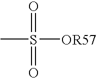

in which R57 is an electron pair, hydrogen, alkyl, cycloalkyl, or aryl.

The term “sulfate” is art recognized and includes a moiety that may be represented by the general formula:

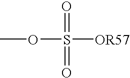

in which R57 is as defined above.

The term “sulfonamido” is art recognized and includes a moiety that may be represented by the general formula:

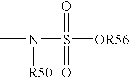

in which R50 and R56 are as defined above.

The term “sulfamoyl” is art-recognized and refers to a moiety that may be represented by the general formula:

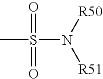

in which R50 and R51 are as defined above.

The term “sulfonyl” is art-recognized and refers to a moiety that may be represented by the general formula:

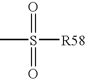

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

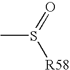

in which R58 is defined above.

The term “phosphoryl” is art-recognized and may in general be represented by the formula:

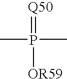

wherein Q50 represents S or O, and R59 represents hydrogen, a lower alkyl or an aryl. When used to substitute, e.g., an alkyl, the phosphoryl group of the phosphorylalkyl may be represented by the general formulas:

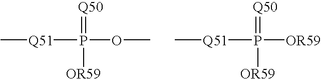

wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas:

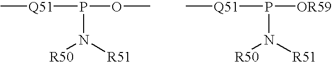

wherein Q51, R50, R51 and R59 are as defined above.

The term “phosphonamidite” is art-recognized and may be represented in the general formulas:

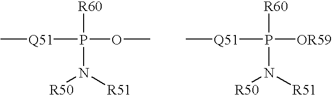

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions described herein may exist in particular geometric or stereoisomeric forms. In addition, compounds may also be optically active. Contemplated herein are all such compounds, including cis- and trans-isomers, R- and S-enantiomers, diastereomers, (D)-isomers, (L)-isomers, the racemic mixtures thereof, and other mixtures thereof. Additional asymmetric carbon atoms may be present in a substituent such as an alkyl group. All such isomers, as well as mixtures thereof, are encompassed herein.

If, for instance, a particular enantiomer of a compound is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. Heteroatoms such as nitrogen may have hydrogen substituents and/or any permissible substituents of organic compounds described herein which satisfy the valences of the heteroatoms. Compounds are not intended to be limited in any manner by the permissible substituents of organic compounds.

The chemical elements are identified in accordance with the Periodic Table of the Elements, CAS version, Handbook of Chemistry and Physics, 67th Ed., 1986-87, inside cover.

The term “protecting group” is art-recognized and refers to temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed by Greene and Wuts in Protective Groups in Organic Synthesis (2^{nd} ed., Wiley: New York, 1991).

The term “hydroxyl-protecting group” is art-recognized and refers to those groups intended to protect a hydroxyl group against undesirable reactions during synthetic procedures and includes, for example, benzyl or other suitable esters or ethers groups known in the art.

The term “carboxyl-protecting group” is art-recognized and refers to those groups intended to protect a carboxylic acid group, such as the C-terminus of an amino acid or peptide or an acidic or hydroxyl azepine ring substituent, against undesirable reactions during synthetic procedures and includes. Examples for protecting groups for carboxyl groups involve, for example, benzyl ester, cyclohexyl ester, 4-nitrobenzyl ester, t-butyl ester, 4-pyridylmethyl ester, and the like.

The term “amino-blocking group” is art-recognized and refers to a group which will prevent an amino group from participating in a reaction carried out on some other functional group, but which can be removed from the amine when desired. Such groups are discussed by in Ch. 7 of Greene and Wuts, cited above, and by Barton, Protective Groups in Organic Chemistry ch. 2 (McOmie, ed., Plenum Press, New York, 1973). Examples of suitable groups include acyl protecting groups such as, to illustrate, formyl, dansyl, acetyl, benzoyl, trifluoroacetyl, succinyl, methoxysuccinyl, benzyl and substituted benzyl such as 3,4-dimethoxybenzyl, o-nitrobenzyl, and triphenylmethyl; those of the formula —COOR where R includes such groups as methyl, ethyl, propyl, isopropyl, 2,2,2-trichloroethyl, 1-methyl-1-phenylethyl, isobutyl, t-butyl, t-amyl, vinyl, allyl, phenyl, benzyl, p-nitrobenzyl, o-nitrobenzyl, and 2,4-dichlorobenzyl; acyl groups and substituted acyl such as formyl, acetyl, chloroacetyl, dichloroacetyl, trichloroacetyl, trifluoroacetyl, benzoyl, and p-methoxybenzoyl; and other groups such as methanesulfonyl, p-toluenesulfonyl, p-bromobenzenesulfonyl, p-nitrophenylethyl, and p-toluenesulfonyl-aminocarbonyl. Preferred amino-blocking groups are benzyl (—CH₂C₆H₅), acyl [C(O)R1] or SiR1₃ where R1 is C₁-C₄ alkyl, halomethyl, or 2-halo-substituted-(C₂-C₄ alkoxy), aromatic urethane protecting groups as, for example, carbonylbenzyloxy (Cbz); and aliphatic urethane protecting groups such as t-butyloxycarbonyl (Boc) or 9-fluorenylmethoxycarbonyl (FMOC).

The definition of each expression, e.g. lower alkyl, m, n, p and the like, when it occurs more than once in any structure, is intended to be independent of its definition elsewhere in the same structure.

The term “electron-withdrawing group” is art-recognized, and refers to the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59 (McGraw Hill Book Company: New York, 1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “pharmaceutically-acceptable salts” is art-recognized and refers to the relatively non-toxic, inorganic and organic acid addition salts of compounds, including, for example, those contained in compositions described herein.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

### Exemplary Xenohormetic Molecules and Targets

Xenohormetic molecules include molecules that are produced in plants or products thereof, such as fruit, vegetables, flowers, and grains, in response to a stress condition. Stress may be any non-optimal condition for growth, development or reproduction, or a non physiological condition, e.g., heat, dehydration, infection, starvation, irradiation, injury, excessive light, and cold, e.g., below freezing temperatures. A “stress condition” can also be exposure to heatshock; osmotic stress; a DNA damaging agent; inadequate salt level; inadequate nitrogen levels; inadequate nutrient level; radiation or a toxic compound, e.g., a toxin or chemical warfare agent (such as dirty bombs and other weapons that may be used in bioterrorism). “Inadequate levels” refer to levels that result in non-optimal condition for growth, development or reproduction.

Xenohormetic molecules modulate the activity of xenohormetic targets. For example, a xenohormetic molecule may inhibit the activity of a protein kinase, e.g., JAK2, Pim1, Pim 2, S6K, NLK, and Rsk2, (see Examples). A xenohormetic molecule may also stimulate the activity of other xenohormetic targets, such as a sirtuin, e.g., SIRT1 in humans.

JAK2 (Janus kinase 2; GeneID: 3717) is a protein tyrosine kinase that is involved in a specific subset of cytokine receptor signaling pathways. The nucleotide and amino acid sequences of human JAK2 are set forth in GenBank Accession numbers NM_{—}004972 and NP_{—}004963, respectively.

Pim-1 (GeneID: 5292), also referred to as oncogene PIM1; pim-1 kinase 44 kDa isoform; and pim-1 oncogene (proviral integration site 1), encodes a protein kinase that is upregulated in tumors of the lymphatic system and prostate cancer. The nucleotide and amino acid sequences of human Pim 1 are set forth in GenBank Accession numbers NM_{—}002648 and NP_{—}002639, respectively.

Pim-2 (GeneID: 11040) is also referred to as PIM2 oncogene. The nucleotide and amino acid sequences of human Pim 2 are set forth in GenBank Accession numbers NM_{—}006875 and NP_{—}006866, respectively.

S6K (GeneID: 6198) is also referred to as RPS6 KB1 ribosomal protein S6 kinase 70 kDa polypeptide 1; PS6K; S6K1; STK14A; p70-S6K; p70-alpha; and p70(S6K)-alpha, is a member of the RSK (ribosomal S6 kinase) family of serine/threonine kinases. This kinase contains 2 non-identical kinase catalytic domains and phosphorylates several residues of the S6 ribosomal protein. The kinase activity of this protein leads to an increase in protein synthesis and cell proliferation. The nucleotide and amino acid sequences of human S6K are set forth in GenBank Accession numbers NM_{—}003161 and NP_{—}003152, respectively.

NLK (GeneID: 51701) is also referred to as nemo like kinase. The nucleotide and amino acid sequences of human NLK are set forth in GenBank Accession numbers NM_{—}016231 and NP_{—}057315, respectively.

Rsk2 (GeneID: 6197) is also referred to as RP11-393H10.3, HU-3, ISPK-1, MAPKAPK1B, MRX19, RSK, RSK2, S6K-alpha3, p90-RSK3, pp 90RSK2, insulin-stimulated protein kinase 1, and ribosomal protein S6 kinase, 9010, polypeptide 3. The nucleotide and amino acid sequences of human Rsk2 are set forth in GenBank Accession numbers NM_{—}004586 and NP_{—}004577, respectively.

Another xenohormetic target is AMP kinase, also referred to as AMP-activated protein kinase or AMPK, since it has been shown to be activated by resveratrol, and this activation is independent of Sirtl (see e.g., Baur, J. A., et al., Nature, 444:337-42 (2006)).

Yet another xenohormetic target is IRS1 (GeneID: 3667), also referred to as insulin receptor substrate 1 and MRS-1. Expression of IRS1 has been shown to be inhibited by resveratrol. The nucleotide and amino acid sequences of human IRS1 are set forth in GenBank Accession numbers NM_{—}005544 and NP_{—}005535, respectively.

Other xenohormetic targets include the following targets which are inhibited by resveratrol: Quinone Reductase 2, also referred to as QR2; aryl hydrocarbon receptor, also referred to as AhR; CYP3A4; CYP1B1; CYP2C19; CYP1A1; CYP1A2; Ribonucleotide Reductase; c-Src; PKC; Protein Kinase Cδ; and Cyclooxygenase (COX-1 and COX-2).

Still other xenohormetic targets include the following targets which are activated by resveratrol: QR1, GST, catalase, GSH, and SOD, paraoxonase-1; Estrogen Receptor; and SIRT1.

Xenohormetic targets may also include transcription factors. Transcription factors, whose expression has been shown to be altered following exposure to resveratrol include, but are not limited to, ELK1, a member of ETS oncogene family, Sp1, NRF1 (Nuclear respiratory factor 1), E4F1 (E4F transcription factor 1), GABP (GA binding protein transcription factor), FREAC2:FOXF2—Forkhead box F2, NFY (Nuclear transcription factor Y), ERR1, YY1, MYC:V-myc myelocytomatosis viral oncogene homolog (avian), PAX3 (Paired box gene 3/Waardenburg syndrome 1), LEF1 (Lymphoid enhancer-binding factor 1), FOXO4:MLLT7—Myeloid/lymphoid or mixed-lineage leukemia (trithorax homolog, Drosophila) translocated to 7; HSF1 (Heat shock transcription factor 1), ETS2:V-ets erythroblastosis virus E26 oncogene homolog 2 (avian), NF1 (Neurofibromin 1, neurofibromatosis, von Recklinghausen disease, Watson disease), and MEIS1 (Meisl, myeloid ecotropic viral integration site 1 homolog (mouse)).

Further xenohormetic targets may also include transcription factors that bind to one of the consensus sites provided in Table 1. The consensus sites provided in Table 1 are short stretches of DNA isolated from the promoter regions of genes that are down-regulated or upregulated following exposure to resveratrol.

TABLE 1
Consensus sites for transcription
factor binding

| | Transcription Factor Binding |
|---|---|
| Consensus Site | to Consensus Site |
| SCGGAAGY | ELK1: ELK1, member of ETS |
| | oncogene family |
| GGGCGGR | SP1: Sp1 transcription factor |
| RCGCANGCGY | NRF1: Nuclear respiratory |
| | factor 1 |
| TGCGCANK | Unknown motif |
| ACTAYRNNNCCCR | Unknown motif |
| GTGACGY | E4F1: E4F transcription |
| | factor 1 |
| GKCGCNNNNNNNTGAYG | Unknown motif |
| MGGAAGTG | GABP: GA binding protein |
| | transcription factor |
| RTAAACA | FREAC2: FOXF2 - Forkhead box |
| | F2 |
| AAGWWRNYGGC | Unknown motif |
| GATTGGY | NFY: Nuclear transcription |
| | factor Y |
| TGACCTY | ERR1: ERR1 motif |
| GCCATNTTG | YY1: YY1 transcription factor |
| KMCATNNWGGA | Unknown motif |
| CACGTG | MYC: V-myc myelocytomatosis |
| | viral oncogene homolog (avian) |
| CGTSACG | PAX3: Paired box gene 3 |
| | (Waardenburg syndrome 1) |
| YGCGYRCGC | Unknown motif |
| CGGAARNGGCNG | Unknown motif |
| CCGNMNNTNACG | Unknown motif |
| YKACATTT | Unknown motif |
| YGTCCTTGR | Unknown motif |
| AACTTT | Unknown motif |
| CTTTGT | LEF1: Lymphoid enhancer- |
| | binding factor 1 |
| TTGTTT | FOXO4: MLLT7 - Myeloid/lymphoid |
| | or mixed-lineage leukemia |
| | (trithorax homolog, Drosophila); |
| | translocated to, 7 |
| GGCNKCCATNK | Unknown motif |
| RGAANNTTC | HSF1: Heat shock transcription |
| | factor 1 |
| GCGNNANTTCC | Unknown motif |
| MYAATNNNNNNNGGC | Unknown motif |
| YGCANTGCR | Unknown motif |
| AACYNNNNTTCCS | Unknown motif |
| CTGCAGY | Unknown motif |
| TNCATNTCCYR | Unknown motif |
| RYTTCCTG | ETS2: V-ets erythroblastosis |
| | virus E26 oncogene homolog |
| | 2 (avian) |
| TMTCGCGANR | Unknown motif |
| TGCCAAR | NF1: Neurofibromin 1 |
| | (neurofibromatosis, von |
| | Recklinghausen disease, |
| | Watson disease) |
| GGCNNMSMYNTTG | Unknown motif |
| CAGNYGKNAAA | Unknown motif |
| TGACAGNY | MEIS1: Meis1, myeloid ecotropic |
| | viral integration site 1 |
| | homolog (mouse) |
| GATGKMRGCG | Unknown motif |
| GGGNRMNNYCAT | Unknown motif |
| KCCGNSWTTT | Unknown motif |
| GGAANCGGAANY | Unknown motif |
| ATCMNTCCGY | Unknown motif |
| YATTNATC | Unknown motif |

Modulating, e.g., inhibiting, a protein kinase refers to modulating, e.g., inhibiting, the ability of the protein kinase to phosphorylate a target peptide or protein.

“Sirtuin deacetylase protein family members;” “Sir2 family members;” “Sir2 protein family members;” or “sirtuin proteins” includes yeast Sir2, Sir-2.1, and human SIRT1 and SIRT2 proteins. GenBank Accession numbers of the human sirtuins are set forth below. Other family members include the four additional yeast Sir2-like genes termed “HST genes” (homologues of Sir two) HST1, HST2, HST3 and HST4.

| name | | nucleotide sequence | amino acid sequence |
|---|---|---|---|
| SIRT1 | | NM_012238 | NP_036370 |
| SIRT2 | i1 | NM_012237 | NP_036369 |
| | i2 | NM_030593 | NP_085096 |
| SIRT3 | ia | NM_012239 | NP_036371 |
| | ib | NM_001017524 | NP_001017524 |
| SIRT4 | | NM_012240 | NP_036372 |
| SIRT5 | i1 | NM_012241 | NP_036373 |
| | i2 | NM_031244 | NP_112534 |
| SIRT6 | | NM_016539 | NP_057623 |
| SIRT7 | | NM_016538 | NP_057622 |

“Activating a sirtuin protein” refers to the action of producing an activated sirtuin protein, i.e., a sirtuin protein that is capable of performing at least one of its biological activities to at least some extent, e.g., with an increase of activity of at least about 10%, 50%, 2 fold or more. Biological activities of sirtuin proteins include deacetylation, e.g., of histones, PGC-1α and p53; extending lifespan of cells and organisms; increasing memory; mobilizing fat stores; lowering blood glucose levels; releasing insulin from pancreatic beta cells; providing neuroprotection; increasing genomic stability; silencing transcription; and controlling the segregation of oxidized proteins between mother and daughter cells.

Exemplary xenohormetic molecules include resveratrol, derivatives and analogs thereof, and any other molecule that increases the activity of a sirtuin, such as flavones, stilbenes, flavanones, isoflavanones, catechins, chalcones, tannins and anthocyanidins. Exemplary stilbenes include hydroxystilbenes, such as trihydroxystilbenes, e.g., 3,5,4′-trihydroxystilbene (“resveratrol”). Resveratrol is also known as 3,4′,5-stilbenetriol. Other molecules that activate a sirtuin include tetrahydroxystilbenes, e.g., piceatannol; hydroxychalones including trihydroxychalones, such as isoliquiritigenin, and tetrahydroxychalones, such as butein; hydroxyflavones including tetrahydroxyflavones, such as fisetin; and pentahydroxyflavones, such as quercetin. Additional compounds are those described, e.g., in U.S. published applications numbers 2005/0096256, 2005/0096256, and PCT applications publication numbers WO 05/002672 and WO 05/002555, all of which are specifically incorporated by reference herein.

Compounds for use as xenhormetic molecules include resveratrol (3,5,4′-trihydroxystilbene), its chemical derivatives, and structural neighbors as selective kinase inhibitors. A xenohormetic compound may be a compound represented by formula 1 having xenohormetic activity, e.g., providing resistance to disease or stress:

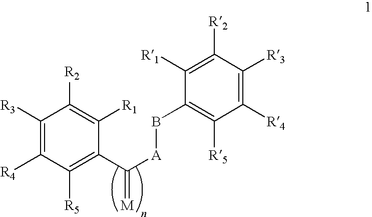

wherein, independently for each occurrence,

R₁, R₂, R₃; R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, aralkyl, —SO₃H, monosaccharide, oligosaccharide, glycofuranosyl, glycopyranosyl, glucuronosyl, or glucuronide;

M represents O, NR, or S;

A-B represents a bivalent alkyl, alkenyl, alkynyl, amido, sulfonamido, diazo, ether, alkylamino, alkylsulfide, hydroxylamine, or hydrazine group; and

n is 0 or 1.

In a further embodiment, a compound may be a compound of formula 1 and the attendant definitions, wherein n is 0. In a further embodiment, a compound may be a compound of formula 1 and the attendant definitions, wherein n is 1. In a further embodiment, a compound may be a compound of formula 1 and the attendant definitions, wherein A-B is ethenyl. In a further embodiment, a compound may be a compound of formula 1 and the attendant definitions, wherein A-B is —CH₂CH(Me)CH(Me)CH₂—. In a further embodiment, a compound may be a compound of formula 1 and the attendant definitions, wherein M is O. In a further embodiment, a compound may be a compound of formula 1 and the attendant definitions, wherein R₁, R₂, R₃, R₄, R₅, R′₁, R′₂, R′₃, R′₄, and R′₅ are H. In a further embodiment, a compound may be a compound of formula 1 and the attendant definitions, wherein R₂, R₄, and R′₃ are OH. In a further embodiment, a compound may be a compound of formula 1 and the attendant definitions, wherein R₂, R₄, R′₂ and R′₃ are OH. In a further embodiment, a compound may be a compound of formula 1 and the attendant definitions, wherein R₃, R₅, R′₂ and R′₃ are OH. In a further embodiment, a compound may be a compound of formula 1 and the attendant definitions, wherein R₁, R₃, R₅, R′₂ and R′₃ are OH. In a further embodiment, a compound may be a compound of formula 1 and the attendant definitions, wherein R₂ and R′₂ are OH; R₄ is O-β-D-glucoside; and R′₃ is OCH₃. In a further embodiment, a compound may be a compound of formula 1 and the attendant definitions, wherein R₂ is OH; R₄ is O-β-D-glucoside; and R′₃ is OCH₃.

In a further embodiment, a compound may be a compound of formula 1 and the attendant definitions, wherein n is 0; A-B is ethenyl; and R₁, R₂, R₃, R₄, R₅, R′₁, R′₂, R′₃, R′₄, and R′₅ are H (trans stilbene). In a further embodiment, a compound may be a compound of formula 1 and the attendant definitions, wherein n is 1; A-B is ethenyl; M is O; and R₁, R₂, R₃, R₄, R₅, R′₁, R′₂, R′₃, R′₄, and R′₅ are H (chalcone). In a further embodiment, a compound may be a compound of formula 1 and the attendant definitions, wherein n is 0; A-B is ethenyl; R₂, R₄, and R′₃ are OH; and R₁, R₃, R₅, R′₁, R′₂, R′4, and R′₅ are H (resveratrol). In a further embodiment, a compound may be a compound of formula 1 and the attendant definitions, wherein n is 0; A-B is ethenyl; R₂, R₄, R′₂ and R′₃ are OH; and R₁, R₃, R₅, R′₁, R′₄ and R′₅ are H (piceatannol). In a further embodiment, a compound may be a compound of formula 1 and the attendant definitions, wherein n is 1; A-B is ethenyl; M is O; R₃, R₅, R′₂ and R′₃ are OH; and R₁, R₂, R₄, R′₁, R′₄, and R′₅ are H (butein). In a further embodiment, a compound may be a compound of formula 1 and the attendant definitions, wherein n is 1; A-B is ethenyl; M is O; R₁, R₃, R₅, R′₂ and R′₃ are OH; and R₂, R₄, R′₁, R′₄, and R′₅ are H (3,4,2′,4′,6′-pentahydroxychalcone). In a further embodiment, a compound may be a compound of formula 1 and the attendant definitions, wherein n is 0; A-B is ethenyl; R₂ and R′₂ are OH, R₄ is O-β-D-glucoside, R′₃ is OCH₃; and R₁, R₃, R₅, R′₁, R′₄, and R′₅ are H (rhapontin). In a further embodiment, a compound may be a compound of formula 1 and the attendant definitions, wherein n is 0; A-B is ethenyl; R₂ is OH, R₄ is O-β-D-glucoside, R′₃ is OCH₃; and R₁, R₃, R₅, R′₁, R′₂, R′₄, and R′₅ are H (deoxyrhapontin). In a further embodiment, a compound may be a compound of formula 1 and the attendant definitions, wherein n is 0; A-B is —CH₂CH(Me)CH(Me)CH₂—; R₂, R₃, R′₂, and R′₃ are OH; and R₁, R₄, R₅, R′₁, R′₄, and R′₅ are H (NDGA).

A xenohormetic compound may also be a compound represented by formula 2 having xenohormetic activity:

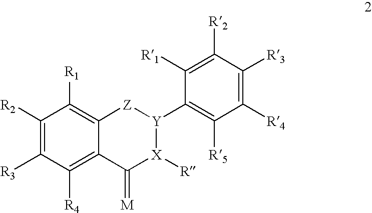

wherein, independently for each occurrence,

R₁, R₂, R₃, R₄, R′₁, R′₂, R′₃, R′₄, R′₅, and R″ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, aralkyl, —SO₃H, monosaccharide, oligosaccharide, glycopyranosyl, glycopyranosyl, glucuronosyl, or glucuronide;

M represents H₂, O, NR, or S;

Z represents CR, O, NR, or S;

X represents CR or N; and

Y represents CR or N.

In a further embodiment, the methods comprise a compound of formula 2 and the attendant definitions, wherein X and Y are both CH. In a further embodiment, the methods comprise a compound of formula 2 and the attendant definitions, wherein M is O. In a further embodiment, the methods comprise a compound of formula 2 and the attendant definitions, wherein M is H₂. In a further embodiment, the methods comprise a compound of formula 2 and the attendant definitions, wherein Z is O. In a further embodiment, the methods comprise a compound of formula 2 and the attendant definitions, wherein R″ is H. In a further embodiment, the methods comprise a compound of formula 2 and the attendant definitions, wherein R″ is OH. In a further embodiment, the methods comprise a compound of formula 2 and the attendant definitions, wherein R″ is an alkoxycarbonyl. In a further embodiment, the methods comprise a compound of formula 2 and the attendant definitions, wherein R₁ is

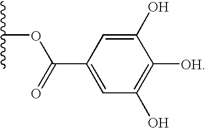

In a further embodiment, the methods comprise a compound of formula 2 and the attendant definitions, wherein R₁, R₂, R₃, R₄, R′₁, R′₂, R′₃, R′₄, R′₅ and R″ are H. In a further embodiment, the methods comprise a compound of formula 2 and the attendant definitions, wherein R₂, R₄, and R′₃ are OH. In a further embodiment, the methods comprise a compound of formula 2 and the attendant definitions, wherein R₄, R′₂, R′₃, and R″ are OH. In a further embodiment, the methods comprise a compound of formula 2 and the attendant definitions, wherein R₂, R₄, R′₂, R′₃, and R″ are OH. In a further embodiment, the methods comprise a compound of formula 2 and the attendant definitions, wherein R₂, R₄, R′₂, R′₃, R′₄, and R″ are OH.

In a further embodiment, the methods comprise a compound of formula 2 and the attendant definitions, wherein X and Y are CH; M is O; Z and O; R″ is H; and R₁, R₂, R₃, R₄, R′₁, R′₂, R′₃, R′₄, R′₅ and R″ are H (flavanone). In a further embodiment, the methods comprise a compound of formula 2 and the attendant definitions, wherein X and Y are CH; M is O; Z and O; R″ is H; R₂, R₄, and R′₃ are OH; and R₁, R₃, R′₁, R′₂, R′₄, and R′₅ are H (naringenin). In a further embodiment, the methods comprise a compound of formula 2 and the attendant definitions, wherein X and Y are CH; M is O; Z and O; R″ is OH; R₂, R₄, R′₂, and R′₃ are OH; and R₁, R₃, R′₁, R′₄, and R′₅ are H (3,5,7,3′,4′-pentahydroxyflavanone). In a further embodiment, the methods comprise a compound of formula 2 and the attendant definitions, wherein X and Y are CH; M is H₂; Z and O; R″ is OH; R₂, R₄, R′₂, and R′₃, are OH; and R₁, R₃, R′₁, R′₄ and R′₅ are H (epicatechin). In a further embodiment, the methods comprise a compound of formula 2 and the attendant definitions, wherein X and Y are CH; M is H₂; Z and O; R″ is OH; R₂, R₄, R′₂, R′₃, and R′₄ are OH; and R₁, R₃, R′1, and R′₅ are H (gallocatechin). In a further embodiment, the methods comprise a compound of formula 2 and the attendant definitions, wherein X and Y are CH; M is H₂; Z and O; R″ is

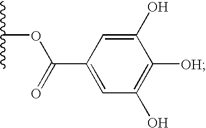

R₂, R₄, R′₂, R′₃, R′₄, and R″ are OH; and R₁, R₃, R′₁, and R′₅ are H (epigallocatechin gallate).

A xenohormetic compound may also be a compound represented by formula 3 having xenohormetic activity:

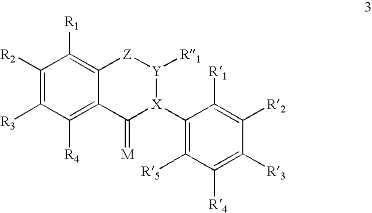

wherein, independently for each occurrence,

R₁, R₂, R₃, R₄, R′₁, R′₂, R′₃, R′₄, R′₅, and R″₁ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, aralkyl, —SO₃H, monosaccharide, oligosaccharide, glycofuranosyl, glycopyranosyl, glucuronosyl, or glucuronide;

M represents H₂, O, NR, or S;

Z represents C(R)₂, O, NR, or S;

X represents CR or N; and

Y represents CR or N.

A xenohormetic compound may also be a compound represented by formula 4 having xenohormetic activity:

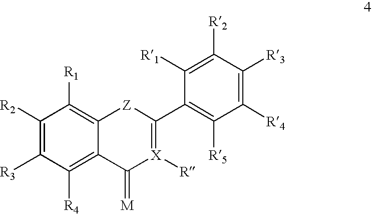

wherein, independently for each occurrence,

R₁, R₂, R₃, R₅, R′₁, R′₂, R′₃, R′₄, and R′₅, represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, aralkyl, —SO₃H, monosaccharide, oligosaccharide, glycofuranosyl, glycopyranosyl, glucuronosyl, or glucuronide;

M represents H₂, O, NR, or S;

Z represents CR, O, NR, or S; and

X represents CR″ or N, wherein

R″ is H, alkyl, aryl, heteroaryl, alkaryl, heteroaralkyl, halide, NO₂, SR, OR, N(R)₂, or carboxyl.

In a further embodiment, the methods comprise a compound of formula 4 and the attendant definitions, wherein X is C. In a further embodiment, the methods comprise a compound of formula 4 and the attendant definitions, wherein X is CR. In a further embodiment, the methods comprise a compound of formula 4 and the attendant definitions, wherein Z is O. In a further embodiment, the methods comprise a compound of formula 4 and the attendant definitions, wherein M is O. In a further embodiment, the methods comprise a compound of formula 4 and the attendant definitions, wherein R″ is H. In a further embodiment, the methods comprise a compound of formula 4 and the attendant definitions, wherein R″ is OH. In a further embodiment, the methods comprise a compound of formula 4 and the attendant definitions, wherein R₁, R₂, R₃, R₄, R′₁, R′₂, R′₃, R′₄, and R′₅ are H. In a further embodiment, the methods comprise a compound of formula 4 and the attendant definitions, wherein R₂, R′₂, and R′₃ are OH. In a further embodiment, the methods comprise a compound of formula 4 and the attendant definitions, wherein R₂, R₄, R′₂, R′₃, and R′₄ are OH. In a further embodiment, the methods comprise a compound of formula 4 and the attendant definitions, wherein R₂, R₄, R′₂, and R′₃ are OH. In a further embodiment, the methods comprise a compound of formula 4 and the attendant definitions, wherein R₃, R′₂, and R′₃ are OH. In a further embodiment, the methods comprise a compound of formula 4 and the attendant definitions, wherein R₂, R₄, R′₂, and R′₃ are OH. In a further embodiment, the methods comprise a compound of formula 4 and the attendant definitions, wherein R₂, R′₂, R′₃, and R′₄ are OH. In a further embodiment, the methods comprise a compound of formula 4 and the attendant definitions, wherein R₂, R₄, and R′₃ are OH. In a further embodiment, the methods comprise a compound of formula 4 and the attendant definitions, wherein R₂, R₃, R₄, and R′₃ are OH. In a further embodiment, the methods comprise a compound of formula 4 and the attendant definitions, wherein R₂, R₄, and R′₃ are OH. In a further embodiment, the methods comprise a compound of formula 4 and the attendant definitions, wherein R₃, R′₁, and R′₃ are OH. In a further embodiment, the methods comprise a compound of formula 4 and the attendant definitions, wherein R₂ and R′₃ are OH. In a further embodiment, the methods comprise a compound of formula 4 and the attendant definitions, wherein R₁, R₂, R′₂, and R′₃ are OH. In a further embodiment, the methods comprise a compound of formula 4 and the attendant definitions, wherein R₃, R′₁, and R′₂ are OH. In a further embodiment, the methods comprise a compound of formula 4 and the attendant definitions, wherein R′₃ is OH. In a further embodiment, the methods comprise a compound of formula 4 and the attendant definitions, wherein R₄ and R′₃ are OH. In a further embodiment, the methods comprise a compound of formula 4 and the attendant definitions, wherein R₂ and R₄ are OH. In a further embodiment, the methods comprise a compound of formula 4 and the attendant definitions, wherein R₂, R₄, R′₁, and R′₃ are OH. In a further embodiment, the methods comprise a compound of formula 4 and the attendant definitions, wherein R₄ is OH. In a further embodiment, the methods comprise a compound of formula 4 and the attendant definitions, wherein R₂, R₄, R′₂, R′₃, and R′₄ are OH. In a further embodiment, the methods comprise a compound of formula 4 and the attendant definitions, wherein R₂, R′₂, R′₃, and R′₄ are OH. In a further embodiment, the methods comprise a compound of formula 4 and the attendant definitions, wherein R₁, R₂, R₄, R′₂, and R′₃ are OH.

In a further embodiment, the methods comprise a compound of formula 4 and the attendant definitions, wherein X is CH; Z is O; M is O; and R₁, R₂, R₃, R₄, R′₁, R′₂, R′₃, R′₄, and R′₅ are H (flavone). In a further embodiment, the methods comprise a compound of formula 4 and the attendant definitions, wherein X is COH; Z is O; M is O; R₂, R′₂, and R′₃ are OH; and R₁, R₃, R₄, R′₁, R′₄, and R′₅ are H (fisetin). In a further embodiment, the methods comprise a compound of formula 4 and the attendant definitions, wherein X is CH; Z is O; M is O; R₂, R₄, R′₂, R′₃, and R′₄ are OH; and R₁, R₃, R′₁, and R′₅ are H (5,7,3′,4′,5′-pentahydroxyflavone). In a further embodiment, the methods comprise a compound of formula 4 and the attendant definitions, wherein X is CH; Z is O; M is O; R₂, R₄, R′₂, and R′₃ are OH; and R₁, R₃, R′₁, R′₄, and R′₅ are H (luteolin). In a further embodiment, the methods comprise a compound of formula 4 and the attendant definitions, wherein X is COH; Z is O; M is O; R₃, R′₂, and R′₃ are OH; and R₁, R₂, R₄, R′₁, R′₄, and R′₅ are H (3,6,3′,4′-tetrahydroxyflavone). In a further embodiment, the methods comprise a compound of formula 4 and the attendant definitions, wherein X is COH; Z is O; M is O; R₂, R₄, R′₂, and R′₃ are OH; and R₁, R₃, R′₁, R′₄, and R′₅ are H (quercetin). In a further embodiment, the methods comprise a compound of formula 4 and the attendant definitions, wherein X is CH; Z is O; M is O; R₂, R′₂, R′₃, and R′₄ are OH; and R₁, R₃, R₄, R′₁, and R′₅ are H. In a further embodiment, the methods comprise a compound of formula 4 and the attendant definitions, wherein X is COH; Z is O; M is O; R₂, R₄, and R′₃ are OH; and R₁, R₃, R′₁, R′₂, R′₄, and R′₅ are H. In a further embodiment, the methods comprise a compound of formula 4 and the attendant definitions, wherein X is CH; Z is O; M is O; R₂, R₃, R₄, and R′₃ are OH; and R₁, R′₁, R′₂, R′₄, and R′₅ are H. In a further embodiment, the methods comprise a compound of formula 4 and the attendant definitions, wherein X is CH; Z is O; M is O; R₂, R₄, and R′₃ are OH; and R₁, R₃, R′₁, R′₂, R′4, and R′₅ are H. In a further embodiment, the methods comprise a compound of formula 4 and the attendant definitions, wherein X is COH; Z is O; M is O; R₃, R′₁, and R′₃ are OH; and R₁, R₂, R₄, R′₂, R′₄, and R′₅ are H. In a further embodiment, the methods comprise a compound of formula 4 and the attendant definitions, wherein X is CH; Z is O; M is O; R₂ and R′₃ are OH; and R₁, R₃, R₄, R′₁, R′₂, R′₄, and R′ₛ are H. In a further embodiment, the methods comprise a compound of formula 4 and the attendant definitions, wherein X is COH; Z is O; M is O; R₁, R₂, R′₂, and R′₃ are OH; and R₁, R₂, R₄, R′₃, R′₄, and R′₅ are H. In a further embodiment, the methods comprise a compound of formula 4 and the attendant definitions, wherein X is COH; Z is O; M is O; R₃, R′₁, and R′₂ are OH; and R₁, R₂, R₄; R′₃, R′₄, and R′₅ are H. In a further embodiment, the methods comprise a compound of formula 4 and the attendant definitions, wherein X is CH; Z is O; M is O; R′₃ is OH; and R₁, R₂, R₃, R₄, R′₁, R′₂, R′₄, and R′₅ are H. In a further embodiment, the methods comprise a compound of formula 4 and the attendant definitions, wherein X is CH; Z is O; M is O; R₄ and R′₃ are OH; and R₁, R₂, R₃, R′₁, R′₂, R′₄, and R′ₛ are H. In a further embodiment, the methods comprise a compound of formula 4 and the attendant definitions, wherein X is CH; Z is O; M is O; R₂ and R₄ are OH; and R₁, R₃, R′₁, R′₂, R′₃, R′₄, and R′₅ are H. In a further embodiment, the methods comprise a compound of formula 4 and the attendant definitions, wherein X is COH; Z is O; M is O; R₂, R₄, R′₁, and R′₃ are OH; and R₁, R₃, R′₂, R′₄, and R′₅ are H. In a further embodiment, the methods comprise a compound of formula 4 and the attendant definitions, wherein X is CH; Z is O; M is O; R₄ is OH; and R₁, R₂, R₃, R′₁, R′₂, R′₃, R′₄, and R′ₛ are H. In a further embodiment, the methods comprise a compound of formula 4 and the attendant definitions, wherein X is COH; Z is O; M is O; R₂, R₄, R′₂, R′₃, and R′₄ are OH; and R₁, R₃, R′₁, and R′₅ are H. In a further embodiment, the methods comprise a compound of formula 4 and the attendant definitions, wherein X is COH; Z is O; M is O; R₂, R′₂, R′₃, and R′₄ are OH; and R₁, R₃, R₄, R′₁, and R′ₛ are H. In a further embodiment, the methods comprise a compound of formula 4 and the attendant definitions, wherein X is COH; Z is O; M is O; R₁, R₂, R₄, R′₂, and R′₃ are OH; and R₃, R′₁, R′₄, and R′₅ are H.

A xenohormetic compound may also be a compound represented by formula 5 having xenohormetic activity.

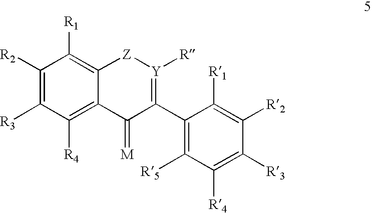

wherein, independently for each occurrence,

R₁, R₂, R₃, R₄, R′₁, R′₂, R′₃, R′₄, and R′₅, represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, aralkyl, —SO₃H, monosaccharide, oligosaccharide, glycofuranosyl, glycopyranosyl, glucuronosyl, or glucuronide;

M represents H₂, O, NR, or S;

Z represents C(R)₂, O, NR, or S; and

Y represents CR″ or N, wherein

R″ represents H, alkyl, aryl, heteroaryl, alkaryl, heteroaralkyl, halide, NO₂, SR, OR, N(R)₂, or carboxyl.

In a further embodiment, the methods comprise a compound of formula 5 and the attendant definitions, wherein Y is CR″. In a further embodiment, the methods comprise a compound of formula 5 and the attendant definitions, wherein Y is CH. In a further embodiment, the methods comprise a compound of formula 5 and the attendant definitions, wherein Z is O. In a further embodiment, the methods comprise a compound of formula 5 and the attendant definitions, wherein M is O. In a further embodiment, the methods comprise a compound of formula 5 and the attendant definitions, wherein R₂ and R′₃ are OH. In a further embodiment, the methods comprise a compound of formula 5 and the attendant definitions, wherein R₂, R₄, and R′₃ are OH.

In a further embodiment, the methods comprise a compound of formula 5 and the attendant definitions, wherein Y is CH; Z is O; M is O; R₂ and R′₃ are OH; and R₁, R₃, R₄; R′₁, R′₂, R′₄, and R′₅ are H. In a further embodiment, the methods comprise a compound of formula 5 and the attendant definitions, wherein Y is CH; Z is O; M is O; R₂, R₄, and R′₃ are OH; and R₁, R₃, R′₁, R′₂, R′₄, and R′₅ are H.

A xenohormetic compound may also be a compound represented by formula 6 having xenohormetic activity:

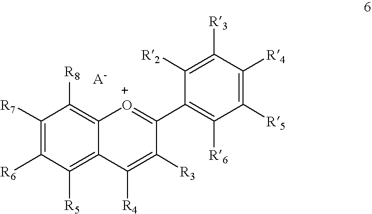

wherein, independently for each occurrence,

R₃, R₄, R₅, R₆, R₇, R₈, R′₂, R′₃, R′₄, R′₅, and R′₆ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, aralkyl, —SO₃H, monosaccharide, oligosaccharide, glycofuranosyl, glycopyranosyl, glucuronosyl, or glucuronide; and

A⁻ represents an anion selected from the following: Cl⁻, Br⁻, or I⁻.

In a further embodiment, the methods comprise a compound of formula 6 and the attendant definitions, wherein A⁻ is Cl⁻. In a further embodiment, the methods comprise a compound of formula 6 and the attendant definitions, wherein R₃, R₅, R₇, and R′₄ are OH.

In a further embodiment, the methods comprise a compound of formula 6 and the attendant definitions, wherein R₃, R₅, R₇, R′₃, and R′₄ are OH. In a further embodiment, the methods comprise a compound of formula 6 and the attendant definitions, wherein R₃, R₅, R₇, R′₃, R′4, and R′₅ are OH.

In a further embodiment, the methods comprise a compound of formula 6 and the attendant definitions, wherein A⁻ is Cr; R₃, R₅, R₇, and R′₄ are OH; and R₄, R₆, R₈, R′₂, R′₃, R′₅, and R′₆ are H. In a further embodiment, the methods comprise a compound of formula 6 and the attendant definitions, wherein A⁻ is Cl⁻; R₃, R₅, R₇, R′₃, and R′₄ are OH; and R₄, R₆, R₈, R′₂, R′₅, and R′₆ are H. In a further embodiment, the methods comprise a compound of formula 6 and the attendant definitions, wherein A⁻ is Cl⁻; R₃, R₅, R₇, R′₃, R′₄, and R′₅ are OH; and R₄, R₆, R₈, R′₂, and R′₆ are H.

A xenohormetic compound may also be a compound represented by formula 7 having xenohormetic activity:

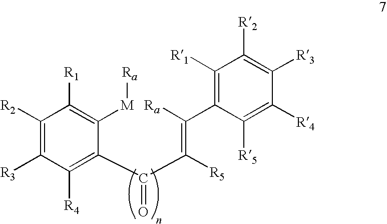

wherein, independently for each occurrence,

M is absent or O;

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

Rₐ represents H or the two instances of Rₐ form a bond;

R represents H, alkyl, aryl, heteroaryl, aralkyl, —SO3H, monosaccharide, oligosaccharide, glycofuranosyle, glycopyranosyl, glucuronosyl, or glucuronide; and

n is 0 or 1.

In a further embodiment, the methods comprise an activating compound represented by formula 7 and the attendant definitions, wherein n is 0. In a further embodiment, the methods comprise an activating compound represented by formula 7 and the attendant definitions, wherein n is 1. In a further embodiment, the methods comprise an activating compound represented by formula 7 and the attendant definitions, wherein M is absent. In a further embodiment, the methods comprise an activating compound represented by formula 7 and the attendant definitions, wherein M is O. In a further embodiment, the methods comprise an activating compound represented by formula 7 and the attendant definitions, wherein R₁ is H. In a further embodiment, the methods comprise an activating compound represented by formula 7 and the attendant definitions, wherein M is O and the two Rₐ form a bond.

In a further embodiment, the methods comprise an activating compound represented by formula 7 and the attendant definitions, wherein R₅ is H. In a further embodiment, the methods comprise an activating compound represented by formula 7 and the attendant definitions, wherein R₅ is OH. In a further embodiment, the methods comprise an activating compound represented by formula 7 and the attendant definitions, wherein R₁, R₃, and R′₃ are OH. In a further embodiment, the methods comprise an activating compound represented by formula 7 and the attendant definitions, wherein R₂, R₄, R′₂, and R′₃ are OH. In a further embodiment, the methods comprise an activating compound represented by formula 7 and the attendant definitions, wherein R₂, R′₂, and R′₃ are OH. In a further embodiment, the methods comprise an activating compound represented by formula 7 and the attendant definitions, wherein R₂ and R₄ are OH.

In a further embodiment, the methods include contacting a cell with an activating compound represented by formula 7 and the attendant definitions, wherein n is 0; M is absent; R₁ is H; R₅ is H; R₁, R₃, and R′₃ are OH; and R₂, R₄, R′₁, R′₂, R′₄, and R′₅ are H. In a further embodiment, the methods comprise an activating compound represented by formula 7 and the attendant definitions, wherein n is 1; M is absent; Rₐ is H; R₅ is H; R₂, R₄, R′₂, and R′₃ are OH; and R₁, R₃, R′₁, R′₄, and R′₅ are H. In a further embodiment, the methods comprise an activating compound represented by formula 7 and the attendant definitions, wherein n is 1; M is O; the two Rₐ form a bond; R₅ is OH; R₂, R′₂, and R′₃ are OH; and R₁, R₃, R₄, R′₁, R′₄, and R′₅ are H.

Other xenohormetic compounds may also include compounds having a formula selected from the group consisting of formulas 8-25 and 30 set forth below:

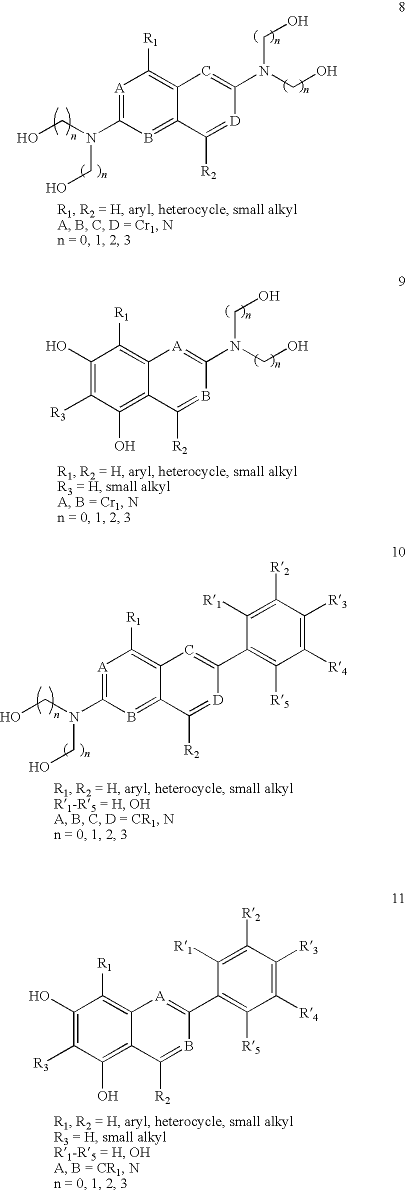

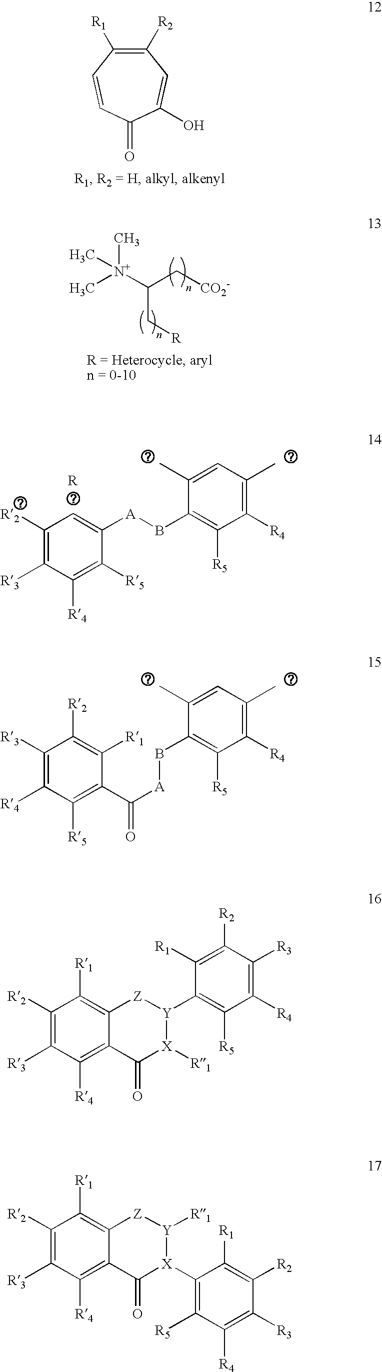

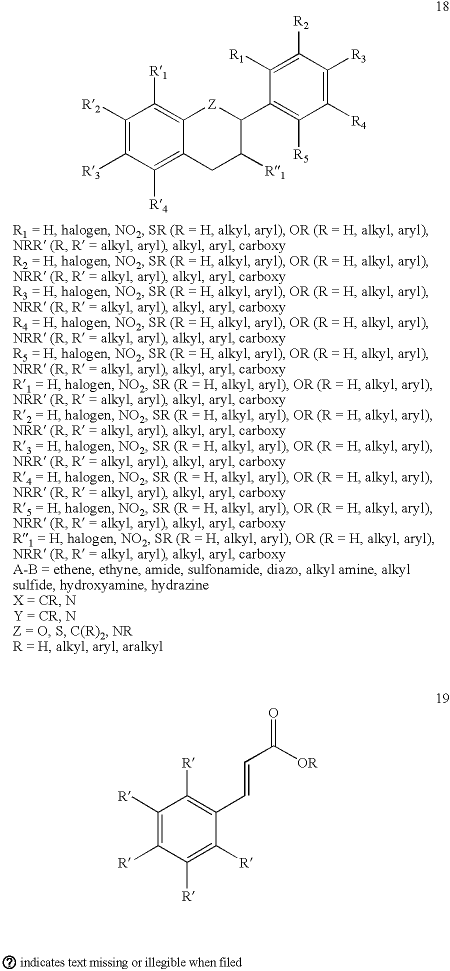

wherein, independently for each occurrence,

R=H, alkyl, aryl, heteroaryl, aralkyl, —SO₃H, monosaccharide, oligosaccharide, glycofuranosyl, glycopyranosyl, glucuronosyl, or glucuronide; and

R′=H, halogen, NO₂, SR, OR, NR₂, alkyl, aryl, or carboxy;

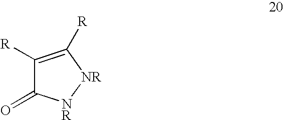

wherein, independently for each occurrence,

R=H, alkyl, aryl, heterocyclyl, heteroaryl, or aralkyl;

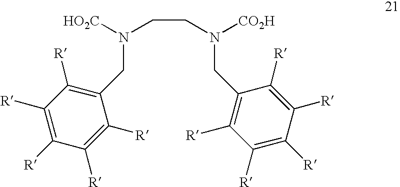

wherein, independently for each occurrence,

R′=H, halogen, NO₂, SR, OR, NR₂, alkyl, aryl, aralkyl, or carboxy; and

R=H, alkyl, aryl, heteroaryl, aralkyl, —SO₃H, monosaccharide, oligosaccharide, glycofuranosyl, glycopyranosyl, glucuronosyl, or glucuronide;

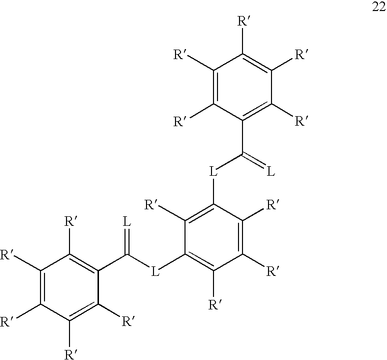

wherein, independently for each occurrence,

L represents CR₂, O, NR, or S;

R represents H, alkyl, aryl, heteroaryl, aralkyl, —SO₃H, monosaccharide, oligosaccharide, glycofuranosyl, glycopyranosyl, glucuronosyl, or glucuronide; and

R′ represents H, halogen, NO₂, SR, OR, NR₂, alkyl, aryl, aralkyl, or carboxy;

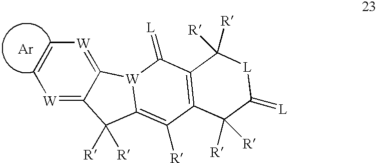

wherein, independently for each occurrence,

L represents CR₂, O, NR, or S;

W represents CR or N;

R represents H, alkyl, aryl, aralkyl, or heteroaralkyl;

Ar represents a fused aryl or heteroaryl ring; and

R′ represents H, halogen, NO₂, SR, OR, NR₂, alkyl, aryl, aralkyl, or carboxy;

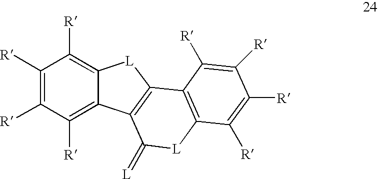

wherein, independently for each occurrence,

L represents CR₂, O, NR, or S;

R represents H, alkyl, aryl, heteroaryl, aralkyl, —SO₃H, monosaccharide, oligosaccharide, glycofuranosyl, glycopyranosyl, glucuronosyl, or glucuronide; and

R′ represents H, halogen, NO₂, SR, OR, NR₂, alkyl, aryl, aralkyl, or carboxy;

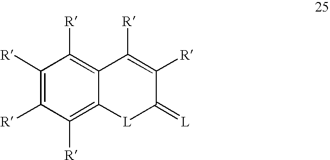

wherein, independently for each occurrence,

L represents CR₂, O, NR, or S;

R represents H, alkyl, aryl, heteroaryl, aralkyl, —SO₃H, monosaccharide, oligosaccharide, glycopyranosyl, glycopyranosyl, glucuronosyl, or glucuronide; and

R′ represents H, halogen, NO₂, SR, OR, NR₂, alkyl, aryl, aralkyl, or carboxy;

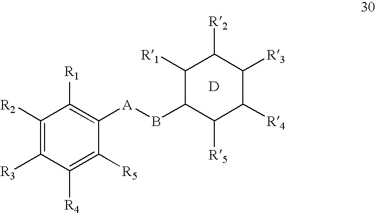

wherein, independently for each occurrence,

D is a phenyl or cyclohexyl group;

R₁, R₂, R₃, R₄, R₅, R′₁, R′₂, R′₃, R′₄, and R′₅ represent H, alkyl, aryl, heteroaryl, alkaryl, heteroaralkyl, halide, NO₂, SR, OR, N(R)₂, carboxyl, azide, ether; or any two adjacent R or R′ groups taken together form a fused benzene or cyclohexyl group;

R represents H, alkyl, aryl, heteroaryl, aralkyl, —SO₃H, monosaccharide, oligosaccharide, glycofuranosyl, glycopyranosyl, glucuronosyl, or glucuronide; and

A-B represents an ethylene, ethenylene, or imine group;

provided that when A-B is ethenylene, D is phenyl, and R′₃ is H: R₃ is not OH when R₁, R₂, R₄, and R₅ are H; and R₂ and R₄ are not OMe when R₁, R₃, and R₅ are H; and R₃ is not OMe when R₁, R₂, R₄, and R₅ are H.

In a further embodiment, the methods include contacting a cell with a xenohormetic compound represented by formula 30 and the attendant definitions, wherein D is a phenyl group.

In a further embodiment, the methods include contacting a cell with a xenohormetic compound represented by formula 30 and the attendant definitions, wherein A-B is an ethenylene or imine group.

In a further embodiment, the methods include contacting a cell with a xenohormetic compound represented by formula 30 and the attendant definitions, wherein A-B is an ethenylene group.

In a further embodiment, the methods include contacting a cell with a xenohormetic compound represented by formula 30 and the attendant definitions, wherein R₂ is OH.

In a further embodiment, the methods include contacting a cell with a xenohormetic compound represented by formula 30 and the attendant definitions, wherein R₄ is OH

In a further embodiment, the methods include contacting a cell with a xenohormetic compound represented by formula 30 and the attendant definitions, wherein R₂ and R₄ are OH.

In a further embodiment, the methods include contacting a cell with a xenohormetic compound represented by formula 30 and the attendant definitions, wherein D is a phenyl group; and A-B is an ethenylene group.

In a further embodiment, the methods include contacting a cell with a xenohormetic compound represented by formula 30 and the attendant definitions, wherein D is a phenyl group; A-B is an ethenylene group; and R₂ and R₄ are OH.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is Cl.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is OH.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is H.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is CH₂CH₃.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is F.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is Me.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is an azide.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is SMe.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is NO₂.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is CH(CH₃)₂.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is OMe.

In a further embodiment, the methods include contacting a cell with a xenohormetic compound represented by formula 30 and the attendant definitions, wherein A-B is ethenylene; D is a phenyl ring; R₂ and R₄ are OH; R′₂ is OH; and R′₃ is OMe.

In a further embodiment, the methods include contacting a cell with a xenohormetic compound represented by formula 30 and the attendant definitions, wherein A-B is ethenylene; D is a phenyl ring; R₂ is OH; R₄ is carboxyl; and R′₃ is OH.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is carboxyl.

In a further embodiment, the methods include contacting a cell with a xenohormetic compound represented by formula 30 and the attendant definitions, wherein A-B is ethenylene; D is a phenyl ring; R₂ and R₄ are OH; and R′₃ and R′₄ taken together form a fused benzene ring.

In a further embodiment, the methods include contacting a cell with a xenohormetic compound represented by formula 30 and the attendant definitions, wherein A-B is ethenylene; D is a phenyl ring; and R₄ is OH.

In a further embodiment, the methods include contacting a cell with a xenohormetic compound represented by formula 30 and the attendant definitions, wherein A-B is ethenylene; D is a phenyl ring; R₂ and R₄ are OCH₂OCH₃; and R′₃ is SMe.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is carboxyl.

In a further embodiment, the methods include contacting a cell with a xenohormetic compound represented by formula 30 and the attendant definitions, wherein A-B is ethenylene; D is a cyclohexyl ring; and R₂ and R₄ are OH.

In a further embodiment, the methods include contacting a cell with a xenohormetic compound represented by formula 30 and the attendant definitions, wherein A-B is ethenylene; D is a phenyl ring; and R₃ and R₄ are OMe.

In a further embodiment, the methods include contacting a cell with a xenohormetic compound represented by formula 30 and the attendant definitions, wherein A-B is ethenylene; D is a phenyl ring; R₂ and R₄ are OH; and R′₃ is OH.

In certain embodiments, a compound has a structure represented by formula 1-25, 30, and 32-65, with the proviso that the compound is not a specific compound, e.g., resveratrol or piceatannol.

In another embodiment, a xenohormetic compound may also be a compound represented by formula 32 having xenohormetic activity:

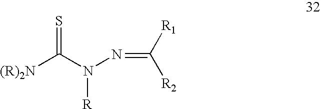

wherein, independently for each occurrence,

R is H, or a substituted or unsubstituted alkyl, aryl, aralkyl, heterocyclyl, heterocyclylalkyl, heteroaryl, or heteroaralkyl; and

R₁ and R₂ are a substituted or unsubstituted alkyl, aryl, aralkyl, heterocyclyl, heterocyclylalkyl, heteroaryl, or heteroaralkyl.

In a further embodiment, the methods comprise a compound of formula 32 and the attendant definitions wherein R is H.

In a further embodiment, the methods comprise a compound of formula 32 and the attendant definitions wherein R₁ is 3-hydroxyphenyl.

In a further embodiment, the methods comprise a compound of formula 32 and the attendant definitions wherein R₂ is methyl.

In a further embodiment, the methods comprise a compound of formula 32 and the attendant definitions wherein R is H and R₁ is 3-hydroxyphenyl.

In a further embodiment, the methods comprise a compound of formula 32 and the attendant definitions wherein R is H, R₁ is 3-hydroxyphenyl, and R₂ is methyl.

In another embodiment, a xenohormetic compound may also be a compound represented by formula 33 having xenohormetic activity:

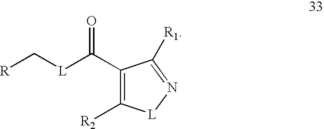

wherein, independently for each occurrence:

R is H, or a substituted or unsubstituted alkyl, alkenyl, or alkynyl;

R₁ and R₂ are a substituted or unsubstituted alkyl, aryl, aralkyl, heterocyclyl, heterocyclylalkyl, heteroaryl, or heteroaralkyl; and

L is O, S, or NR.

In a further embodiment, the methods comprise a compound of formula 33 and the attendant definitions wherein R is alkynyl.

In a further embodiment, the methods comprise a compound of formula 33 and the attendant definitions wherein R₁ is 2,6-dichlorophenyl.

In a further embodiment, the methods comprise a compound of formula 33 and the attendant definitions wherein R₂ is methyl.

In a further embodiment, the methods comprise a compound of formula 33 and the attendant definitions wherein L is O.

In a further embodiment, the methods comprise a compound of formula 33 and the attendant definitions wherein R is alkynyl and R₁ is 2,6-dichlorophenyl.

In a further embodiment, the methods comprise a compound of formula 33 and the attendant definitions wherein R is alkynyl, R₁ is 2,6-dichlorophenyl, and R₂ is methyl.

In a further embodiment, the methods comprise a compound of formula 33 and the attendant definitions wherein R is alkynyl, R₁ is 2,6-dichlorophenyl, R₂ is methyl, and L is O.

In another embodiment, a xenohormetic compound may also be a compound represented by formula 34 having xenohormetic activity:

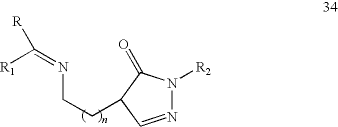

wherein, independently for each occurrence:

R, R₁, and R₂ are H, or a substituted or unsubstituted alkyl, aryl, aralkyl, heterocyclyl, heterocyclylalkyl, heteroaryl, or heteroaralkyl; and

n is an integer from 0 to 5 inclusive.

In a further embodiment, the methods comprise a compound of formula 34 and the attendant definitions wherein R is 3,5-dichloro-2-hydroxyphenyl.

In a further embodiment, the methods comprise a compound of formula 34 and the attendant definitions wherein R₁ is H.

In a further embodiment, the methods comprise a compound of formula 34 and the attendant definitions wherein R₂ is H.

In a further embodiment, the methods comprise a compound of formula 34 and the attendant definitions wherein n is 1.

In a further embodiment, the methods comprise a compound of formula 34 and the attendant definitions wherein R is 3,5-dichloro-2-hydroxyphenyl and R₁ is H.

In a further embodiment, the methods comprise a compound of formula 34 and the attendant definitions wherein R is 3,5-dichloro-2-hydroxyphenyl, R₁ is H, and R₂ is H.

In a further embodiment, the methods comprise a compound of formula 34 and the attendant definitions wherein R is 3,5-dichloro-2-hydroxyphenyl, R₁ is H, R₂ is H, and n is 1.

In another embodiment, a xenohormetic compound may also be a compound represented by formula 35 having xenohormetic activity:

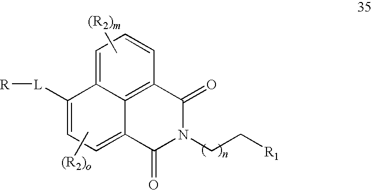

wherein, independently for each occurrence:

R is H or a substituted or unsubstituted alkyl, aryl, aralkyl, heterocyclyl, heterocyclylalkyl, heteroaryl, or heteroaralkyl;

R₁ is a substituted or unsubstituted alkyl, aryl, aralkyl, heterocyclyl, heterocyclylalkyl, heteroaryl, or heteroaralkyl;

R₂ is hydroxy, amino, cyano, halide, OR₃, ether, ester, amido, ketone, carboxylic acid, nitro, or a substituted or unsubstituted alkyl, aryl, aralkyl, heterocyclyl, heterocyclylalkyl, heteroaryl, heteroaralkyl;

R₃ is alkyl, —SO₃H, monosaccharide, oligosaccharide, glycofuranosyl, glycopyranosyl, glucuronosyl, or glucuronide;

L is O, NR, or S;

m is an integer from 0 to 3 inclusive;

n is an integer from 0 to 5 inclusive; and

o is an integer from 0 to 2 inclusive.

In a further embodiment, the methods comprise a compound of formula 35 and the attendant definitions wherein R is phenyl.

In a further embodiment, the methods comprise a compound of formula 35 and the attendant definitions wherein R₁ is pyridine.

In a further embodiment, the methods comprise a compound of formula 35 and the attendant definitions wherein L is S.

In a further embodiment, the methods comprise a compound of formula 35 and the attendant definitions wherein m is 0.

In a further embodiment, the methods comprise a compound of formula 35 and the attendant definitions wherein n is 1.

In a further embodiment, the methods comprise a compound of formula 35 and the attendant definitions wherein o is 0.

In a further embodiment, the methods comprise a compound of formula 35 and the attendant definitions wherein R is phenyl and R₁ is pyridine.

In a further embodiment, the methods comprise a compound of formula 35 and the attendant definitions wherein R is phenyl, R₁ is pyridine, and L is S.

In a further embodiment, the methods comprise a compound of formula 35 and the attendant definitions wherein R is phenyl, R₁ is pyridine, L is S, and m is 0.

In a further embodiment, the methods comprise a compound of formula 35 and the attendant definitions wherein R is phenyl, R₁ is pyridine, L is S, m is 0, and n is 1.

In a further embodiment, the methods comprise a compound of formula 35 and the attendant definitions wherein R is phenyl, R₁ is pyridine, L is S, m is 0, n is 1, and o is 0.

In another embodiment, a xenohormetic compound may also be a compound represented by formula 36 having xenohormetic activity:

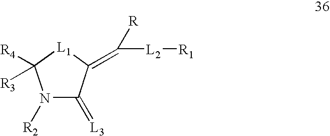

wherein, independently for each occurrence:

R, R₃, and R₄ are H, hydroxy, amino, cyano, halide, OR₅, ether, ester, amido, ketone, carboxylic acid, nitro, or a substituted or unsubstituted alkyl, aryl, aralkyl, heterocyclyl, heterocyclylalkyl, heteroaryl, heteroaralkyl;

R₅ is alkyl, —SO₃H, monosaccharide, oligosaccharide, glycofuranosyl, glycopyranosyl, glucuronosyl, or glucuronide;

R₁ and R₂ are H or a substituted or unsubstituted alkyl, aryl, aralkyl, heterocyclyl, heterocyclylalkyl, heteroaryl, heteroaralkyl;

L₁ is O, NR₁, S, C(R)₂, or SO₂; and

L₂ and L₃ are O, NR₁, S, or C(R)₂.

In a further embodiment, the methods comprise a compound of formula 36 and the attendant definitions wherein R is H.

In a further embodiment, the methods comprise a compound of formula 36 and the attendant definitions wherein R₁ is 4-chlorophenyl.

In a further embodiment, the methods comprise a compound of formula 36 and the attendant definitions wherein R₂ is 4-chlorophenyl.

In a further embodiment, the methods comprise a compound of formula 36 and the attendant definitions wherein R₃ is H.

In a further embodiment, the methods comprise a compound of formula 36 and the attendant definitions wherein R₄ is H.

In a further embodiment, the methods comprise a compound of formula 36 and the attendant definitions wherein L₁ is SO₂.

In a further embodiment, the methods comprise a compound of formula 36 and the attendant definitions wherein L₂ is NH.

In a further embodiment, the methods comprise a compound of formula 36 and the attendant definitions wherein L₃ is O.

In a further embodiment, the methods comprise a compound of formula 36 and the attendant definitions wherein R is H and R₁ is 4-chlorophenyl.

In a further embodiment, the methods comprise a compound of formula 36 and the attendant definitions wherein R is H, R₁ is 4-chlorophenyl, and R₂ is 4-chlorophenyl.

In a further embodiment, the methods comprise a compound of formula 36 and the attendant definitions wherein R is H, R₁ is 4-chlorophenyl, R₂ is 4-chlorophenyl, and R₃ is H.

In a further embodiment, the methods comprise a compound of formula 36 and the attendant definitions wherein R is H, R₁ is 4-chlorophenyl, R₂ is 4-chlorophenyl, R₃ is H, and R₄ is H.

In a further embodiment, the methods comprise a compound of formula 36 and the attendant definitions wherein R is H, R₁ is 4-chlorophenyl, R₂ is 4-chlorophenyl, R₃ is H, R₄ is H, and L₁ is SO₂.

In a further embodiment, the methods comprise a compound of formula 36 and the attendant definitions wherein R is H, R₁ is 4-chlorophenyl, R₂ is 4-chlorophenyl, R₃ is H, R₄ is H, L₁ is SO₂, and L₂ is NH.

In a further embodiment, the methods comprise a compound of formula 36 and the attendant definitions wherein R is H, R₁ is 4-chlorophenyl, R₂ is 4-chlorophenyl, R₃ is H, R₄ is H, L₁ is SO₂, L₂ is NH, and L₃ is O.

In another embodiment, a xenohormetic compound may also be a compound represented by formula 37 having xenohormetic activity:

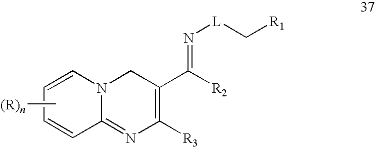

wherein, independently for each occurrence:

R is hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, heteroaralkyl;

R₁ is H or a substituted or unsubstituted alkyl, aryl, aralkyl, heterocyclyl, heterocyclylalkyl, heteroaryl, heteroaralkyl;

R₂ and R₃ are H or a substituted or unsubstituted alkyl, aryl, aralkyl, heterocyclyl, heterocyclylalkyl, heteroaryl, heteroaralkyl;

R₄ is alkyl, —SO₃H, monosaccharide, oligosaccharide, glycofuranosyl, glycopyranosyl, glucuronosyl, or glucuronide;

L is O, NR₁, or S; and

n is an integer from 0 to 4 inclusive.

In a further embodiment, the methods comprise a compound of formula 37 and the attendant definitions wherein R is methyl.

In a further embodiment, the methods comprise a compound of formula 37 and the attendant definitions wherein n is 1.

In a further embodiment, the methods comprise a compound of formula 37 and the attendant definitions wherein R₁ is 3-fluorophenyl.

In a further embodiment, the methods comprise a compound of formula 37 and the attendant definitions wherein R₂ is H.

In a further embodiment, the methods comprise a compound of formula 37 and the attendant definitions wherein R₃ is 4-chlorophenyl.

In a further embodiment, the methods comprise a compound of formula 37 and the attendant definitions wherein L is O.

In a further embodiment, the methods comprise a compound of formula 37 and the attendant definitions wherein R is methyl and n is 1.

In a further embodiment, the methods comprise a compound of formula 37 and the attendant definitions wherein R is methyl, n is 1, and R₁ is 3-fluorophenyl.

In a further embodiment, the methods comprise a compound of formula 37 and the attendant definitions wherein R is methyl, n is 1, R₁ is 3-fluorophenyl, and R₂ is H.

In a further embodiment, the methods comprise a compound of formula 37 and the attendant definitions wherein R is methyl, n is 1, R₁ is 3-fluorophenyl, R₂ is H, and R₃ is 4-chlorophenyl.

In another embodiment, a xenohormetic compound may also be a compound represented by formula 38 having xenohormetic activity:

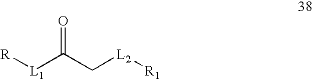

wherein, independently for each occurrence:

R and R₁ are H or a substituted or unsubstituted alkyl, aryl, aralkyl, heterocyclyl, heterocyclylalkyl, heteroaryl, or heteroaralkyl; and

L₁ and L₂ are O, NR, or S.

In a further embodiment, the methods comprise a compound of formula 38 and the attendant definitions wherein R is 3-methoxyphenyl.

In a further embodiment, the methods comprise a compound of formula 38 and the attendant definitions wherein R₁ is 4-t-butylphenyl.

In a further embodiment, the methods comprise a compound of formula 38 and the attendant definitions wherein L₁ is NH.

In a further embodiment, the methods comprise a compound of formula 38 and the attendant definitions wherein L₂ is O.

In a further embodiment, the methods comprise a compound of formula 38 and the attendant definitions wherein R is 3-methoxyphenyl and R₁ is 4-t-butylphenyl.

In a further embodiment, the methods comprise a compound of formula 38 and the attendant definitions wherein R is 3-methoxyphenyl, R₁ is 4-t-butylphenyl, and L₁ is NH.

In a further embodiment, the methods comprise a compound of formula 38 and the attendant definitions wherein R is 3-methoxyphenyl, R₁ is 4-t-butylphenyl, L₁ is NH, and L₂ is O.

In another embodiment, a xenohormetic compound may also be a compound represented by formula 39 having xenohormetic activity:

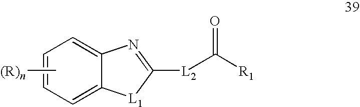

wherein, independently for each occurrence.

R is H, hydroxy, amino, cyano, halide, OR₂, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ is H or a substituted or unsubstituted alkyl, aryl, alkaryl, heterocyclyl, heterocyclylalkyl, heteroaryl, or heteroaralkyl;

R₂ is alkyl, —SO₃H, monosaccharide, oligosaccharide, glycofuranosyl, glycopyranosyl, glucuronosyl, or glucuronide;

L₁ and L₂ are O, NR, or S; and

n is an integer from 0 to 4 inclusive.

In a further embodiment, the methods comprise a compound of formula 39 and the attendant definitions wherein R is methyl.

In a further embodiment, the methods comprise a compound of formula 39 and the attendant definitions wherein n is 1.

In a further embodiment, the methods comprise a compound of formula 39 and the attendant definitions wherein R₁ is 3,4,5-trimethoxyphenyl.

In a further embodiment, the methods comprise a compound of formula 39 and the attendant definitions wherein L₁ is S.

In a further embodiment, the methods comprise a compound of formula 39 and the attendant definitions wherein L_{Z} is NH.

In a further embodiment, the methods comprise a compound of formula 39 and the attendant definitions wherein R is methyl and n is 1.

In a further embodiment, the methods comprise a compound of formula 39 and the attendant definitions wherein R is methyl, n is 1, and R₁ is 3,4,5-trimethoxyphenyl.

In a further embodiment, the methods comprise a compound of formula 39 and the attendant definitions wherein R is methyl, n is 1, R₁ is 3,4,5-trimethoxyphenyl, and L₁ is S.

In a further embodiment, the methods comprise a compound of formula 39 and the attendant definitions wherein R is methyl, n is 1, R₁ is 3,4,5-trimethoxyphenyl, L₁ is S, and L₂ is NH.

In another embodiment, a xenohormetic compound may also be a compound represented by formula 40 having xenohormetic activity:

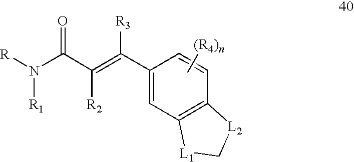

wherein, independently for each occurrence:

R, R₁, R₂, R₃ are H or a substituted or unsubstituted alkyl, aryl, alkaryl, heterocyclyl, heterocyclylalkyl, heteroaryl, or heteroaralkyl;

R₄ is hydroxy, amino, cyano, halide, OR₅, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₅ is alkyl, —SO₃H, monosaccharide, oligosaccharide, glycofuranosyl, glycopyranosyl, glucuronosyl, or glucuronide;

L₁ and L₂ are O, NR, or S; and

n is an integer from 0 to 3 inclusive.

In a further embodiment, the methods comprise a compound of formula 40 and the attendant definitions wherein R is H.

In a further embodiment, the methods comprise a compound of formula 40 and the attendant definitions wherein R₁ is perfluorophenyl.

In a further embodiment, the methods comprise a compound of formula 40 and the attendant definitions wherein R₂ is H.

In a further embodiment, the methods comprise a compound of formula 40 and the attendant definitions wherein R₃ is H.

In a further embodiment, the methods comprise a compound of formula 40 and the attendant definitions wherein L₁ is O.

In a further embodiment, the methods comprise a compound of formula 40 and the attendant definitions wherein L₂ is O.

In a further embodiment, the methods comprise a compound of formula 40 and the attendant definitions wherein n is O.

In a further embodiment, the methods comprise a compound of formula 40 and the attendant definitions wherein R is H and R₁ is perfluorophenyl.

In a further embodiment, the methods comprise a compound of formula 40 and the attendant definitions wherein R is H, R₁ is perfluorophenyl, and R₂ is H.

In a further embodiment, the methods comprise a compound of formula 40 and the attendant definitions R is H, R₁ is perfluorophenyl, R₂ is H, and R₃ is H.

In a further embodiment, the methods comprise a compound of formula 40 and the attendant definitions wherein R is H, R₁ is perfluorophenyl, R₂ is H, R₃ is H, and L₁ is O.

In a further embodiment, the methods comprise a compound of formula 40 and the attendant definitions wherein R is H, R₁ is perfluorophenyl, R₂ is H, R₃ is H, L₁ is O, and L₂ is O.

In a further embodiment, the methods comprise a compound of formula 40 and the attendant definitions wherein R is H, R₁ is perfluorophenyl, R₂ is H, R₃ is H, L₁ is O, L₂ is O, and n is 0.

In another embodiment, a xenohormetic compound may also be a compound represented by formula 41 having xenohormetic activity:

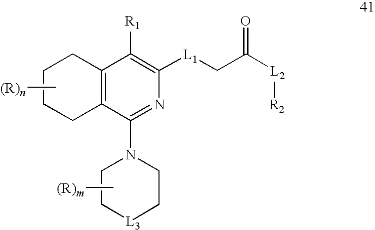

wherein, independently for each occurrence:

R, R₁, and R₃ are hydroxy, amino, cyano, halide, OR₄, ether, ester, amido, ketone, carboxylic acid, nitro, or a substituted or =substituted alkyl, aryl, aralkyl, heterocyclyl, heterocyclylalkyl, heteroaryl, or heteroaralkyl;

R₂ is H or a substituted or =substituted alkyl, aryl, aralkyl, heterocyclyl, heterocyclylalkyl, heteroaryl, or heteroaralkyl;

R₄ is alkyl, —SO₃H, monosaccharide, oligosaccharide, glycopyranosyl, glycopyranosyl, glucuronosyl, or glucuronide;

L₁, L₂, and L₃ are O, NR₂, or S; and

m and n are integers from 0 to 8 inclusive.

In a further embodiment, the methods comprise a compound of formula 41 and the attendant definitions wherein n is 0.

In a further embodiment, the methods comprise a compound of formula 41 and the attendant definitions wherein R₁ is cyano.

In a further embodiment, the methods comprise a compound of formula 41 and the attendant definitions wherein R₂ is ethyl.

In a further embodiment, the methods comprise a compound of formula 41 and the attendant definitions wherein m is 0.

In a further embodiment, the methods comprise a compound of formula 41 and the attendant definitions wherein L₁ is S.

In a further embodiment, the methods comprise a compound of formula 41 and the attendant definitions wherein L₂ is O.

In a further embodiment, the methods comprise a compound of formula 41 and the attendant definitions wherein L₃ is O.

In a further embodiment, the methods comprise a compound of formula 41 and the attendant definitions wherein n is 0 and R₁ is cyano.

In a further embodiment, the methods comprise a compound of formula 41 and the attendant definitions wherein n is 0, R₁ is cyano, and R₂ is ethyl.

In a further embodiment, the methods comprise a compound of formula 41 and the attendant definitions wherein n is 0, R₁ is cyano, R₂ is ethyl, and m is 0.

In a further embodiment, the methods comprise a compound of formula 41 and the attendant definitions wherein n is 0, R₁ is cyano, R₂ is ethyl, m is 0, and L₁ is S.

In a further embodiment, the methods comprise a compound of formula 41 and the attendant definitions wherein n is 0, R₁ is cyano, R₂ is ethyl, m is 0, L₁ is S, and L₂ is O.

In a further embodiment, the methods comprise a compound of formula 41 and the attendant definitions wherein n is 0, R₁ is cyano, R₂ is ethyl, m is 0, L₁ is S, L₂ is O, and L₃ is O.

In another embodiment, a xenohormetic compound may also be a compound represented by formula 42 having xenohormetic activity:

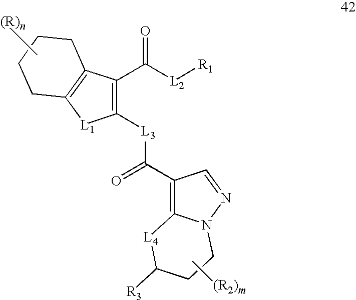

wherein, independently for each occurrence:

R and R₂ are H, hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₃ are H or a substituted or unsubstituted alkyl, aryl, aralkyl, heterocyclyl, heterocyclylalkyl, heteroaryl, or heteroaralkyl;

R₄ is alkyl, —SO₃H, monosaccharide, oligosaccharide, glycofuranosyl, glycopyranosyl, glucuronosyl, or glucuronide;

L₁, L₂, L₃, and L₄ are O, NR₁, or S;

m is an integer from 0 to 6 inclusive; and

n is an integer from 0 to 8 inclusive.

In a further embodiment, the methods comprise a compound of formula 42 and the attendant definitions wherein n is 0.

In a further embodiment, the methods comprise a compound of formula 42 and the attendant definitions wherein R₁ is methyl.

In a further embodiment, the methods comprise a compound of formula 42 and the attendant definitions wherein R₂ is CF₃ and m is 1.

In a further embodiment, the methods comprise a compound of formula 42 and the attendant definitions wherein R₃ is 4-methylphenyl.

In a further embodiment, the methods comprise a compound of formula 42 and the attendant definitions wherein L₁ is S.

In a further embodiment, the methods comprise a compound of formula 42 and the attendant definitions wherein L₂ is O.

In a further embodiment, the methods comprise a compound of formula 42 and the attendant definitions wherein L₃ is NR₁.

In a further embodiment, the methods comprise a compound of formula 42 and the attendant definitions wherein L₄ is NR₁.

In a further embodiment, the methods comprise a compound of formula 42 and the attendant definitions wherein n is 0 and R₁ is methyl.

In a further embodiment, the methods comprise a compound of formula 42 and the attendant definitions wherein n is 0, R₁ is methyl, R₂ is CF₃, and m is 1.

In a further embodiment, the methods comprise a compound of formula 42 and the attendant definitions wherein n is 0, R₁ is methyl, R₂ is CF₃, m is 1; and R₃ is 4-methylphenyl.

In a further embodiment, the methods comprise a compound of formula 42 and the attendant definitions wherein n is 0, R₁ is methyl, R₂ is CF₃, m is 1; R₃ is 4-methylphenyl; and L₁ is S.

In a further embodiment, the methods comprise a compound of formula 42 and the attendant definition wherein n is 0, R₁ is methyl, R₂ is CF₃, m is 1; R₃ is 4-methylphenyl; L₁ is S, and L₂ is O.

In a further embodiment, the methods comprise a compound of formula 42 and the attendant definitions wherein n is 0, R₁ is methyl, R₂ is CF₃, m is 1; R₃ is 4-methylphenyl; L₁ is S, L₂ is O; and L₃ is NR₁.

In a further embodiment, the methods comprise a compound of formula 42 and the attendant definitions wherein n is 0, R₁ is methyl, R₂ is CF₃, m is 1; R₃ is 4-methylphenyl; L₁ is S, L₂ is O; L₃ is NR₁, and L₄ is NR₁.

In another embodiment, a xenohormetic compound may also be a compound represented by formula 43 having xenohormetic activity:

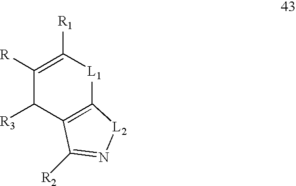

wherein, independently for each occurrence:

R and R₁ are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ and R₃ are H or a substituted or unsubstituted alkyl, aryl, aralkyl, heterocyclyl, heterocyclylalkyl, heteroaryl, or heteroaralkyl;

R₄ is alkyl, —SO₃H, monosaccharide, oligosaccharide, glycofuranosyl, glycopyranosyl, glucuronosyl, or glucuronide; and

L₁ and L₂ are O, NR₂, or S.

In a further embodiment, the methods comprise a compound of formula 43 and the attendant definitions wherein R₁ is cyano.

In a further embodiment, the methods comprise a compound of formula 43 and the attendant definitions wherein R₁ is NH₂.

In a further embodiment, the methods comprise a compound of formula 43 and the attendant definitions wherein R₂ is 4-bromophenyl.

In a further embodiment, the methods comprise a compound of formula 43 and the attendant definitions wherein R₃ is 3-hydroxy-4-methoxyphenyl.

In a further embodiment, the methods comprise a compound of formula 43 and the attendant definitions wherein L₁ is O.

In a further embodiment, the methods comprise a compound of formula 43 and the attendant definitions wherein L₂ is NR₂.

In a further embodiment, the methods comprise a compound of formula 43 and the attendant definitions wherein R is cyano and R₁ is NH₂.

In a further embodiment, the methods comprise a compound of formula 43 and the attendant definitions wherein R is cyano, R₁ is NH₂, and R₂ is 4-bromophenyl.

In a further embodiment, the methods comprise a compound of formula 43 and the attendant definitions wherein R is cyano, R₁ is NH₂, R₂ is 4-bromophenyl, and R₃ is 3-hydroxy-4-methoxyphenyl.

In a further embodiment, the methods comprise a compound of formula 43 and the attendant definitions wherein R is cyano, R₁ is NH₂, R₂ is 4-bromophenyl, R₃ is 3-hydroxy-4-methoxyphenyl, and L₁ is O.

In a further embodiment, the methods comprise a compound of formula 43 and the attendant definitions wherein R is cyano, R₁ is NH₂, R₂ is 4-bromophenyl, R₃ is 3-hydroxy-4-methoxyphenyl, L₁ is O, and L₂ is NR₂.

In another embodiment, a xenohormetic compound may also be a compound represented by formula 44 having xenohormetic activity:

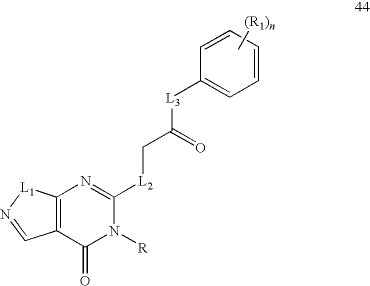

wherein, independently for each occurrence:

R is H or a substituted or unsubstituted alkyl, aryl, aralkyl, heterocyclyl, heterocyclylalkyl, heteroaryl, or heteroaralkyl;

R₁ is hydroxy, amino, cyano, halide, OR₂, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is alkyl, —SO₃H, monosaccharide, oligosaccharide, glycofuranosyl, glycopyranosyl, glucuronosyl, or glucuronide;

L₁, L₂, and L₃ are O, NR, or S; and

n is an integer from 0 to 5 inclusive.

In a further embodiment, the methods comprise a compound of formula 44 and the attendant definitions wherein R is 3-trifluoromethylphenyl.

In a further embodiment, the methods comprise a compound of formula 44 and the attendant definitions wherein R₁ is C(O)OCH₃.

In a further embodiment, the methods comprise a compound of formula 44 and the attendant definitions wherein L₁ is NR.

In a further embodiment, the methods comprise a compound of formula 44 and the attendant definitions wherein L₂ is S.

In a further embodiment, the methods comprise a compound of formula 44 and the attendant definitions wherein L₃ is NR.

In a further embodiment, the methods comprise a compound of formula 44 and the attendant definitions wherein n is 2.

In a further embodiment, the methods comprise a compound of formula 44 and the attendant definitions wherein R is 3-trifluoromethylphenyl and R₁ is C(O)OCH₃.

In a further embodiment, the methods comprise a compound of formula 44 and the attendant definitions wherein R is 3-trifluoromethylphenyl, R₁ is C(O)OCH₃, and L₁ is NR.

In a further embodiment, the methods comprise a compound of formula 44 and the attendant definitions wherein R is 3-trifluoromethylphenyl, R₁ is C(O)OCH₃, L₁ is NR, and L₂ is S.

In a further embodiment, the methods comprise a compound of formula 44 and the attendant definitions wherein R is 3-trifluoromethylphenyl, R₁ is C(O)OCH₃, Lᵢ is NR, L₂ is S, and L₃ is NR.

In a further embodiment, the methods comprise a compound of formula 44 and the attendant definitions wherein R is 3-trifluoromethylphenyl, R₁ is C(O)OCH₃, L₁ is NR, L₂ is S, L₃ is NR, and n is 2.

In another embodiment, a xenohormetic compound may also be a compound represented by formula 45 having xenohormetic activity:

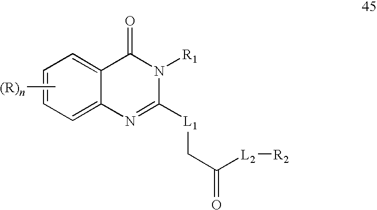

wherein, independently for each occurrence:

R is hydroxy, amino, cyano, halide, OR₃, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₂ are H or a substituted or unsubstituted alkyl, aryl, aralkyl, heterocyclyl, heterocyclylalkyl, heteroaryl, or heteroaralkyl;

R₃ is alkyl, —SO₃H, monosaccharide, oligosaccharide, glycofuranosyl, glycopyranosyl, glucuronosyl, or glucuronide;

L₁ and L₂ are O, NR₁, or S; and

n is an integer from 0 to 4 inclusive.

In a further embodiment, the methods comprise a compound of formula 45 and the attendant definitions wherein n is 0.

In a further embodiment, the methods comprise a compound of formula 45 and the attendant definitions wherein R₁ is 2-tetrahydrofuranylmethyl.

In a further embodiment, the methods comprise a compound of formula 45 and the attendant definitions wherein R₂ is —CH₂CH₂C₆H₄SO₂NH₂.

In a further embodiment, the methods comprise a compound of formula 45 and the attendant definitions wherein L₁ is S.

In a further embodiment, the methods comprise a compound of formula 45 and the attendant definitions wherein L₂ is NR₁.

In a further embodiment, the methods comprise a compound of formula 45 and the attendant definitions wherein n is 0 and R₁ is 2-tetrahydrofuranylmethyl.

In a further embodiment, the methods comprise a compound of formula 45 and the attendant definitions wherein n is 0, R₁ is 2-tetrahydrofuranylmethyl, and R₂ is —CH₂CH₂C₆H₄SO₂NH₂.

In a further embodiment, the methods comprise a compound of formula 45 and the attendant definitions wherein n is O, R₁ is 2-tetrahydrofuranylmethyl, R₂ is —CH₂CH₂C₆H₄SO₂NH₂, and L₁ is S.

In a further embodiment, the methods comprise a compound of formula 45 and the attendant definitions wherein n is 0, R₁ is 2-tetrahydrofuranylmethyl, R₂ is —CH₂CH₂C₆H₄SO₂NH₂, L₁ is S, and L₂ is NR₁.

In another embodiment, a xenohormetic compound may also be a compound represented by formula 46 having xenohormetic activity:

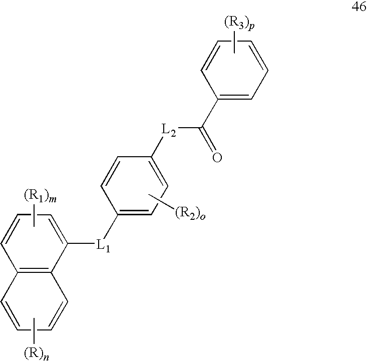

wherein, independently for each occurrence:

R, R₁, R₂, and R₃ are hydroxy, amino, cyano, halide, OR₅, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₅ is alkyl, —SO₃H, monosaccharide, oligosaccharide, glycofuranosyl, glycopyranosyl, glucuronosyl, or glucuronide;

L₁ and L₂ are O, NR₁, or S;

R₄ is H or a substituted or unsubstituted alkyl, aryl, aralkyl, heterocyclyl, heterocyclylalkyl, heteroaryl, or heteroaralkyl;

n is an integer from 0 to 4 inclusive;

m is an integer from 0 to 3 inclusive;

o is an integer from 0 to 4 inclusive; and

p is an integer from 0 to 5 inclusive.

In a further embodiment, the methods comprise a compound of formula 46 and the attendant definitions wherein n is 0.

In a further embodiment, the methods comprise a compound of formula 46 and the attendant definitions wherein m is 1.

In a further embodiment, the methods comprise a compound of formula 46 and the attendant definitions wherein R₁ is Cl.

In a further embodiment, the methods comprise a compound of formula 46 and the attendant definitions wherein o is 1.

In a further embodiment, the methods comprise a compound of formula 46 and the attendant definitions wherein R₂ is Cl.

In a further embodiment, the methods comprise a compound of formula 46 and the attendant definitions wherein p is 3.

In a further embodiment, the methods comprise a compound of formula 46 and the attendant definitions wherein R₃ is OH or I.

In a further embodiment, the methods comprise a compound of formula 46 and the attendant definitions wherein n is 0 and m is 1.

In a further embodiment, the methods comprise a compound of formula 46 and the attendant definitions wherein n is 0, m is 1, and o is 1.

In a further embodiment, the methods comprise a compound of formula 46 and the attendant definitions wherein n is 0, m is 1, o is 1, and R₁ is Cl.

In a further embodiment, the methods comprise a compound of formula 46 and the attendant definitions wherein n is 0, m is 1, o is 1, R₁ is Cl, and p is 3.

In a further embodiment, the methods comprise a compound of formula 46 and the attendant definitions wherein n is 0, m is 1, o is 1, R₁ is Cl, p is 3, and R₂ is OH or I.

In another embodiment, a xenohormetic compound may also be a compound represented by formula 47 having xenohormetic activity:

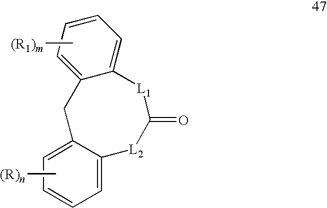

wherein, independently for each occurrence:

R and R₁ are hydroxy, amino, cyano, halide, OR₅, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR₄, or S;

R₄ is H or a substituted or unsubstituted alkyl, aryl, aralkyl, heterocyclyl, heterocyclylalkyl, heteroaryl, or heteroaralkyl;

R₅ is alkyl, —SO₃H, monosaccharide, oligosaccharide, glycofuranosyl, glycopyranosyl, glucuronosyl, or glucuronide; and

m and n are integers from 0 to 4 inclusive.

In a further embodiment, the methods comprise a compound of formula 47 and the attendant definitions wherein n is 2.

In a further embodiment, the methods comprise a compound of formula 47 and the attendant definitions wherein R is methyl or t-butyl.

In a further embodiment, the methods comprise a compound of formula 47 and the attendant definitions wherein m is 2.

In a further embodiment, the methods comprise a compound of formula 47 and the attendant definitions wherein R₁ is methyl or t-butyl.

In a further embodiment, the methods comprise a compound of formula 47 and the attendant definitions wherein L₁ is O.

In a further embodiment, the methods comprise a compound of formula 47 and the attendant definitions wherein L₂ is O.

In a further embodiment, the methods comprise a compound of formula 47 and the attendant definitions wherein n is 2 and R is methyl or t-butyl.

In a further embodiment, the methods comprise a compound of formula 47 and the attendant definitions wherein n is 2, R is methyl or t-butyl, and m is 2.

In a further embodiment, the methods comprise a compound of formula 47 and the attendant definitions wherein n is 2, R is methyl or t-butyl, m is 2, and R₁ is methyl or t-butyl.

In a further embodiment, the methods comprise a compound of formula 47 and the attendant definitions wherein n is 2, R is methyl or t-butyl, m is 2, R₁ is methyl or t-butyl, and L₁ is O.

In a further embodiment, the methods comprise a compound of formula 47 and the attendant definitions wherein n is 2, R is methyl or t-butyl, m is 2, R₁ is methyl or t-butyl, L₁ is O, and L₂ is O.

In another embodiment, a xenohormetic compound may also be a compound represented by formula 48 having xenohormetic activity:

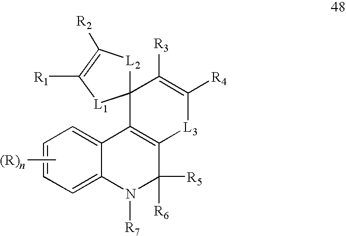

wherein, independently for each occurrence:

R, R₁, R₂, R₃, R₄, R₅, and R₆ are hydroxy, amino, cyano, halide, OR₈, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₇ is H or a substituted or unsubstituted alkyl, acyl, aryl, aralkyl, heterocyclyl, heterocyclylalkyl, heteroaryl, or heteroaralkyl;

Rₛ is alkyl, —SO₃H, monosaccharide, oligosaccharide, glycofuranosyl, glycopyranosyl, glucuronosyl, or glucuronide;

L₁, L₂, and L₃ are O, NR₇, or S and

n is an integer from 0 to 4 inclusive.

In a further embodiment, the methods comprise a compound of formula 48 and the attendant definitions wherein n is 1.

In a further embodiment, the methods comprise a compound of formula 48 and the attendant definitions wherein R is methyl.

In a further embodiment, the methods comprise a compound of formula 48 and the attendant definitions wherein R₁ is C(O)OCH₃.

In a further embodiment, the methods comprise a compound of formula 48 and the attendant definitions wherein R₂ is C(O)OCH₃.

In a further embodiment, the methods comprise a compound of formula 48 and the attendant definitions wherein R₃ is C(O)OCH₃.

In a further embodiment, the methods comprise a compound of formula 48 and the attendant definitions wherein R₄ is C(O)OCH₃.

In a further embodiment, the methods comprise a compound of formula 48 and the attendant definitions wherein R₅ is methyl.

In a further embodiment, the methods comprise a compound of formula 48 and the attendant definitions wherein R₆ is methyl.

In a further embodiment, the methods comprise a compound of formula 48 and the attendant definitions wherein R₇ is C(O)CF₃.

In a further embodiment, the methods comprise a compound of formula 48 and the attendant definitions wherein L₁ is S.

In a further embodiment, the methods comprise a compound of formula 48 and the attendant definitions wherein L₂ is S.

In a further embodiment, the methods comprise a compound of formula 48 and the attendant definitions wherein L₃ is S.

In a further embodiment, the methods comprise a compound of formula 48 and the attendant definitions wherein n is 1 and R is methyl.

In a further embodiment, the methods comprise a compound of formula 48 and the attendant definitions wherein n is 1, R is methyl, and R₁ is C(O)OCH₃.

In a further embodiment, the methods comprise a compound of formula 48 and the attendant definitions wherein n is 1, R is methyl, R₁ is C(O)OCH₃, and R₂ is C(O)OCH₃.

In a further embodiment, the methods comprise a compound of formula 48 and the attendant definitions wherein n is 1, R is methyl, R₁ is C(O)OCH₃, R₂ is C(O)OCH₃, and R₃ is C(O)OCH₃.

In a further embodiment, the methods comprise a compound of formula 48 and the attendant definitions wherein n is 1, R is methyl, R₁ is C(O)OCH₃, R₂ is C(O)OCH₃, R₃ is C(O)OCH₃, and R₄ is C(O)OCH₃.

In a further embodiment, the methods comprise a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, and R₅ is methyl.

In a further embodiment, the methods comprise a compound of formula 48 and the attendant definitions wherein n is 1, R₁ is methyl, R₁ is C(O)OCH₃, R₂ is C(O)OCH₃, R₃ is C(O)OCH₃, R₄ is C(O)OCH₃, R₅ is methyl, and R₆ is methyl.

In a further embodiment, the methods comprise a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, and R₇ is C(O)CF₃.

In a further embodiment, the methods comprise a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, and L₁ is S.

In a further embodiment, the methods comprise a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, L₁ is S, and L₂ is S.

In a further embodiment, the methods comprise a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, L₁ is S, L₂ is S, and L₃ is S.

In another embodiment, a xenohormetic compound may also be a compound represented by formula 49 having xenohormetic activity:

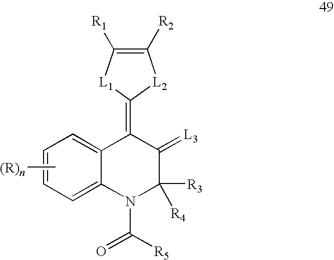

wherein, independently for each occurrence:

R, R₁, R₂, R₃, R₄, and R₅ are hydroxy, amino, cyano, halide, OR₇, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR₆, or S;

R₆ is H or a substituted or unsubstituted alkyl, aryl, aralkyl, heterocyclyl, heterocyclylalkyl, heteroaryl, or heteroaralkyl;

R₇ is alkyl, —SO₃H, monosaccharide, oligosaccharide, glycopyranosyl, glycopyranosyl, glucuronosyl, or glucuronide; and

n is an integer from 0 to 4 inclusive.

In a further embodiment, the methods comprise a compound of formula 49 and the attendant definitions wherein n is 1.

In a further embodiment, the methods comprise a compound of formula 49 and the attendant definitions wherein R is methyl.

In a further embodiment, the methods comprise a compound of formula 49 and the attendant definitions wherein R₁ is C(O)OCH₃.

In a further embodiment, the methods comprise a compound of formula 49 and the attendant definitions wherein R₂ is C(O)OCH₃.

In a further embodiment, the methods comprise a compound of formula 49 and the attendant definitions wherein R₃ is methyl.

In a further embodiment, the methods comprise a compound of formula 49 and the attendant definitions wherein R₄ is methyl.

In a further embodiment, the methods comprise a compound of formula 49 and the attendant definitions wherein R₅ is CH₂CH(CH₃)₂.

In a further embodiment, the methods comprise a compound of formula 49 and the attendant definitions wherein L₁ is S.

In a further embodiment, the methods comprise a compound of formula 49 and the attendant definitions wherein L₂ is S.

In a further embodiment, the methods comprise a compound of formula 49 and the attendant definitions wherein L₃ is S.

In a further embodiment, the methods comprise a compound of formula 49 and the attendant definitions wherein n is 1 and R is methyl.

In a further embodiment, the methods comprise a compound of formula 49 and the attendant definitions wherein n is 1, R is methyl, and R₁ is C(O)OCH₃.

In a further embodiment, the methods comprise a compound of formula 49 and the attendant definitions wherein n is 1, R is methyl, R₁ is C(O)OCH₃, and R₂ is C(O)OCH₃.

In a further embodiment, the methods comprise a compound of formula 49 and the attendant definitions wherein n is 1, R is methyl, R₁ is C(O)OCH₃, R₂ is C(O)OCH₃, and R₃ is methyl.

In a further embodiment, the methods comprise a compound of formula 49 and the attendant definitions wherein n is 1, R is methyl, R₁ is C(O)OCH₃, R₂ is C(O)OCH₃, R₃ is methyl, and R₄ is methyl.

In a further embodiment, the methods comprise a compound of formula 49 and the attendant definitions wherein n is 1, R₁ is methyl, R₁ is C(O)OCH₃, R₂ is C(O)OCH₃, R₃ is methyl, R₄ is methyl, and R₅ is CH₂CH(CH₃)₂.

In a further embodiment, the methods comprise a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, and L₁ is S.

In a further embodiment, the methods comprise a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, and L₁ is S.

In a further embodiment, the methods comprise a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, and L₂ is S.

In a further embodiment, the methods comprise a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, and L₂ is S.

In a further embodiment, the methods comprise a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, L₂ is S, and L₃ is S.

In another embodiment, a xenohormetic compound may also be a compound represented by formula 50 having xenohormetic activity:

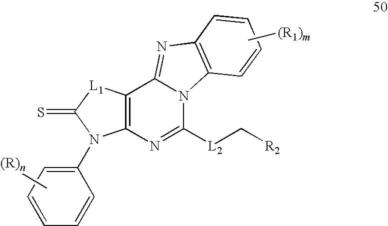

wherein, independently for each occurrence:

R and R₁ are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₄ is alkyl, —SO₃H, monosaccharide, oligosaccharide, glycopyranosyl, glycopyranosyl, glucuronosyl, or glucuronide;

L₁ and L₂ are O, NR₃, or S;

R₃ is H or a substituted or unsubstituted alkyl, aryl, aralkyl, heterocyclyl, heterocyclylalkyl, heteroaryl, or heteroaralkyl;

n is an integer from 0 to 5 inclusive; and

m is an integer from 0 to 4 inclusive.

In a further embodiment, the methods comprise a compound of formula 50 and the attendant definitions wherein n is 1.

In a further embodiment, the methods comprise a compound of formula 50 and the attendant definitions wherein R is CO₂Et.

In a further embodiment, the methods comprise a compound of formula 50 and the attendant definitions wherein m is 0.

In a further embodiment, the methods comprise a compound of formula 50 and the attendant definitions wherein R₂ is cyano.

In a further embodiment, the methods comprise a compound of formula 50 and the attendant definitions wherein L₁ is S.

In a further embodiment, the methods comprise a compound of formula 50 and the attendant definitions wherein L₂ is S.

In a further embodiment, the methods comprise a compound of formula 50 and the attendant definitions wherein n is 1 and R is CO₂Et.

In a further embodiment, the methods comprise a compound of formula 50 and the attendant definitions wherein n is 1, R is CO₂Et, and m is 0.

In a further embodiment, the methods comprise a compound of formula 50 and the attendant definitions wherein n is 1, R is CO₂Et, m is 0, and R₂ is cyano.

In a further embodiment, the methods comprise a compound of formula 50 and the attendant definitions wherein n is 1, R is CO₂Et, m is 0, R₂ is cyano, and L₁ is S.

In a further embodiment, the methods comprise a compound of formula 50 and the attendant definitions wherein n is 1, R is CO₂Et, m is 0, R₂ is cyano, L₁ is S, and L₂ is S.

In another embodiment, a xenohormetic compound may also be a compound represented by formula 51 having xenohormetic activity:

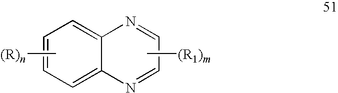

wherein, independently for each occurrence.

R and R₁ are hydroxy, amino, cyano, halide, OR₂, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is alkyl, —SO₃H, monosaccharide, oligosaccharide, glycofuranosyl, glycopyranosyl, glucuronosyl, or glucuronide;

n is an integer from 0 to 4 inclusive; and

m is an integer from 0 to 2 inclusive.

In a further embodiment, the methods comprise a compound of formula 51 and the attendant definitions wherein n is 2.

In a further embodiment, the methods comprise a compound of formula 51 and the attendant definitions wherein R is Cl or trifluoromethyl.

In a further embodiment, the methods comprise a compound of formula 51 and the attendant definitions wherein m is 2.

In a further embodiment, the methods comprise a compound of formula 51 and the attendant definitions wherein R₁ is phenyl.

In a further embodiment, the methods comprise a compound of formula 51 and the attendant definitions wherein n is 2 and R is Cl or trifluoromethyl.

In a further embodiment, the methods comprise a compound of formula 51 and the attendant definitions wherein n is 2, R is Cl or trifluoromethyl, and m is 2.

In a further embodiment, the methods comprise a compound of formula 51 and the attendant definitions wherein n is 2, R is Cl or trifluoromethyl, m is 2, and R₁ is phenyl.

In a further embodiment, the methods comprise a compound of formula 51 and the attendant definitions wherein n is 1.

In a further embodiment, the methods comprise a compound of formula 51 and the attendant definitions wherein R is F.

In a further embodiment, the methods comprise a compound of formula 51 and the attendant definitions wherein R₁ is 4-methylphenyl.

In a further embodiment, the methods comprise a compound of formula 51 and the attendant definitions wherein n is 1 and R is F.

In a further embodiment, the methods comprise a compound of formula 51 and the attendant definitions wherein n is 1, R is F, and m is 2.

In a further embodiment, the methods comprise a compound of formula 51 and the attendant definitions wherein n is 1, R is F, m is 2, and R₁ is 4-methylphenyl.

In another embodiment, a xenohormetic compound may also be a compound represented by formula 52 having xenohormetic activity:

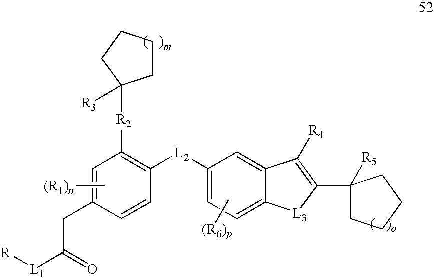

wherein, independently for each occurrence:

R is H or a substituted or unsubstituted alkyl, aryl, aralkyl, heterocyclyl, heterocyclylalkyl, heteroaryl, or heteroaralkyl;

R₁ and R₆ are hydroxy, amino, cyano, halide, OR₇, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is alkylene, alkenylene, or alkynylene;

R₃, R₄, and R₅ are H, hydroxy, amino, cyano, halide, OR₇, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₇ is alkyl, —SO₃H, monosaccharide, oligosaccharide, glycofuranosyl, glycopyranosyl, glucuronosyl, or glucuronide;

L₁, L₂, and L₃ are O, NR, or S;

n and p are integers from 0 to 3 inclusive; and

m and o are integers from 0 to 2 inclusive.

In a further embodiment, the methods comprise a compound of formula 52 and the attendant definitions wherein R is CH₂CH₂OH.

In a further embodiment, the methods comprise a compound of formula 52 and the attendant definitions wherein n is 1.

In a further embodiment, the methods comprise a compound of formula 52 and the attendant definitions wherein R₁ is I.

In a further embodiment, the methods comprise a compound of formula 52 and the attendant definitions wherein R₂ is alkynylene.

In a further embodiment, the methods comprise a compound of formula 52 and the attendant definitions wherein m is 1.

In a further embodiment, the methods comprise a compound of formula 52 and the attendant definitions wherein R₃ is OH.

In a further embodiment, the methods comprise a compound of formula 52 and the attendant definitions wherein R₄ is C(O)OEt.

In a further embodiment, the methods comprise a compound of formula 52 and the attendant definitions wherein o is 1.

In a further embodiment, the methods comprise a compound of formula 52 and the attendant definitions wherein R₅ is OH.

In a further embodiment, the methods comprise a compound of formula 52 and the attendant definitions wherein p is 0.

In a further embodiment, the methods comprise a compound of formula 52 and the attendant definitions wherein L₁ is NH.

In a further embodiment, the methods comprise a compound of formula 52 and the attendant definitions wherein L₂ is O.

In a further embodiment, the methods comprise a compound of formula 52 and the attendant definitions wherein L₃ is O.

In a further embodiment, the methods comprise a compound of formula 52 and the attendant definitions wherein R is CH₂CH₂OH and n is 1.

In a further embodiment, the methods comprise a compound of formula 52 and the attendant definitions wherein R is CH₂CH₂OH, n is 1, and R₁ is I.

In a further embodiment, the methods comprise a compound of formula 52 and the attendant definitions wherein R is CH₂CH₂OH, n is 1, R₁ is I, and R₂ is alkynylene.

In a further embodiment, the methods comprise a compound of formula 52 and the attendant definitions wherein R is CH₂CH₂OH, n is 1, R₁ is I, R₂ is alkynylene, and m is 1.

In a further embodiment, the methods comprise a compound of formula 52 and the attendant definitions wherein R is CH₂CH₂OH, n is 1, R₁ is I, R₂ is alkynylene, m is 1, and R₃ is OH.

In a further embodiment, the methods comprise a compound of formula 52 and the attendant definitions wherein R₁ is CH₂CH₂OH, n is 1, R₁ is I, R₂ is alkynylene, m is 1, R₃ is OH, and R₄ is C(O)OEt.

In a further embodiment, the methods comprise a compound of formula 52 and the attendant definitions wherein R is CH₂CH₂OH, n is 1, R₁ is I, R₂ is alkynylene, m is 1, R₃ is OH, R₄ is C(O)OEt, and o is 1.

In a further embodiment, the methods comprise a compound of formula 52 and the attendant definitions wherein R is CH₂CH₂OH, n is 1, R₁ is I, R₂ is alkynylene, m is 1, R₃ is OH, R₄ is C(O)OEt, o is 1, and R₅ is OH.

In a further embodiment, the methods comprise a compound of formula 52 and the attendant definitions wherein R is CH₂CH₂OH, n is 1, R₁ is I, R₂ is alkynylene, m is 1, R₃ is OH, R₄ is C(O)OEt, o is I, R₅ is OH, and p is O.

In a further embodiment, the methods comprise a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, and L₁ is NH.

In a further embodiment, the methods comprise a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, L₁ is NH, and L₂ is O.

In a further embodiment, the methods comprise a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, L₁ is NH, L₂ is O, and L₃ is O.

In another embodiment, a xenohormetic compound may also be a compound represented by formula 53 having xenohormetic activity:

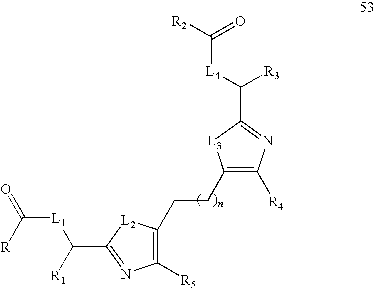

wherein, independently for each occurrence:

R, R₁, R₂, R₃, R₄, and Rₛ are H, hydroxy, amino, cyano, halide, OR₇, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, L₃, and L₄ are O, NR₆, or S;

R₆ is and H, or a substituted or unsubstituted alkyl, aryl, aralkyl, heterocyclyl, heterocyclylalkyl, heteroaryl, or heteroaralkyl;

R₇ is alkyl, —SO₃H, monosaccharide, oligosaccharide, glycofuranosyl, glycopyranosyl, glucuronosyl, or glucuronide; and

n is an integer from 0 to 5 inclusive.

In a further embodiment, the methods comprise a compound of formula 53 and the attendant definitions wherein R is O-t-butyl.

In a further embodiment, the methods comprise a compound of formula 53 and the attendant definitions wherein R₁ is t-butyl.

In a further embodiment, the methods comprise a compound of formula 53 and the attendant definitions wherein R₂ is O-t-butyl.

In a further embodiment, the methods comprise a compound of formula 53 and the attendant definitions wherein R₃ is t-butyl.

In a further embodiment, the methods comprise a compound of formula 53 and the attendant definitions wherein R₄ is C(O)OMe.

In a further embodiment, the methods comprise a compound of formula 53 and the attendant definitions wherein R₅ is C(O)OMe.

In a further embodiment, the methods comprise a compound of formula 53 and the attendant definitions wherein L₁ is NH.

In a further embodiment, the methods comprise a compound of formula 53 and the attendant definitions wherein L₂ is O.

In a further embodiment, the methods comprise a compound of formula 53 and the attendant definitions wherein L₃ is O.

In a further embodiment, the methods comprise a compound of formula 53 and the attendant definitions wherein L₄ is NH.

In a further embodiment, the methods comprise a compound of formula 53 and the attendant definitions wherein n is 1.

In a further embodiment, the methods comprise a compound of formula 53 and the attendant definitions wherein R is O-t-butyl and R₁ is t-butyl.

In a further embodiment, the methods comprise a compound of formula 53 and the attendant definitions wherein R is O-t-butyl, R₁ is t-butyl, and R₂ is O-t-butyl.

In a further embodiment, the methods comprise a compound of formula 53 and the attendant definitions wherein R is O-t-butyl, R₁ is t-butyl, R₂ is O-t-butyl, and R₃ is t-butyl.

In a further embodiment, the methods comprise a compound of formula 53 and the attendant definitions wherein R is O-t-butyl, R₁ is t-butyl, R₂ is O-t-butyl, R₃ is t-butyl, and R₄ is C(O)OMe.

In a further embodiment, the methods comprise a compound of formula 53 and the attendant definitions wherein R is O-t-butyl, R₁ is t-butyl, R₂ is O-t-butyl, R₃ is t-butyl, R₄ is C(O)OMe, and R₅ is C(O)OMe.

In a further embodiment, the methods comprise a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, and L₁ is NH.

In a further embodiment, the methods comprise a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, and L₂ is O.

In a further embodiment, the methods comprise a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, and L₃ is O.

In a further embodiment, the methods comprise a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, L₃ is O, and L₄ is NH.

In a further embodiment, the methods comprise a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, L₃ is O, L₄ is NH, and n is 1.

In another embodiment, a xenohormetic compound may also be a compound represented by formula 54 having xenohormetic activity:

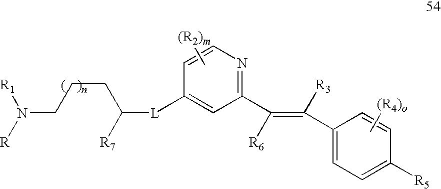

wherein, independently for each occurrence:

R and R₁ are H or a substituted or unsubstituted alkyl, aryl, aralkyl, heterocyclyl, heterocyclylalkyl, heteroaryl, or heteroaralkyl;

R₂, R₄, and R₅ are hydroxy, amino, cyano, halide, OR₈, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₃, R₆, and R₇ are H, hydroxy, amino, cyano, halide, OR₈, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₈ is alkyl, —SO₃H, monosaccharide, oligosaccharide, glycofuranosyl, glycopyranosyl, glucuronosyl, or glucuronide;

L is O, NR, or S;

n and o are integers from 0 to 4 inclusive; and

m is an integer from 0 to 3 inclusive.

In a further embodiment, the methods comprise a compound of formula 54 and the attendant definitions wherein R is ethyl.

In a further embodiment, the methods comprise a compound of formula 54 and the attendant definitions wherein R₁ is ethyl.

In a further embodiment, the methods comprise a compound of formula 54 and the attendant definitions wherein m is 0.

In a further embodiment, the methods comprise a compound of formula 54 and the attendant definitions wherein R₃ is H.

In a further embodiment, the methods comprise a compound of formula 54 and the attendant definitions wherein o is 0.

In a further embodiment, the methods comprise a compound of formula 54 and the attendant definitions wherein R₅ is Cl.

In a further embodiment, the methods comprise a compound of formula 54 and the attendant definitions wherein R₆ is H.

In a further embodiment, the methods comprise a compound of formula 54 and the attendant definitions wherein R₇ is methyl.

In a further embodiment, the methods comprise a compound of formula 54 and the attendant definitions wherein L is NH.

In a further embodiment, the methods comprise a compound of formula 54 and the attendant definitions wherein n is 1.

In a further embodiment, the methods comprise a compound of formula 54 and the attendant definitions wherein R is ethyl and R₁ is ethyl.

In a further embodiment, the methods comprise a compound of formula 54 and the attendant definitions wherein R is ethyl, R₁ is ethyl, and m is 0.

In a further embodiment, the methods comprise a compound of formula 54 and the attendant definitions wherein R is ethyl, R₁ is ethyl, m is 0, and R₃ is H.

In a further embodiment, the methods comprise a compound of formula 54 and the attendant definitions wherein R is ethyl, R₁ is ethyl, m is 0, R₃ is H, and o is 0.

In a further embodiment, the methods comprise a compound of formula 54 and the attendant definitions wherein R is ethyl, R₁ is ethyl, m is 0, R₃ is H, o is 0, and R₅ is Cl.

In a further embodiment, the methods comprise a compound of formula 54 and the attendant definitions wherein R is ethyl, R₁ is ethyl, m is 0, R₃ is H, o is 0, R₅ is Cl, and R₆ is H.

In a further embodiment, the methods comprise a compound of formula 54 and the attendant definitions wherein R is ethyl, R₁ is ethyl, m is 0, R₃ is H, o is 0, R₅ is Cl, R₆ is H, and R₇ is methyl.

In a further embodiment, the methods comprise a compound of formula 54 and the attendant definitions wherein R is ethyl, R₁ is ethyl, m is 0, R₃ is H, o is 0, R₅ is Cl, R₆ is H, R₇ is methyl, and L is NH.

In a further embodiment, the methods comprise a compound of formula 54 and the attendant definitions wherein R is ethyl, R₁ is ethyl, m is 0, R₃ is H, o is 0, R₅ is Cl, R₆ is H, R₇ is methyl, L is NH, and n is 1.

In another embodiment, a xenohormetic compound may also be a compound represented by formula 55 having xenohormetic activity:

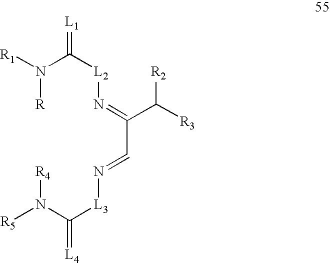

wherein, independently for each occurrence:

R, R₁, R₄, and R₅ are H or a substituted or unsubstituted alkyl, aryl, aralkyl, heterocyclyl, heterocyclylalkyl, heteroaryl, or heteroaralkyl;

R₂ and R₃ are H, hydroxy, amino, cyano, halide, OR₆, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₆ is alkyl, —SO₃H, monosaccharide, oligosaccharide, glycofuranosyl, glycopyranosyl, glucuronosyl, or glucuronide; and

L₁, L₂, L₃, and L₄ are O, NR, or S.

In a further embodiment, the methods comprise a compound of formula 55 and the attendant definitions wherein R is H.

In a further embodiment, the methods comprise a compound of formula 55 and the attendant definitions wherein R₁ is H.

In a further embodiment, the methods comprise a compound of formula 55 and the attendant definitions wherein R₂ is OEt.

In a further embodiment, the methods comprise a compound of formula 55 and the attendant definitions wherein R₃ is methyl.

In a further embodiment, the methods comprise a compound of formula 55 and the attendant definitions wherein R₄ is H.

In a further embodiment, the methods comprise a compound of formula 55 and the attendant definitions wherein R₅ is H.

In a further embodiment, the methods comprise a compound of formula 55 and the attendant definitions wherein L₁ is S.

In a further embodiment, the methods comprise a compound of formula 55 and the attendant definitions wherein L₂ is NH.

In a further embodiment, the methods comprise a compound of formula 55 and the attendant definitions wherein L₃ is NH.

In a further embodiment, the methods comprise a compound of formula 55 and the attendant definitions wherein L₄ is S.

In a further embodiment, the methods comprise a compound of formula 55 and the attendant definitions wherein R is H and R₁ is H.

In a further embodiment, the methods comprise a compound of formula 55 and the attendant definitions wherein R is H, R₁ is H, and R₂ is OEt.

In a further embodiment, the methods comprise a compound of formula 55 and the attendant definitions wherein R is H, R₁ is H, R₂ is OEt, and R₃ is methyl.

In a further embodiment, the methods comprise a compound of formula 55 and the attendant definitions wherein R is H, R₁ is H, R₂ is OEt, R₃ is methyl, and R₄ is H.

In a further embodiment, the methods comprise a compound of formula 55 and the attendant definitions wherein R is H, R₁ is H, R₂ is OEt, R₃ is methyl, R₄ is H, and R₅ is H.

In a further embodiment, the methods comprise a compound of formula 55 and the attendant definitions wherein R is H, R₁ is H, R₂ is OEt, R₃ is methyl, R₄ is H, R₅ is H, and L₁ is S.

In a further embodiment, the methods comprise a compound of formula 55 and the attendant definitions wherein R is H, R₁ is H, R₂ is OEt, R₃ is methyl, R₄ is H, R₅ is H, L₁ is S, and L₂ is NH.

In a further embodiment, the methods comprise a compound of formula 55 and the attendant definitions wherein R is H, R₁ is H, R₂ is OEt, R₃ is methyl, R₄ is H, R₅ is H, L₁ is S, L₂ is NH, and L₃ is NH.

In a further embodiment, the methods comprise a compound of formula 55 and the attendant definitions wherein R is H, R₁ is H, R₂ is OEt, R₃ is methyl, R₄ is H, R₅ is H, L₁ is S, L₂ is NH, L₃ is NH, and L₄ is S.

In another embodiment, a xenohormetic compound may also be a compound represented by formula 56 having xenohormetic activity:

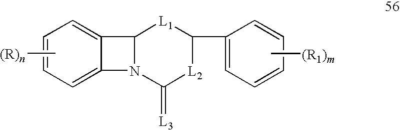

wherein, independently for each occurrence:

R and R₁ are hydroxy, amino, cyano, halide, OR₃, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₃ is alkyl, —SO₃H, monosaccharide, oligosaccharide, glycofuranosyl, glycopyranosyl, glucuronosyl, or glucuronide;

L₁, L₂, and L₃ are O, NR₂, or S;

R₂ is H or a substituted or unsubstituted alkyl, aryl, aralkyl, heterocyclyl, heterocyclylalkyl, heteroaryl, or heteroaralkyl;

n is an integer from 0 to 4 inclusive; and

m is an integer from 0 to 5 inclusive.

In a further embodiment, the methods comprise a compound of formula 56 and the attendant definitions wherein n is 0.

In a further embodiment, the methods comprise a compound of formula 56 and the attendant definitions wherein m is 0.

In a further embodiment, the methods comprise a compound of formula 56 and the attendant definitions wherein L₁ is NH.

In a further embodiment, the methods comprise a compound of formula 56 and the attendant definitions wherein Lₐ is S.

In a further embodiment, the methods comprise a compound of formula 56 and the attendant definitions wherein L₃ is S.

In a further embodiment, the methods comprise a compound of formula 56 and the attendant definitions wherein m is 0 and n is 0.

In a further embodiment, the methods comprise a compound of formula 56 and the attendant definitions wherein m is 0, n is 0, and L₁ is NH.

In a further embodiment, the methods comprise a compound of formula 56 and the attendant definitions wherein m is 0, n is 0, L₁ is NH, and L₂ is S.

In a further embodiment, the methods comprise a compound of formula 56 and the attendant definitions wherein m is 0, n is 0, L₁ is NH, L₂ is S, and L₃ is S.

In another embodiment, a xenohormetic compound may also be a compound represented by formula 57 having xenohormetic activity:

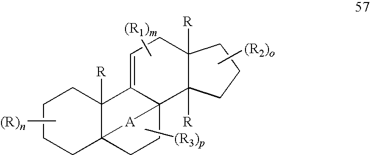

wherein, independently for each occurrence:

R, R₁, R₂, and R₃ are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₃ is alkyl, —SO₃H, monosaccharide, oligosaccharide, glycofuranosyl, glycopyranosyl, glucuronosyl, or glucuronide;

A is alkylene, alkenylene, or alkynylene;

n is an integer from 0 to 8 inclusive;

m is an integer from 0 to 3 inclusive;

o is an integer from 0 to 6 inclusive; and

p is an integer from 0 to 4 inclusive.

In a further embodiment, the methods comprise a compound of formula 57 and the attendant definitions wherein n is 2.

In a further embodiment, the methods comprise a compound of formula 57 and the attendant definitions wherein R is OH or methyl.

In a further embodiment, the methods comprise a compound of formula 57 and the attendant definitions wherein m is 1.

In a further embodiment, the methods comprise a compound of formula 57 and the attendant definitions wherein R₁ is methyl.

In a further embodiment, the methods comprise a compound of formula 57 and the attendant definitions wherein o is 1.

In a further embodiment, the methods comprise a compound of formula 57 and the attendant definitions wherein R₂ is C(O)CH₃.

In a further embodiment, the methods comprise a compound of formula 57 and the attendant definitions wherein p is 2.

In a further embodiment, the methods comprise a compound of formula 57 and the attendant definitions wherein R₃ is CO₂H.

In a further embodiment, the methods comprise a compound of formula 57 and the attendant definitions wherein A is alkenylene.

In a further embodiment, the methods comprise a compound of formula 57 and the attendant definitions wherein n is 2 and R is OH or methyl.

In a further embodiment, the methods comprise a compound of formula 57 and the attendant definitions wherein n is 2, R is OH or methyl, and m is 1.

In a further embodiment, the methods comprise a compound of formula 57 and the attendant definitions wherein n is 2, R is OH or methyl, m is 1, and R₁ is methyl.

In a further embodiment, the methods comprise a compound of formula 57 and the attendant definitions wherein n is 2, R is OH or methyl, m is 1, R₁ is methyl, and o is 1.

In a further embodiment, the methods comprise a compound of formula 57 and the attendant definitions wherein n is 2, R is OH or methyl, m is 1, R₁ is methyl, o is 1, and R₂ is C(O)CH₃.

In a further embodiment, the methods comprise a compound of formula 57 and the attendant definitions wherein n is 2, R is OH or methyl, m is 1, R₁ is methyl, o is 1, R₂ is C(O)CH₃, and p is 2.

In a further embodiment, the methods comprise a compound of formula 57 and the attendant definitions wherein n is 2, R is OH or methyl, m is 1, R₁ is methyl, o is 1, R₂ is C(O)CH₃, p is 2, and R₃ is CO₂H.

In a further embodiment, the methods comprise a compound of formula 57 and the attendant definitions wherein n is 2, R is OH or methyl, m is 1, R₁ is methyl, o is 1, R₂ is C(O)CH₃, p is 2, R₃ is CO₂H, and A is alkenylene.

In another embodiment, a xenohormetic compound may also be a compound represented by formula 58 having xenohormetic activity:

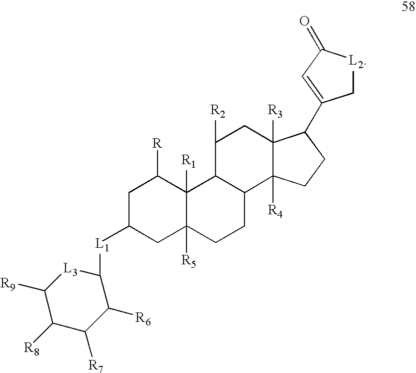

wherein, independently for each occurrence:

R, R₁, R₂, R₃, R₄, R₅, R₆, R₇, R₈, and R₉ are hydroxy, amino, cyano, halide, OR₁₁; ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁₁ is alkyl, —SO₃H, monosaccharide, oligosaccharide, glycofuranosyl, glycopyranosyl, glucuronosyl, or glucuronide;

L₁, L₂ᵢ and L₃ are O, NR₁₀, or S; and

R₁₀ is H or a substituted or unsubstituted alkyl, aryl, aralkyl, heterocyclyl, heterocyclylalkyl, heteroaryl, or heteroaralkyl.

In a further embodiment, the methods comprise a compound of formula 58 and the attendant definitions wherein R is OH.

In a further embodiment, the methods comprise a compound of formula 58 and the attendant definitions wherein R₁ is CH₂OH.

In a further embodiment, the methods comprise a compound of formula 58 and the attendant definitions wherein R₂ is OH.

In a further embodiment, the methods comprise a compound of formula 58 and the attendant definitions wherein R₃ is methyl.

In a further embodiment, the methods comprise a compound of formula 58 and the attendant definitions wherein R₄ is OH.

In a further embodiment, the methods comprise a compound of formula 58 and the attendant definitions wherein R₅ is OH.

In a further embodiment, the methods comprise a compound of formula 58 and the attendant definitions wherein R₆ is OH.

In a further embodiment, the methods comprise a compound of formula 58 and the attendant definitions wherein R₇ is OH.

In a further embodiment, the methods comprise a compound of formula 58 and the attendant definitions wherein R₈ is OH.

In a further embodiment, the methods comprise a compound of formula 58 and the attendant definitions wherein R₉ is methyl.

In a further embodiment, the methods comprise a compound of formula 58 and the attendant definitions wherein L₁ is O.

In a further embodiment, the methods comprise a compound of formula 58 and the attendant definitions wherein L₂ is O.

In a further embodiment, the methods comprise a compound of formula 58 and the attendant definitions wherein L₃ is O.

In a further embodiment, the methods comprise a compound of formula 58 and the attendant definitions wherein R is OH and R₁ is CH₂OH.

In a further embodiment, the methods comprise a compound of formula 58 and the attendant definitions wherein R is OH, R₁ is CH₂OH, and R₂ is OH.

In a further embodiment, the methods comprise a compound of formula 58 and the attendant definitions wherein R is OH, R₁ is CH₂OH, R₂ is OH, and R₃ is methyl.

In a further embodiment, the methods comprise a compound of formula 58 and the attendant definitions wherein R is OH, R₁ is CH₂OH, R₂ is OH, R₃ is methyl, and R₄ is OH.

In a further embodiment, the methods comprise a compound of formula 58 and the attendant definitions wherein R is OH, R₁ is CH₂OH, R₂ is OH, R₃ is methyl, R₄ is OH, and R₅ is OH.

In a further embodiment, the methods comprise a compound of formula 58 and the attendant definitions wherein R is OH, R₁ is CH₂OH, R₂ is OH, R₃ is methyl, R₄ is OH, R₅ is OH, and R₆ is OH.

In a further embodiment, the methods comprise a compound of formula 58 and the attendant definitions wherein R is OH, R₁ is CH₂OH, R₂ is OH, R₃ is methyl, R₄ is OH, R₅ is OH, R₆ is OH, and R₇ is OH.

In a further embodiment, the methods comprise a compound of formula 58 and the attendant definitions wherein R is OH, R₁ is CH₂OH, R₂ is OH, R₃ is methyl, R₄ is OH, R₅ is OH, R₆ is OH, R₇ is OH, and R₈ is OH.

In a further embodiment, the methods comprise a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, and R₉ is methyl.

In a further embodiment, the methods comprise a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, and L₁ is O.

In a further embodiment, the methods comprise a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, L₁ is O, and L₂ is O.

In a further embodiment, the methods comprise a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, L₁ is O, L₂ is O, and L₃ is O.

In another embodiment, a xenohormetic compound may also be a compound represented by formula 59 having xenohormetic activity:

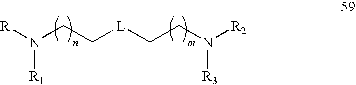

wherein, independently for each occurrence:

R, R₁, R₂, and R₃ are H or a substituted or unsubstituted alkyl, aryl, aralkyl, heterocyclyl, heterocyclylalkyl, heteroaryl, or heteroaralkyl;

L is O, NR, S, or Se; and

n and m are integers from 0 to 5 inclusive.

In a further embodiment, the methods comprise a compound of formula 59 and the attendant definitions wherein R is H.

In a further embodiment, the methods comprise a compound of formula 59 and the attendant definitions wherein R₁ is H.

In a further embodiment, the methods comprise a compound of formula 59 and the attendant definitions wherein R₂ is H.

In a further embodiment, the methods comprise a compound of formula 59 and the attendant definitions wherein R₃ is H.

In a further embodiment, the methods comprise a compound of formula 59 and the attendant definitions wherein L is Se.

In a further embodiment, the methods comprise a compound of formula 59 and the attendant definitions wherein n is 1.

In a further embodiment, the methods comprise a compound of formula 59 and the attendant definitions wherein m is 1.

In a further embodiment, the methods comprise a compound of formula 59 and the attendant definitions wherein R is H and R₁ is H.

In a further embodiment, the methods comprise a compound of formula 59 and the attendant definitions wherein R is H, R₁ is H, and R₂ is H.

In a further embodiment, the methods comprise a compound of formula 59 and the attendant definitions wherein R is H, R₁ is H, R₂ is H, and R₃ is H.

In a further embodiment, the methods comprise a compound of formula 59 and the attendant definitions wherein R is H, R₁ is H, R₂ is H, R₃ is H, and L is Se.

In a further embodiment, the methods comprise a compound of formula 59 and the attendant definitions wherein R is H, R₁ is H, R₂ is H, R₃ is H, L is Se, and n is 1.

In a further embodiment, the methods comprise a compound of formula 59 and the attendant definitions wherein R is H, R₁ is H, R₂ is H, R₃ is H, L is Se, n is 1, and m is 1.

In another embodiment, a xenohormetic compound may also be a compound represented by formula 60 having xenohormetic activity:

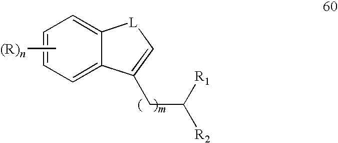

wherein, independently for each occurrence:

R is hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₂ are H, hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₄ is alkyl, —SO₃H, monosaccharide, oligosaccharide, glycofuranosyl, glycopyranosyl, glucuronosyl, or glucuronide;

L is O, NR₃, S, or SO₂;

R₃ is H or a substituted or unsubstituted alkyl, aryl, aralkyl, heterocyclyl, heterocyclylalkyl, heteroaryl, or heteroaralkyl;

n is an integer from 0 to 4 inclusive; and

m is an integer from 1 to 5 inclusive.

In a further embodiment, the methods comprise a compound of formula 60 and the attendant definitions wherein n is 1.

In a further embodiment, the methods comprise a compound of formula 60 and the attendant definitions wherein R is Cl.

In a further embodiment, the methods comprise a compound of formula 60 and the attendant definitions wherein R₁ is NH₂.

In a further embodiment, the methods comprise a compound of formula 60 and the attendant definitions wherein R₂ is CO₂H.

In a further embodiment, the methods comprise a compound of formula 60 and the attendant definitions wherein L is SO₂.

In a further embodiment, the methods comprise a compound of formula 60 and the attendant definitions wherein m is 1.

In a further embodiment, the methods comprise a compound of formula 60 and the attendant definitions wherein n is 1 and R is Cl.

In a further embodiment, the methods comprise a compound of formula 60 and the attendant definitions wherein n is 1, R is Cl, and R₁ is NH₂;

In a further embodiment, the methods comprise a compound of formula 60 and the attendant definitions wherein n is 1, R is Cl, R₁ is NH₂, and R₂ is CO₂H.

In a further embodiment, the methods comprise a compound of formula 60 and the attendant definitions wherein n is 1, R is Cl, R₁ is NH₂, R₂ is CO₂H, and L is SO₂.

In a further embodiment, the methods comprise a compound of formula 60 and the attendant definitions wherein n is 1, R is Cl, R₁ is NH₂, R₂ is CO₂H, L is SO₂, and m is 1.

In another embodiment, a xenohormetic compound may also be a compound represented by formula 61 having xenohormetic activity:

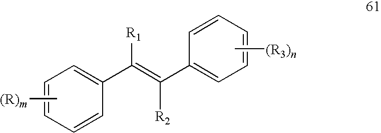

wherein, independently for each occurrence:

R, R₁, R₂, and R₃ are H, hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₄ is alkyl, —SO₃H, monosaccharide; oligosaccharide, glycofuranosyl, glycopyranosyl, glucuronosyl, or glucuronide; and

n and m are integers from 0 to 5 inclusive.

In a further embodiment, the methods comprise a compound of formula 61 and the attendant definitions wherein n is 2.

In a further embodiment, the methods comprise a compound of formula 61 and the attendant definitions wherein R is 3-hydroxy and 5-hydroxy.

In a further embodiment, the methods comprise a compound of formula 61 and the attendant definitions wherein R₁ is H.

In a further embodiment, the methods comprise a compound of formula 61 and the attendant definitions wherein R₂ is H.

In a further embodiment, the methods comprise a compound of formula 61 and the attendant definitions wherein m is 0.

In a further embodiment, the methods comprise a compound of formula 61 and the attendant definitions wherein m is 1.

In a further embodiment, the methods comprise a compound of formula 61 and the attendant definitions wherein R₃ is 4-hydroxy.

In a further embodiment, the methods comprise a compound of formula 61 and the attendant definitions wherein R₃ is 4-methoxy.

In a further embodiment, the methods comprise a compound of formula 61 and the attendant definitions wherein n is 2 and R is 3-hydroxy and 5-hydroxy.

In a further embodiment, the methods comprise a compound of formula 61 and the attendant definitions wherein n is 2, R is 3-hydroxy and 5-hydroxy, and R₁ is H.

In a further embodiment, the methods comprise a compound of formula 61 and the attendant definitions wherein n is 2, R is 3-hydroxy and 5-hydroxy, R₁ is H, and R₂ is H.

In a further embodiment, the methods comprise a compound of formula 61 and the attendant definitions wherein n is 2, R is 3-hydroxy and 5-hydroxy, R₁ is H, R₂ is H, and m is 0.

In a further embodiment, the methods comprise a compound of formula 61 and the attendant definitions wherein n is 2, R is 3-hydroxy and 5-hydroxy, R₁ is H, R₂ is H, and m is 1.

In a further embodiment, the methods comprise a compound of formula 61 and the attendant definitions wherein n is 2, R is 3-hydroxy and 5-hydroxy, R₁ is H, R₂ is H, m is 1, and R₃ is 4-hydroxy.

In a further embodiment, the methods comprise a compound of formula 61 and the attendant definitions wherein n is 2, R is 3-hydroxy and 5-hydroxy, R₁ is H, R₂ is H, m is 1, and R₃ is 4-methoxy.

In another embodiment, a xenohormetic compound may also be a compound represented by formula 62 having xenohormetic activity:

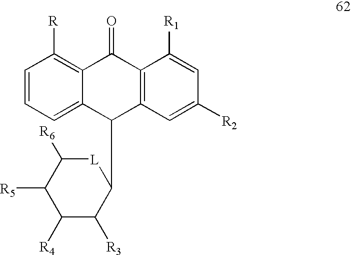

wherein, independently for each occurrence:

R, R₁, R₂, R₃, R₄, R₅, and R₆ are H, hydroxy, amino, cyano, OR₈, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R8 is alkyl, —SO₃H, monosaccharide, oligosaccharide, glycofuranosyl, glycopyranosyl, glucuronosyl, or glucuronide;

L is O, NR₇, or S; and

R₇ is H or a substituted or unsubstituted alkyl, aryl, aralkyl, heterocyclyl, heterocyclylalkyl, heteroaryl, or heteroaralkyl.

In a further embodiment, the methods comprise a compound of formula 62 and the attendant definitions wherein R is OH.

In a further embodiment, the methods comprise a compound of formula 62 and the attendant definitions wherein R₁ is OH.

In a further embodiment, the methods comprise a compound of formula 62 and the attendant definitions wherein R₂ is CH₂OH.

In a further embodiment, the methods comprise a compound of formula 62 and the attendant definitions wherein R₃ is OH.

In a further embodiment, the methods comprise a compound of formula 62 and the attendant definitions wherein R₄ is OH.

In a further embodiment, the methods comprise a compound of formula 62 and the attendant definitions wherein R₅ is OH.

In a further embodiment, the methods comprise a compound of formula 62 and the attendant definitions wherein R₆ is CH₂OH.

In a further embodiment, the methods comprise a compound of formula 62 and the attendant definitions wherein L is O.

In a further embodiment, the methods comprise a compound of formula 62 and the attendant definitions wherein R is OH and R₁ is OH.

In a further embodiment, the methods comprise a compound of formula 62 and the attendant definitions wherein R is OH, R₁ is OH, and R₂ is CH₂OH.

In a further embodiment, the methods comprise a compound of formula 62 and the attendant definitions wherein R is OH, R₁ is OH, R₂ is CH₂OH, and R₃ is OH.

In a further embodiment, the methods comprise a compound of formula 62 and the attendant definitions wherein R is OH, R₁ is OH, R₂ is CH₂OH, R₃ is OH, and R₄ is OH.

In a further embodiment, the methods comprise a compound of formula 62 and the attendant definitions wherein R is OH, R₁ is OH, R₂ is CH₂OH, R₃ is OH, R₄ is OH, and R₅ is OH.

In a further embodiment, the methods comprise a compound of formula 62 and the attendant definitions wherein R is OH, R₁ is OH, R₂ is CH₂OH, R₃ is OH, R₄ is OH, R₅ is OH, and R₆ is CH₂OH.

In a further embodiment, the methods comprise a compound of formula 62 and the attendant definitions wherein R is OH, R₁ is OH, R₂ is CH₂OH, R₃ is OH, R₄ is OH, R₅ is OH, R₄ is CH₂OH, and L is O.

In another embodiment, a xenohormetic compound may also be a compound represented by formula 63 having xenohormetic activity:

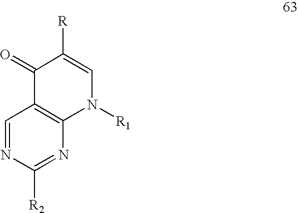

wherein, independently for each occurrence:

R, R₁, and R₂ are H, hydroxy, amino, cyano, halide, OR₃, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and

R₃ is alkyl, —SO₃H, monosaccharide, oligosaccharide, glycopyranosyl, glycopyranosyl, glucuronosyl, or glucuronide.

In a further embodiment, the methods comprise a compound of formula 63 and the attendant definitions wherein R is CO₂H.

In a further embodiment, the methods comprise a compound of formula 63 and the attendant definitions wherein R₁ is ethyl.

In a further embodiment, the methods comprise a compound of formula 63 and the attendant definitions wherein R₂ is N-1-pyrrolidine.

In a further embodiment, the methods comprise a compound of formula 63 and the attendant definitions wherein R is CO₂H and R₁ is ethyl.

In a further embodiment, the methods comprise a compound of formula 63 and the attendant definitions wherein R is CO₂H and R₂ is N-1-pyrrolidine.

In a further embodiment, the methods comprise a compound of formula 63 and the attendant definitions wherein R₁ is ethyl and R₂ is N-1-pyrrolidine.

In a further embodiment, the methods comprise a compound of formula 63 and the attendant definitions wherein R is CO₂H, R₁ is ethyl, and R₂ is N-1-pyrrolidine.

In another embodiment, a xenohormetic compound may also be a compound represented by formula 64 having xenohormetic activity:

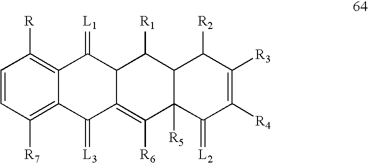

wherein, independently for each occurrence:

R, R₁, R₂, R₃, R₄, R₅, R₆, and R₇ are H, hydroxy, amino, cyano, halide, OR₉, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₉ is alkyl, —SO₃H, monosaccharide, oligosaccharide, glycofuranosyl, glycopyranosyl, glucuronosyl, or glucuronide;

L₁, L₂, and L₃ are CH₂, O, NR₈, or S; and

R₈ is H or a substituted or unsubstituted alkyl, aryl, aralkyl, heterocyclyl, heterocyclylalkyl, heteroaryl, or heteroaralkyl.

In a further embodiment, the methods comprise a compound of formula 64 and the attendant definitions wherein R is Cl.

In a further embodiment, the methods comprise a compound of formula 64 and the attendant definitions wherein R is H.

In a further embodiment, the methods comprise a compound of formula 64 and the attendant definitions wherein R₁ is OH.

In a further embodiment, the methods comprise a compound of formula 64 and the attendant definitions wherein R₂ is N(Me)₂.

In a further embodiment, the methods comprise a compound of formula 64 and the attendant definitions wherein R₃ is OH.

In a further embodiment, the methods comprise a compound of formula 64 and the attendant definitions wherein R₄ is C(O)NH₂.

In a further embodiment, the methods comprise a compound of formula 64 and the attendant definitions wherein R₅ is OH.

In a further embodiment, the methods comprise a compound of formula 64 and the attendant definitions wherein R₆ is OH.

In a further embodiment, the methods comprise a compound of formula 64 and the attendant definitions wherein R₇ is OH.

In a further embodiment, the methods comprise a compound of formula 64 and the attendant definitions wherein L₁ is CH₂.

In a further embodiment, the methods comprise a compound of formula 64 and the attendant definitions wherein L₂ is O.

In a further embodiment, the methods comprise a compound of formula 64 and the attendant definitions wherein L₃ is O.

In a further embodiment, the methods comprise a compound of formula 64 and the attendant definitions wherein R is Cl and R₁ is OH.

In a further embodiment, the methods comprise a compound of formula 64 and the attendant definitions wherein R is Cl, R₁ is OH, and R₂ is N(Me)₂.

In a further embodiment, the methods comprise a compound of formula 64 and the attendant definitions wherein R is Cl, R₁ is OH, R₂ is N(Me)₂, and R₃ is OH.

In a further embodiment, the methods comprise a compound of formula 64 and the attendant definitions wherein R is Cl, R₁ is OH, R₂ is N(Me)₂, R₃ is OH, and R₄ is C(O)NH₂.

In a further embodiment, the methods comprise a compound of formula 64 and the attendant definitions wherein R is Cl, R₁ is OH, R₂ is N(Me)₂, R₃ is OH, R₄ is C(O)NH₂, and R₅ is OH.

In a further embodiment, the methods comprise a compound of formula 64 and the attendant definitions wherein R is Cl, R₁ is OH, R₂ is N(Me)₂, R₃ is OH, R₄ is C(O)NH₂, R₅ is OH, and R₆ is OH.

In a further embodiment, the methods comprise a compound of formula 64 and the attendant definitions wherein R is Cl, R₁ is OH, R₂ is N(Me)₂, R₃ is OH, R₄ is C(O)NH₂, R₅ is OH, R₆ is OH, and R₇ is OH.

In a further embodiment, the methods comprise a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, and L₁ is CH₂.

In a further embodiment, the methods comprise a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, L₁ is CH₂, and L₂ is O.

In a further embodiment, the methods comprise a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, L₁ is CH₂, L₂ is O, and L₃ is O.

In a further embodiment, the methods comprise a compound of formula 64 and the attendant definitions wherein R is H and R₁ is OH.

In a further embodiment, the methods comprise a compound of formula 64 and the attendant definitions wherein R is H, R₁ is OH, and R₂ is N(Me)₂.

In a further embodiment, the methods comprise a compound of formula 64 and the attendant definitions wherein R is H, R₁ is OH, R₂ is N(Me)₂, and R₃ is OH.

In a further embodiment, the methods comprise a compound of formula 64 and the attendant definitions wherein R is H, R₁ is OH, R₂ is N(Me)₂, R₃ is OH, and R₄ is C(O)NH₂.

In a further embodiment, the methods comprise a compound of formula 64 and the attendant definitions wherein R is H, R₁ is OH, R₂ is N(Me)₂, R₃ is OH, R₄ is C(O)NH₂, and R₅ is OH.

In a further embodiment, the methods comprise a compound of formula 64 and the attendant definitions wherein R is H, R₁ is OH, R₂ is N(Me)₂, R₃ is OH, R₄ is C(O)NH₂, R₅ is OH, and R₆ is OH.

In a further embodiment, the methods comprise a compound of formula 64 and the attendant definitions wherein R is H, R₁ is OH, R₂ is N(Me)₂, R₃ is OH, R₄ is C(O)NH₂, R₅ is OH, R₆ is OH, and R₇ is OH.

In a further embodiment, the methods comprise a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, and L₁ is CH₂.

In a further embodiment, the methods comprise a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, L₁ is CH₂, and L₂ is O.

In a further embodiment, the methods comprise a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, L₁ is CH₂, L₂ is O, and L₃ is O.

In another embodiment, a xenohormetic compound may also be a compound represented by formula 65 having xenohormetic activity:

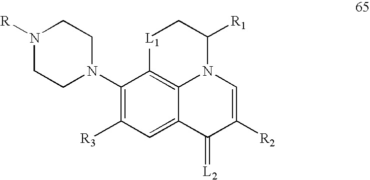

wherein, independently for each occurrence:

R is H or a substituted or unsubstituted alkyl, aryl, aralkyl, heterocyclyl, heterocyclylalkyl, heteroaryl, or heteroaralkyl;

R₁, R₂, and R₃ are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₄ is alkyl, —SO₃H, monosaccharide, oligosaccharide, glycofuranosyl, glycopyranosyl, glucuronosyl, or glucuronide; and

L₁ and L₂ are O, NR, or S.

In a further embodiment, the methods comprise a compound of formula 65 and the attendant definitions wherein R is methyl.

In a further embodiment, the methods comprise a compound of formula 65 and the attendant definitions wherein R₁ is methyl.

In a further embodiment, the methods comprise a compound of formula 65 and the attendant definitions wherein R₂ is CO₂H.

In a further embodiment, the methods comprise a compound of formula 65 and the attendant definitions wherein R₃ is F.

In a further embodiment, the methods comprise a compound of formula 65 and the attendant definitions wherein L₁ is O.

In a further embodiment, the methods comprise a compound of formula 65 and the attendant definitions wherein L₂ is O.

In a further embodiment, the methods comprise a compound of formula 65 and the attendant definitions wherein R is methyl and R₁ is methyl.

In a further embodiment, the methods comprise a compound of formula 65 and the attendant definitions wherein R is methyl, R₁ is methyl, and R₂ is CO₂H.

In a further embodiment, the methods comprise a compound of formula 65 and the attendant definitions wherein R is methyl, R₁ is methyl, R₂ is CO₂H, and R₃ is F.

In a further embodiment, the methods comprise a compound of formula 65 and the attendant definitions wherein R is methyl, R₁ is methyl, R₂ is CO₂H, R₃ is F, and L₁ is O.

In a further embodiment, the methods comprise a compound of formula 65 and the attendant definitions wherein R is methyl, R₁ is methyl, R₂ is CO₂H, R₃ is F, L₁ is O, and L₂ is O.

Exemplary activating compounds are those listed in the appended Tables having a ratio to control rate of more than one. A preferred compound of formula 8 is Dipyridamole; a preferred compound of formula 12 is Hinokitiol; a preferred compound of formula 13 is L-(+)-Ergothioneine; a preferred compound of formula 19 is Caffeic Acid Phenol Ester; a preferred compound of formula 20 is MCI-186 and a preferred compound of formula 21 is HBED (Supplementary Table 6). Activating compounds may also be oxidized forms of the compounds of Table 21.

Also included are pharmaceutically acceptable addition salts and complexes of the compounds of formulas 1-25, 30, and 32-65. In cases wherein the compounds may have one or more chiral centers, unless specified, the compounds contemplated herein may be a single stereoisomer or racemic mixtures of stereoisomers.

In cases in which the compounds have unsaturated carbon-carbon double bonds, both the cis (Z) and trans (E) isomers are contemplated herein. In cases wherein the compounds may exist in tautomeric forms, such as keto-enol tautomers, such as

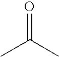

and

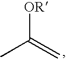

each tautomeric form is contemplated as being included within the methods presented herein, whether existing in equilibrium or locked in one form by appropriate substitution with R′. The meaning of any substituent at any one occurrence is independent of its meaning, or any other substituents meaning, at any other occurrence.

Also included in the methods presented herein are prodrugs of the compounds of formulas 1-25, 30, and 32-65. Prodrugs are considered to be any covalently bonded carriers that release the active parent drug in vivo. Metabolites, such as in vivo degradation products, of the compounds described herein are also included. Resveratrol metabolites include resveratrol-4′-glucoronide, resveratrol-3-O-glucuronide, resveratrol-3-sulphate, and dihydroresveratrol, or any combination thereof.

Analogs and derivatives of the above-described compounds can also be used. For example, derivatives or analogs may make the compounds more stable or improve their ability to traverse cell membranes or being phagocytosed or pinocytosed. Exemplary derivatives include glycosylated derivatives, as described, e.g., in U.S. Pat. No. 6,361,815 for resveratrol. Other derivatives of resveratrol include cis- and trans-resveratrol and conjugates thereof with a saccharide, such as to form a glucoside (see, e.g., U.S. Pat. No. 6,414,037). Glucoside polydatin, referred to as piceid or resveratrol 3-O-beta-D-glucopyranoside, can also be used. Resveratrol polymers, referred to as viniferins, and pterostilbene, a resveratrol analong, can also be used. Saccharides to which compounds may be conjugated include glucose, galactose, maltose, lactose and sucrose. Glycosylated stilbenes are further described in Regev-Shoshani et al. Biochemical J. (published on Apr. 16, 2003 as BJ20030141). Other derivatives of compounds described herein are esters, amides and prodrugs. Esters of resveratrol are described, e.g., in U.S. Pat. No. 6,572,882. Resveratrol and derivatives thereof can be prepared as described in the art, e.g., in U.S. Pat. Nos. 6,414,037; 6,361,815; 6,270,780; 6,572,882; and Brandolini et al. (2002) J. Agric. Food. Chem. 50:7407. Resveratrol may be obtained commercially, e.g., from Sigma.

Similarly to the xenohormetic molecule resveratrol, which activates sirtuins and inhibits several kinases, the xenohormetic molecule quercetin also activates sirtuins and inhibits kinases (see e.g., Davies, S. P., et al. Biochem J. 351:95-105 (2000)). Xenohormetic molecules may also be any phytoalexins and phytoanticipins. Exemplary compounds are set forth in Dixon, R. A., “Natural products and plant disease resistance,” Nature 411:843-847 (2001) and Grayer, R. J. and Harborne, J. B., “A Survey of Antifungal Compounds from Higher Plants, 1982-1993,” Phytochemistry, 37:19-42 (1994). Exemplary phytoalexins may include, but are not limited to, 5-hexylcyclopenta-1,3-dione, 5-octylcyclopenta-1,3-dione, 4,5-methylenedioxy-6-hydroxyaurone, dianthalexins, dianthramides, N-p-hydroxybenzoyl-5-hydroxyanthranilic acid, magnolol, safynol, dehydrosafynol, cichoralexin, mycosinol, scopoletin, ayapin, costunolide, lettucenin A, sesquiterpenes A1 and A2, glyceollins II and III, spirobrassinin, cyclobrassinin, oxymethoxybrassinin, methoxybrassinin, brassinin, dioxybrassinin, brassicanals A-C, cyclobrassinin sulphoxide, brassilexin, camalexin, methoxycamalexin, dihydropinosylvin, demethylbatatasin IV, batatasin W, scopoletin, avenalumin I-III, sakuranetin, momilactones A and B, oryzalexins A-E, oryzalexin S, piceatannol, luteolinidin, apigeninidin 5-caffeylarabinoside, HDIBOA glucoside, medicarpin, isoneorautenol, demethylmedicarpin, desmocarpin, kievitone, diphysolone, ferrerein, nissicarpin, fruticarpin, nissolicarpin, furanodihydrokaempferol, dalbergiodin, phaseollidin, yerenolide, hemigossypol, sanguinarine, benzoic acid, aucuparin, 2′- and 4′-methoxyaucuparin, α- and β-cotonefuran, eriobofuran, α-, β-, and γ-pyrufuran, rhaphiolepsin, 2′,6′-dihydroxyl-4′-methoxyacetophenone, purpurin 1-methyl ether, seselin, scoparone, acteoside, galactosylacteoside, 7-hydroxycalamenene, mansonones psoralen, and bergapten. Other exemplary compounds may include, but are not limited to, sesquiterpene (rishitin, Nicotiana tabacum), diterpene (momilactone A, Oryza sativa), furanoacetylene (wyerone, Vicia faba), flavanone (sakuranetin, Oryza sativa), aurone (Cephalocereus senilis), pterocarpan (maackianin, Cicer arietinum), pterocarpan (medicarpin, Medicago sativa), biphenyl (aucuparin, Malus pumila), benzofuran (Cotoneaster spp.), benzophenanthridine alkaloid (sanguainarine, Papaver bracteatum), benzylisoquinoline alkaloid (berberine, Berberis spp.), indole (camalexin, Arabidopsis thaliana), indole (brassilexin, Brassica spp.), anthranilamide (Dianthus caryophyllus), and elemental sulphur (Theobroma cacao). Xenohormetic molecules may also include derivatives and analogs of these compounds.

Compositions comprising one or more xenohormetic molecules are also provided herein. A composition may comprise at least 2, 3, 5, 10 or more xenohormetic molecules. A composition may be a pharmaceutical composition. A composition may also comprise a xenohormetic molecule and another molecule, such as a vitamin, an anti-oxidant, a carbohydrate, an electrolyte, an amino acid, a mineral, and an energy enhancer.

A xenohormetic molecule may be a “naturally occurring compound,” i.e., a compound that can be found in nature, i.e., a compound that has not been designed by man. A naturally occurring compound may have been made by man or by nature. For example, resveratrol is a naturally-occurring compound. A xenohormetic molecule may also be a “non-naturally occurring compound,” i.e., a compound that is not known to exist in nature or that does not occur in nature. A xenohormetic molecule may be in a “form that is naturally occurring,” i.e., in a form, e.g., a composition, in which it can be found naturally. For example, since resveratrol can be found in red wine, it is present in red wine in a form that is naturally occurring. A compound is not in a form that is naturally occurring if, e.g., the compound has been purified and separated from at least some of the other molecules that are found with the compound in nature.

Additional xenohormetic molecules may be identified by screening molecules to identify those that modulate the stress resistance of cells or organisms or the binding to or activity of one or more xenohormetic target. For example, xenohormetic molecules may be identified by screening molecules to identify those that increase stress resistance of cells or organisms. For example, a cell or organism may be contacted with one or more test molecules and the resistance of the cell or organism to a stress condition is measured in the presence and/or in the absence of one or more test molecules. A higher resistance to the stress condition, e.g., by a factor of at least about 50%, 75%, 100% (2 fold), 3 fold, 5 fold, 10 fold or more, indicates that a test molecule is a xenohormetic molecule.

Another screening assay comprises contacting one or more xenohormetic targets with one or more test molecules and determining the level of interaction of the one or more xenohormetic targets with the one or more test molecules, wherein an interaction between one or more test molecules and targets indicates that one or more test molecules are xenohormetic molecules. An interaction may have a binding affinity of at most about 10⁻⁶M, 10⁻⁷M, 10⁻⁸M, 10⁻⁹M, 10⁻¹⁰M, 10⁻¹¹M, or 10⁻¹²M.

Yet another screening assay comprises contacting one or more xenohormetic targets with one or more test molecules and determining the activity of one or more xenohormetic targets. A screening assay may also comprise contacting a cell or organism comprising one or more xenohormetic targets with one or more test molecules and determining the activity of one or more xenohormetic targets. The xenohormetic targets may be first isolated from the cell or organism or not. A different, e.g., lower or higher, activity of a xenohormetic target relative to that of a control target, e.g., a xenohormetic target from a cell or organism that was not contacted with one or more xenohormetic molecules, indicates that the test molecule is a xenohormetic molecule. For example, if a xenohormetic target is a kinase, e.g., JAK2, pin 1, pim 2, S6K, NLK or Rsk2, a lower activity of the xenohormetic target relative to a control value indicates that the test molecule is a xenohormetic molecule.

Several screening assays, such as those described above, may also be combined. One such combination screening assay comprises screening a test agent, e.g., a molecule, for its ability to protect a cell or organism against a stress condition and further determining the binding or activity of one or more xenohormetic targets in response to the test agent.

Since resveratrol is known to increase insulin sensitivity, one can also use insulin sensitivity as a characteristic to determine whether a molecule is a xenohormetic molecule. Thus, a determination of whether a particular molecule is a xenohormetic molecule may comprise determining whether it modulates the expression or activity of a xenohormetic target and/or whether it increases the sensitivity of a cell to insulin. Other biologic activities of resveratrol may also be used in assays for identifying a xenohormetic molecule. E.g., one may use inhibition of cyclooxygenase (COX2) and/or ornithine decarboxylase (ODC) activity, an increase in plasma antioxidant capacity and/or a decrease in lipid peroxidation, and/or an increased expression of endothelial and/or inducible nitric oxide synthase as characteristic to determine whether a molecule is a xenohormetic molecule.

Xenohormetic molecules may also be isolated or purified from a library of agents or molecules, or from a natural source. For example, a xenohormetic molecule can be purified and isolated from plant material, e.g., leaves, stem, sap, fruit, root, vegetable, flower, flower bud or extract of some or all of these. The plant or plant material is preferably subjected to a stress condition before the purification process. In this regard, the plant or plant material may be subjected to conditions of cold, heat, pressure, radiation, injury or other non-physiological conditions. For example, resveratrol and quercetin, two xenohormetic compounds, are produced in response to fungal infection, injury or high light stress.

Isolating a xenohormetic molecule may comprise subjecting a composition to a screening assay, such as an assay comprising contacting a cell or organism with a composition and determining whether the cell or organism has increased ability to resist a stress condition or determining the activity of one or more xenohormetic targets in the cell or organism. If the screening assay indicates that the composition comprises a xenohormetic molecule, the composition can be separated in two or more fractions, and each fractions tested again. These steps may be repeated a number of times sufficient, e.g., at least about 3 times, 5 times, 10 times, or 30 times, to obtain an essentially pure composition of one or more xenohormetic molecules.

A xenohormetic target refers to a protein whose activity is modulated by a xenohormetic molecule, e.g., resveratrol. Exemplary xenohormetic targets include protein kinases, such as JAK2, Pim-1, Pim-2, S6K, NLK, Rsk2, sirtuins and others described herein. Xenohormetic targets may also be proteins that are not kinases or not a sirtuin. Additional xenohormetic targets may be identified, e.g., by screening kinases that are modulated, e.g., activated or inhibited, by xenohormetic molecules. An assay for identifying a xenohormetic target may comprise contacting a xenohormetic molecule with one or more test proteins, e.g., enzymes, such as kinases, and determining whether the xenohormetic molecule modulates the activity of the test protein. If the xenohormetic molecule modulates the activity of a test protein by at least about 50%, 75%, 2 fold, 3 fold, 5 fold, 10 fold or more, the test protein is likely to be a xenohormetic target.

A set of at least 2, 3, 5, 10 or more xenohormetic targets may constitute a target profile. Determining the status of a target profile in a cell or organism refers to determining the level of activity of at least 2, 3, 5, 10 or more xenohormetic targets in the cell or organism.

Xenohormetic molecules can have multiple independent effects (e.g., reservatrol and quercetin both activate sitruins and inhibit kinases) and can even accomplish the same beneficial effects through multiple mechanisms. For example, a xenohormetic molecule, e.g., resveratrol can increase the expression of an enzyme as well as its activity or alternatively, downregulated the expression of an enzyme and inhibit its activity. This is the case, e.g., of CYP1A1, which is downregulated and inhibited by resveratrol, as well as a Cox-2 and NF-kB (see e.g., J. Baur and D. Sinclair, Nature Reviews 5:493-506 (2006)). Thus, organisms may have evolved to respond in a coordinated way to stress molecules.

In one embodiment, quercetin, an exemplary xenohormetic compound behaves similarly to resveratrol in its ability to inhibit kinases including S6K, PKC, and AMPK (AMP kinase) and inhibition of signaling through a Jak pathway (Davies, S. P. et al., Biochem J. 351:95-105 (2000); Muthian, G. and Bright, J. J., J. Clin. Immunol. 24:542-52 (2004).

In another embodiment, quercetin behaves similarly to resveratrol in its ability to reduce or inhibit IRS-1 expression (Wang, S. et al., J. Nutr. 133:2367-76 (2003)).

### Exemplary Methods

A xenohormetic molecule may be administered to a subject to stimulate the health of (or provide a health benefit to) the subject, e.g., to increase the resistance of the subject to a stress or a diseased condition. A method may comprise administering to a subject, e.g., a subject in need thereof, an effective amount of a xenohormetic molecule, to thereby provide a health benefit, e.g., an increased resistance to a disease or stress condition, to a subject. The method may further comprise determining the effect of the xenohormetic molecule in the subject, e.g., by measuring the activity of one or more xenohormetic targets. An increase or decrease of activity of a xenohormetic target in the subject after administration of the xenohormetic molecule relative to that before administration or that in a subject who has not received the xenohormetic molecule indicates that the subject has gained a health benefit, e.g., an increased resistance to stress.

A subject may be any animal, such as a mammal, e.g., human, canine, feline, ovine, bovine, equine, sheep, or rodent. The methods described herein may also be used to increase the resistance to stress of other animals, such as house pets including fish and birds.

A subject in need of administration of a xenohormetic molecule may be a subject who needs a boost of his health, e.g., a subject who is or has been sick. It may also be a subject who knows that he will encounter a stress condition, e.g., a person who will be exposed to cold, heat, radiation or other non-physiological condition. A subject in need of therapeutic or prophylactic therapy may be a subject who have recently received or are likely to receive a dose of radiation. The dose of radiation is received as part of a work-related or medical procedure, e.g., working in a nuclear power plant, flying an airplane, an X-ray, CAT scan, or the administration of a radioactive dye for medical imaging; in such an embodiment, a xenohormetic molecule is administered as a prophylactic measure. In another embodiment, the radiation exposure is received unintentionally, e.g., as a result of an industrial accident, terrorist act, or act of war involving radioactive material. In such a case, a xenohormetic molecule is preferably administered as soon as possible after the exposure to inhibit the subsequent development of acute radiation syndrome. A subject may also be a subject who has, is or will be ongoing, physical activity, e.g., strenuous exercise.

The health of a subject may also be increased by administering to the subject a food item that contains a xenohormetic molecule. In one method, a subject may ingest a plant or part thereof, which preferably has been subjected to a stress condition to increase the level of one or more xenohormetic molecules therein. Alternatively, a subject may ingest a food or drink into which one or more xenohormetic molecules have been added. A food item may be, e.g., cereal, an energy (e.g., power) bar, a candy, bread, spread and pet food. A food item may also be one that an athletes takes, e.g., to take before or to recover after an athletic event, such as a power bar, energy gels and such.

Xenohormetic molecules may also be prepared in the form of a food or dietary supplement (e.g., a neutraceutical), alone or together with one or more other dietary supplements, such as vitamins and minerals. The dietary supplement may be in the form of a pill or an edible film or strip. Xenohormetic molecules may also be added to liquids and solutions, such as milk, water, juices, tea, and coffee. They may also be added to sports foods and drinks as an athletic supplement. Other ingredients of supplements may include a carbohydrate, a vitamin, an electrolyte, an amino acid, a mineral, an energy enhancer, sodium chloride, potassium chloride, magnesium sulfate, dextrose, sucrose, ascorbic acid, sodium citrate and citric acid.

Xenohormetic molecules may also be administered topically to a subject. As such, one or more xenohormetic molecules may be formulated as part of creams or oils, which may be, e.g., applied to the skin. They may also be included in any cosmetic, cosmeceutical or neutraceutical preparation. They may also be prepared in the form of a patch, e.g., a patch described in U.S. patent application publication number 20050249793.

Also provided herein are methods for preparing food items with health benefits, e.g., anti-stress properties. For example, a plant or product thereof may be subjected to a stress condition, and then used for preparing a food item. The plant may be subjected to the stress condition prior to harvest. Alternatively, the plant and/or a product thereof may be subjected to the stress condition after it has been harvested. A product of a plant, such as fruit or vegetable or a grain, may also be subjected to the stress condition after it has been separated from the plant.

Methods for preparing food items with health benefits, e.g., anti-stress properties, also include adding one or more xenohormetic molecules to a food item that is not related to plants or products thereof. Xenohormetic molecules could be mixed into bakery items, such as bread.

Other methods provided herein involve determining the level of activity of one or more (e.g., at least about 2, 3, 5, 10 or more or all of those in a target profile) xenohormetic targets in a cell or organism or subject to determine whether the cell, organism or subject is healthy or has been subjected to a stress conditions. It is expected that the level of activity of a xenohormetic target would be different if the cell or organism containing it has been exposed to stress. For example, a lower kinase activity of JAK2, Pim 1 or 2, S6K, NLK or Rsk2 in a subject relative to a control would indicate that the subject has been subjected to a stress condition. A difference of at least about 50%, 75%, 100%, 3 fold, 5 fold, 10 fold or more may be a significant difference indicating exposure to a stress condition. Such methods may be used to determine whether a subject has been exposed to an invisible source of stress, such as radiation. Such a person may then take one or more xenohormetic molecules to improve its health and/or increase its resistance to stress.

Also provided are business methods. A business method may comprise one or more of the following steps, in any order. (i) identifying a xenohormetic molecule or target, as described herein; (ii) licensing the right to further develop and/or manufacture the xenohormetic molecule or target; (iii) manufacturing the xenohormetic molecule or target; (iv) incorporating a xenohormetic molecule into, e.g., food, cosmeticals, or nutraceuticals; (v) identifying further xenohormetic molecules or targets or analogs thereof; (vi) conducting animal toxicity profiles on a xenohormetic molecule or target, or an analog thereof; (vii) manufacturing a pharmaceutical or neutraceutical or cosmetic preparation of a xenohormetic molecule or target having a suitable animal toxicity profile; and (viii) marketing the pharmaceutical preparation, e.g., to a healthcare or a neutraceutical provider.

Further provided are methods for conducting a xenohormetic discovery business. A method may comprise one or more of the following steps, in any order. (i) providing one or more assay systems for identifying a potential xenohormetic molecule or target based on the methods described herein; (ii) conducting therapeutic profiling of xenohormetic molecules or targets identified, or further analogs thereof, for efficacy and toxicity in animals; and (iii) formulating a pharmaceutical or neutraceutical or cosmetic preparation including one or more xenohormetic molecules or targets identified as having an acceptable therapeutic profile.

The present description is further illustrated by the following examples, which should not be construed as limiting in any way. The contents of all cited references (including literature references, issued patents, published patent applications and GenBank

Accession numbers as cited throughout this application) are hereby expressly incorporated by reference. When definitions of terms in documents that are incorporated by reference herein conflict with those used herein, the definitions used herein govern.

### EXAMPLES

### Example 1

### Resveratrol is a Specific Kinase Inhibitor

We have screened resveratrol against a panel of 100 kinases and discovered a striking specificity as compared to known kinase inhibitors. Kinase reactions were performed in the presence of 10 or 100 μM ATP and with concentrations of resveratrol of 0.01 μM, 0.03 μM, 0.1 μM, 0.3 μM, 1 μM, 3 μM, 10 μM, 30 μM and 100 μM.

The results are shown in the attached figures. The results show in particular that resveratrol is a specific inhibitor of JAK2, Pim-1, Pim-2, p70S6K, NLK and Rsk2. Rsk2 was inhibited 68% at 20 μM of resveratrol (See Table 2 which shows 32% activity for Rsk2 at 20 μM of resveratrol). This indicates that the response to phytochemicals may be quite specific, as predicted by the xenohormesis hypothesis. This “xenohormetic profile” could be used to identify other health promoting molecules from stressed plants.

TABLE 2
A “xenohormetic profile” of kinases following
treatment with resveratrol (20 μM) or resveratrol-4-glucuronide
(20 μM). Values shown are percent activity as determined by
comparing each compound to a reference file with no resveratrol
or resveratrol-4-glucuronide.

| | Res @ 20 μM | Res 4′-Gluc @ 20 μM |
|---|---|---|
| Abl(h) | 105 | 97 |
| Abl(T315I)(h) | 100 | 93 |
| ALK(h) | 39 | 87 |
| ALK4(h) | 110 | 104 |
| AMPK(r) | 39 | 43 |
| Aurora-A(h) | 65 | 33 |
| CDK1/cyclinB(h) | 67 | 104 |
| CDK2/cyclinA(h) | 80 | 98 |
| CDK2/cyclinE(h) | 80 | 106 |
| CDK3/cyclinE(h) | 75 | 101 |
| CDK5/p25(h) | 64 | 91 |
| CDK5/p35(h) | 77 | 104 |
| CDK6/cyclinD3(h) | 55 | 95 |
| CDK7/cyclinH/MAT1(h) | 95 | 110 |
| CDK9/cyclin T1(h) | 70 | 105 |
| CHK1(h) | 95 | 117 |
| CHK2(h) | 59 | 95 |
| cKit(h) | 68 | 100 |
| CSK(h) | 94 | 100 |
| cSRC(h) | 61 | 54 |
| DAPK1(h) | 71 | 107 |
| DAPK2(h) | 67 | 92 |
| EGFR(h) | 90 | 106 |
| EphB2(h) | 113 | 109 |
| FGFR1(h) | 58 | 45 |
| Fgr(h) | 78 | 67 |
| Flt1(h) | 52 | 76 |
| Flt3(h) | 41 | 48 |
| Fms(h) | 61 | 94 |
| GSK3α(h) | 79 | 57 |
| GSK3β(h) | 92 | 88 |
| IGF-1R(h) | 75 | 87 |
| IKKα(h) | 40 | 113 |
| IKKβ(h) | 116 | 98 |
| IR(h) | 71 | 80 |
| IRR(h) | 40 | 93 |
| IRAK1(h) | 97 | 100 |
| JAK2(h) | 27 | 95 |
| JNK1α1(h) | 89 | 91 |
| JNK2α2(h) | 110 | 123 |
| JNK3(h) | 98 | 105 |
| LIMK1(h) | 84 | 91 |
| LKB1(h) | 102 | 95 |
| MAPK1(h) | 94 | 106 |
| MAPK2(h) | 101 | 105 |
| MEK1(h) | 110 | 120 |
| Met(h) | 114 | 122 |
| MSK1(h) | 58 | 110 |
| MSK2(h) | 60 | 94 |
| MST1(h) | 75 | 106 |
| MST2(h) | 73 | 120 |
| NEK2(h) | 102 | 103 |
| NEK3(h) | 96 | 105 |
| NEK6(h) | 140 | 107 |
| NEK7(h) | 116 | 112 |
| NLK(h) | 30 | 99 |
| p70S6K(h) | 21 | 54 |
| PAK2(h) | 98 | 105 |
| PAK3(h) | 46 | 75 |
| PAK4(h) | 103 | 107 |
| PAK6(h) | 103 | 128 |
| PDGFRα(h) | 114 | 114 |
| PDGFRβ(h) | 119 | 119 |
| PDK1(h) | 108 | 97 |
| Pim-1(h) | 6 | 31 |
| Pim-2(h) | 14 | 76 |
| PKA(h) | 100 | 105 |
| PKBα(h) | 94 | 96 |
| PKBβ(h) | 105 | 94 |
| PKBγ(h) | 89 | 101 |
| PKCα(h) | 114 | 100 |
| PKCβI(h) | 103 | 119 |
| PKCγ(h) | 80 | 77 |
| PKCδ(h) | 92 | 110 |
| PKCθ(h) | 99 | 103 |
| PKCζ(h) | 89 | 104 |
| PKD2(h) | 88 | 113 |
| PKG1β(h) | 56 | 58 |
| PIk3(h) | 95 | 86 |
| Ros(h) | 102 | 105 |
| Rsk1(h) | 70 | 104 |
| Rsk2(h) | 32 | 100 |
| SAPK2a(h) | 100 | 107 |
| SAPK2a(T106M)(h) | 102 | 109 |
| SAPK2b(h) | 100 | 111 |
| SAPK3(h) | 110 | 106 |
| SAPK4(h) | 92 | 104 |
| SGK(h) | 86 | 48 |
| SGK2(h) | 70 | 91 |
| SIK(h) | 76 | 74 |
| Snk(h) | 81 | 87 |
| TAK1(h) | 105 | 113 |
| TBK1(h) | 62 | 80 |
| Tie2(h) | 94 | 76 |
| TrkA(h) | 31 | 32 |
| TrkB(h) | 98 | 100 |
| ZAP-70(h) | 141 | 125 |

### Example 2

### Resveratrol Inhibits the Expression of IRS-1

The expression level of IRS-1 is increased with a high calorie diet and is significantly reduced by resveratrol (for example, adding approximately 22 mg/kg resveratrol to a high calories diet). In addition, IRS-1 is phosphorylated at serine 307 (referred to PIRS-1). The proportion of phosphorylated IRS-1 (PIRS-1) is reduced by a high calorie diet, but increases with resveratrol treatment.

### Example 3

### Aphids Feeding on Stressed Plants Survive Longer than Those Feeding on Non-Stressed Plants

Aphids were kept on either unstressed Arabidopsis or Arabidopsis plants stressed by high light stress, i.e., a bright light for 2-3 days (HL), and their lifespan was measured. The results are shown in Table 3:

TABLE 3

| | Percent of live Aphids on | |
|---|---|---|
| Percent of live Aphids | plants stressed by HL for | Lifespan |
| on unstressed plants | 72 hours | (in days) |
| 100% (41/41) | 100% (36/36) | 3 |
| 100% (41/41) | 100% (36/36) | 4 |
| 93% (38/41) | 100% (36/36) | 5 |
| 93% (38/41) | 100% (36/36) | 6 |
| 90% (37/41) | 100% (36/36) | 7 |
| 90% (37/41) | 100% (36/36) | 9 |
| 88% (36/41) | 97% (35/36) | 11 |
| 88% (36/41) | 92% (33/36) | 12 |
| 83% (34/41) | 92% (33/36) | 14 |
| NA | 86% (31/36) | 16 |

The average number of offspring per aphid on stressed plants is obtained by dividing by the 42 and subsequently 38 as some of the aphids who later died of starvation and were removed from the study produced some offspring. The aphids on unstressed plants are 2 days younger than the aphids on stressed plants, which is why there is no data yet for the % of aphids alive at 16 days.

### Example 4

### Aphids Feeding on Stressed Plants have More Offspring than Those Feeding on Non-Stressed Plants

Aphids were treated as in Example 2 and the number of offspring was counted at different time points. The results are shown in Table 4.

TABLE 4

| | Aphids on | |
|---|---|---|
| | plants stressed | Aphids on |
| | by HL for 72 hours | unstressed plants |
| Earliest onset of offspring | 7 days | 7 days |
| (in age of parent) | | |
| Total # of offspring in 1^{st} | 110 | 13 |
| week | | |
| Avg # of offspring per | 110/42 = 2.62 | 13/41 = 0.32 |
| aphid in the 1^{st} week | | |
| Total # of offspring in 2^{nd} | 316 | 336 |
| week | | |
| Avg # of offspring per | 316/38 = 8.32 | 336/37 = 9.1 |
| aphid in the 2^{nd} week | | |
| Total # of offspring after 2 | 426 | 349 |
| weeks | | |
| Avg # of offspring per | 10.94 | 9.42 |
| aphid (obtained by the sum | | |
| of the averages in each | | |
| week) | | |

### Example 5

### Dose Dependent Inhibition of the Growth of Hematopoietic Cell Line FL5.12 by Resveratrol

Hematopoietic cell line FL5.12 cells were seed at equal density and grown in the presence of IL-3, a growth factor. Cells were then treated for 24 hours with 0.1, 1, 10 and 100 μM resveratrol. Cells were counted with a Coulter particle counter to determine cell inhibition by resveratrol. FIG. 6 is a graph showing dose dependent inhibition of FL5.12 cell growth by resveratrol.

### REFERENCES

1) McCay, C. M. & Crowell, M. F. Prolonging the lifespan. Scientific Monthly 39, 405-414 (1934).
2) Barger, J. L., Walford, R. L. & Weindruch, R. The retardation of aging by caloric restriction: its significance in the transgenic era. Exp Gerontol 38, 1343-51 (2003).
3) Koubova, J. & Guarente, L. How does calorie restriction work? Genes Dev 17, 313-21 (2003).
4) Lamming, D. W., Wood, J. G. & Sinclair, D. A. Small molecules that regulate lifespan: evidence for xenohormesis Mol Microbiol 53, 1003-9 (2004):
5) Langcake, P. & Pryce, R. J. The production of resveratrol by Vitis vinifera and other members of the Vitaceae as a response to infection or injury. Physiol Plant Pathol 9, 77-86 (1976).
6) M. Adrian,* P. Jeandet, A. C. Douillet-Breuil, L. Tesson, and R. Bessis. Stilbene Content of Mature Vitis vinifera Berries in Response to UV-C Elicitation. J. Agric. Food Chem., 48 (12), 6103-61.05 (2000).
7) Fabienne Larronde, Jean P. Gaudillère, Stéphanie Krisa, Alain Decendit, Gerard Deffieux, and Jean M. Mérillon. Airborne Methyl Jismonate Induces Stilbene Accumulation in Leaves and Berries of Grapevine Plants. Am. J. Enol. Vitic. 54:1:63-66 (2003).
8) Schwekendiek A, Pfeffer G, Kindl H. Pine stilbene synthase cDNA, a tool for probing environmental stress. FEBS Lett. 1992 Apr. 13; 301(1):41-4.

### INCORPORATION BY REFERENCE

The contents of all cited references (including literature references, issued patents, published patent applications and GenBank Accession numbers as cited throughout this application) are hereby expressly incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method for determining the presence of a xenohormetic molecule in a composition or identifying a xenohormetic molecule, comprising
(i) contacting a test composition or a test agent with a cell or organism; and
(ii) determining the level of resistance to stress of the cell or organism, wherein a higher level of resistance to stress of the cell or organism that was contacted with the test composition or the test agent relative to that of a cell or organism that was not contacted with the test composition or the test agent indicates that the test composition contains a xenohormetic molecule or that the test agent is a xenohormetic molecule;
optionally wherein the test composition is a plant extract, optionally wherein the plant extract is an extract from a stressed plant, and/or
optionally wherein the test agent is a small molecule, preferably wherein the test agent is obtained from a plant, preferably wherein the agent is obtained from a stressed plant or portions thereof, preferably wherein a stressed plant or portions thereof is a plant or portions thereof that were exposed to injury, infection, cold, heat, U.V., high intensity light, dehydration, radiation or any non-physiological or suboptimal culture condition.

2. A method for isolating a xenohormetic molecule from a composition, comprising determining the presence of a xenohormetic molecule in a test composition according to the method of claim 1, and further comprising (iii) obtaining a subtraction of the composition, and repeating steps (i) and (ii) of claim 1, wherein a higher level of resistance to stress of the cell or organism that was contacted with the subtraction relative to that of a cell or organism that was not contacted with the subtraction indicates that the subtraction contains a xenohormetic molecule,
optionally comprising repeating steps (i)-(iii) at least about 5 times.

3.-6. (canceled)

7. A method for identifying a xenohormetic molecule, comprising
(i) contacting a xenohormetic target with a test agent; and
(ii) determining the level of interaction between the xenohormetic target and the test agent, wherein a significant interaction between the xenohormetic target and the test agent indicates that the test agent is a xenohormetic molecule; or
determining the level of activity of the xenohormetic target in the presence of the test agent, wherein a different level of activity in the presence of test agent relative to the absence of the test agent indicates that the test agent is a xenohormetic molecule,
optionally wherein the xenohormetic target is JAK2, Pim1, Pim2, S6K, NLK, Rsk2 or a sirtuin, and/or
optionally wherein the test agent is a small molecule, preferably wherein the test agent is obtained from a plant, preferably wherein the agent is obtained from a stressed plant or portions thereof, preferably wherein a stressed plant or portions thereof is a plant or portions thereof that were exposed to injury, infection, cold, heat, U.V., high intensity light, dehydration, radiation or any non-physiological or suboptimal culture condition.

8. (canceled)

9. A method for identifying a xenohormetic molecule, comprising
(i) contacting a cell or organism with a test agent; and
(ii) determining the level of activity of a xenohormetic target in the cell or organism, wherein a different level of activity in the presence of test agent relative to the absence of the test agent indicates that the test agent is a xenohormetic molecule, or
determining the status of the xenohormetic target profile of the cell or organism, wherein a different level of activity of the xenohormetic targets in the xenohormetic target profile of the cell or organism that was contacted with the test agent relative to those in a cell or organism that was not contacted with the test agent indicates that the test agent is a xenohormetic molecule,
optionally wherein the xenohormetic target is JAK2, Pim1, Pim2, S6K, NLK, Rsk2 or a sirtuin, and/or
optionally wherein the xenohormetic target profile comprises at least two proteins selected from the group consisting of JAK2, Pim1, Pim2, S6K, NLK, Rsk2 and a sirtuin, and/or
optionally wherein the test agent is a small molecule, preferably, wherein the test agent is obtained from a plant, preferably wherein the agent is obtained from a stressed plant or portions thereof, preferably wherein a stressed plant or portions thereof is a plant or portions thereof that were exposed to injury, infection, cold, heat, U.V., high intensity light, dehydration, radiation or any non-physiological or suboptimal culture condition.

10.-16. (canceled)

17. A method for identifying a xenohormetic target, comprising
(i) contacting a xenohormetic molecule with a test protein; and
(ii) determining the level of interaction between the xenohormetic molecule and the test protein, wherein a significant interaction between the xenohormetic molecule and the test protein indicates that the test protein is a xenohormetic target, or
determining the level of activity of the test protein in the presence of the xenohormetic molecule, wherein a different activity in the presence of xenohormetic molecule relative to the absence of the xenohormetic molecule indicates that the test protein is a xenohormetic target,
optionally wherein the xenohormetic molecule is a STAC, preferably wherein the xenohormetic molecule is resveratrol.

18.-30. (canceled)

31. A method for increasing the amount of xenohormetic molecules in a food item, cosmeceutical, or dietary supplement derived from a plant or product thereof, comprising
subjecting the plant or product thereof to a stress condition prior to preparing the food item, cosmeceutical, or dietary supplement from it.

32.-35. (canceled)
